(19) United States
(12) Patent Application Publication
(10) Pub. No.: US 2026/0027244 A1
(43) Pub. Date: Jan. 29, 2026

(54) SILICON-BASED FLUORIDE ACCEPTOR GROUPS FOR RADIOPHARMACEUTICALS

(71) Applicant: Technische Universität München, Munich (DE)

(72) Inventors: Hans-Jürgen Wester, Munich (DE); Thomas Günther, Munich (DE); Nadine Holzleitner, Munich (DE); Jan-Philip Kunert, Munich (DE); Roswitha Beck, Munich (DE); Markus Frederik Fahnauer, Munich (DE); Sebastian Alexander Fenzel, Munich (DE); Sandra Michaela Dominelli, Munich (DE); León Stopper, Munich (DE); Nicole Urtz-Urban, Munich (DE); Sebastian Fischer, Munich (DE)

(73) Assignee: Technische Universität München, Munich (DE)

(21) Appl. No.: 18/996,504

(22) PCT Filed: Jul. 28, 2023
(86) PCT No.: PCT/EP2023/071064

§ 371 Date: Jan. 17, 2025

(30) Foreign Application Priority Data
Jul. 29, 2022 (EP) 22187845.7

(51) Int. Cl. A61K 51/08; C07B 59/00

(52) U.S. Cl. CPC A61K 51/088; C07B 59/004

(57) ABSTRACT

The invention relates to novel silicon-based fluoride acceptor groups (SiFA groups) of the following formulae, as well as to compounds suitable for use in radiopharmacy comprising such groups

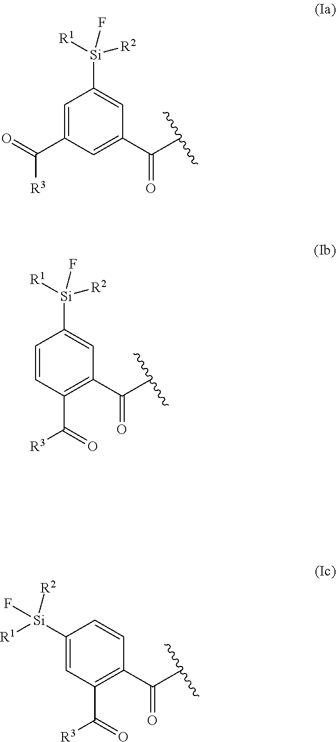

wherein R¹ and R² are each a linear or branched C3 to C10 alkyl group and R³ is selected from (i) —OH or —O⁻, (ii) a sugar moiety or an amino sugar moiety, (iii) an amino acid moiety or an oligopeptide moiety, (iv) a PEG moiety; and from combinations of two or more of (ii), (iii) and (iv).

The invention relates to novel silicon-based fluoride acceptor groups (SiFA groups) which are useful for ¹⁸F-labeling of targeted radiopharmaceuticals via a fast and efficient isotopic exchange reaction.

WO 2019/020831 A1 describes dual mode radiotracers and radiotherapeutics, which combine a silicon-fluoride acceptor group and one or more chelating groups. WO 2022/144463 A1 also relates to compounds which contain a silicon-fluoride acceptor group together with a chelate which comprises a chelated radioactive or non-radioactive metal cation, and which are thus suitable for use as radiotracers or radiotherapeutics. R. Koudih et al., Applied Radiation and Isotopes, 80, 2014, pp. 146-150 disclose an approach for the automated synthesis of N-succinimidyl 3-(di-tert-butyl[¹⁸F]fluorosilyl)benzoate for radiolabeling of peptides and proteins.

In comparison to the previously published SiFA groups, especially the most frequently used p-(di-tert-butyl-(fluorosilyl)benzoic acid, the SiFA groups in accordance with the invention pave the way for a) the development of more hydrophilic, SiFA-based ¹⁸F-labeled ligands with improved in vivo properties, e.g., reduced hepatobiliary excretion, reduced plasma protein binding and hence accelerated blood clearance and lower unspecific binding and b) if required an optimized adaptation to structural requirements of the target molecule (receptor, enzyme etc.) according to structure-activity relationships to enable the development of ligands with improved affinity.

Thus, the invention provides a compound comprising a group selected from a group of formula (Ia), (Ib) and (Ic), or a salt of such a compound:

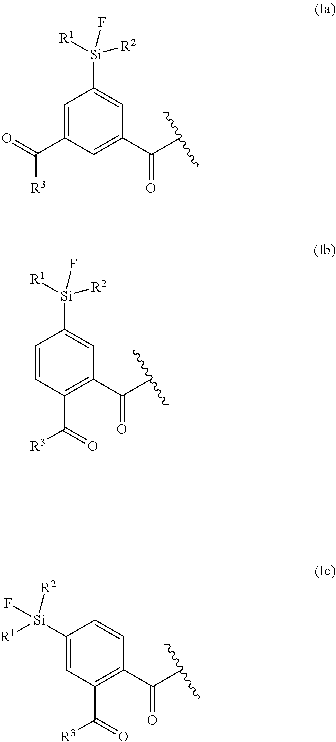

wherein R¹ is a linear or branched C3 to C10 alkyl group, preferably a branched C3 to C10 alkyl group, and more preferably a tert-butyl group; R² is a linear or branched C3 to C10 alkyl group, preferably a branched C3 to C10 alkyl group, and more preferably a tert-butyl group; and R³ is selected from
(i) —OH or —O⁻,
(ii) a sugar moiety or an amino sugar moiety,
(iii) an amino acid moiety or an oligopeptide moiety,
(iv) a PEG moiety;
and from combinations of two or more of (ii), (iii) and (iv);
   and the waved line marks a bond which attaches the group to the remainder of the compound.

It will be understood that, if R³ represents —O⁻, the substituent —C(═O)R³ of the aromatic ring shown in the above formulae is a deprotonated carboxylate group —C(═O)O⁻.

Herein, the groups (Ia) to (Ic) are also referred to as the SiFA groups in accordance with the present invention.

Advantageously, such a SiFA group in accordance with the present invention can be used in a compound comprising a targeting moiety, such as a receptor binding moiety, an enzyme binding substrate or enzyme inhibitor, or a peptide, a protein or an antibody fragment or engineered antigen binding construct, such as a nanobody. The compound comprising the SiFA group and the targeting moiety can be used as a targeted radiopharmaceutical.

Various aspects of the invention and preferred embodiments thereof are summarized in the following items.
1. A compound comprising a group selected from a group of formula (Ia), (Ib) and (Ic), or a salt thereof:

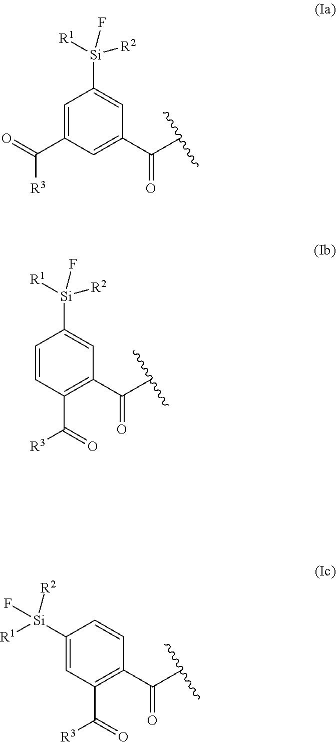

wherein:
R¹ is a linear or branched C3 to C10 alkyl group, preferably a branched C3 to C10 alkyl group, and more preferably a tert-butyl group;
R² is a linear or branched C3 to C10 alkyl group, preferably a branched C3 to C10 alkyl group, and more preferably a tert-butyl group;
R³ is selected from
   (i) —OH or —O⁻,
   (ii) a sugar moiety or an amino sugar moiety,
   (iii) an amino acid moiety or an oligopeptide moiety,
   (iv) a PEG moiety;
   and from combinations of two or more of (ii), (iii) and (iv);
and the waved line marks a bond which attaches the group to the remainder of the compound.
2. The compound in accordance with item 1 which comprises a group of formula (Ia), or a salt of the compound.
3. The compound in accordance with item 1 which is a compound of formula (IIa), (IIb) or (IIc), or a salt of the compound:

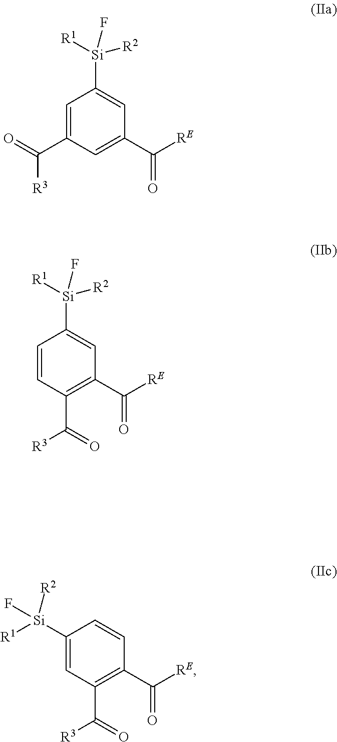

wherein R¹, R², and R³ are defined as in item 1, and R^{E} is a group comprising a targeting moiety.
4. The compound in accordance with item 3 which comprises a group of formula (IIa), or a salt of the compound.
5. The compound or salt in accordance with any of items 1 to 4, wherein R¹ and R² are each a tert-butyl group.
6. The compound or salt in accordance with any of items 1 to 5, wherein the amino acid moiety as R³ is derived from a hydrophilic amino acid that comprises, in addition to an amino group and a carboxyl group, a further basic or acidic functional group, or wherein the oligopeptide moiety as R³ is derived from an oligopeptide with at least one hydrophilic amino acid that comprises, in addition to an amino group and a carboxyl group, a further basic or acidic functional group.
7. The compound or salt in accordance with any of items 1 to 6, wherein the oligopeptide moiety as R³ is a linear or branched moiety comprising 2 to 10, preferably 2 to 5, and more preferably 2 or 3 amino acid moieties.
8. The compound or salt in accordance with any of items 6 or 7, wherein the hydrophilic amino acid is selected from lysine and glutamic acid.
9. The compound or salt in accordance with any of items 1 to 8, wherein the PEG moiety as R³ is a moiety of the formula

—NH—(CH₂—CH₂—O)ₓ—R^{P1},
wherein the nitrogen atom providing an open bond forms an amide bond —NH—C(O)— with the carbon atom to which R³ is attached, X is an integer of 2 to 10, preferably 4 to 10, and is more preferably 8, and R^{P1} is selected from —CH₂—COOH and —CH₂—CH₂—COOH.
10. The compound or salt in accordance with any of items 1 to 9, wherein the sugar moiety or amino sugar moiety as R³ is a residue derived from 6-amino-6-deoxy-D-galactopyranose and corresponding tautomers thereof.
11. The compound or salt in accordance with any of items 1 to 10, wherein R³ is selected from —OH, a lysine moiety, a glutamic acid moiety,
a residue derived from 6-amino-6-deoxy-D-galactopyranose and corresponding tautomers thereof, and
a moiety of the formula

—NH—(CH₂—CH₂—O)ₓ—R^{P1}, and
wherein the nitrogen atom providing an open bond forms an amide bond —NH—C(O)— with the carbon atom to which R³ is attached, X is an integer of 2 to 10, preferably 4 to 10, and is more preferably 8, and R^{P1} is selected from —CH₂—COOH and —CH₂—CH₂—COOH.
12. The compound or salt in accordance with any of items 3 to 11, wherein R^{E} comprises a targeting moiety selected from
a receptor binding moiety, an enzyme binding substrate or enzyme inhibitor, a peptide, a protein or an antibody fragment or engineered antigen binding construct, such as a nanobody.
13. The compound or salt in accordance with item 12, wherein R^{E} comprises a targeting moiety selected from a receptor binding moiety and an enzyme binding substrate or enzyme inhibitor.
14. The compound or salt in accordance with any of items 3 to 13, wherein the targeting moiety is a peptidic moiety.
15. The compound or salt in accordance with any of items 3 to 14, wherein the targeting moiety is a receptor binding moiety which allows the compound of formula (IIa), (IIb) or (IIc) or its salt comprising the targeting moiety to function as a ligand for a receptor selected from a gastrin releasing peptide receptor (GRPR), a C-X-C chemokine receptor type 4 (CXCR4), a somatostatin receptor (SSTR), and a cholecystokinin B receptor (CCK-2R).
16. The compound or salt in accordance with any of items 3 to 14, wherein the targeting moiety is a PSMA binding moiety which allows the compound of formula (IIa), (IIb) or (IIc) or its salt to function as a ligand for prostate-specific membrane antigen (PSMA).
17. The compound or salt in accordance with any of items 3 to 16, wherein the targeting moiety comprised by R^{E} is attached directly to the carbon atom of the carbonyl group in —C(═O)R^{E}.
18. The compound or salt in accordance with any of items 3 to 16, wherein the targeting moiety comprised by R^{E} is attached via a linker group to the carbon atom of the carbonyl group in —C(═O)R^{E}.
19. The compound or salt in accordance with any of items 3 to 16, wherein R^{E} further comprises a chelating moiety or a chelate moiety formed by the chelating moiety and a chelated radioactive or non-radioactive metal cation.
20. The compound or salt in accordance with item 19, wherein the chelating moiety is a moiety which is derived from DOTA or DOTAGA.
21. The compound or salt in accordance with item 19 or 20, wherein the chelated metal cation is a radioactive metal cation.
22. The compound or salt in accordance with any of items 19 to 21, wherein the targeting moiety comprised by R^{E} is attached via a linker group to the carbon atom of the carbonyl group in —C(═O)R^{E} and the chelating moiety or chelate moiety is a part of the linker group.
23. The compound or salt in accordance with any of items 1 to 22, wherein the fluorine attached via a direct covalent bond to the Si atom is [¹⁸F]fluorine.
24. The compound or salt in accordance with any of items 1 to 22, wherein the fluorine attached via a direct covalent bond to the Si atom is [¹⁹F]fluorine.
25. The compound or salt in accordance with any of items 1 to 24 for use as a medicament.
26. A radiopharmaceutical composition comprising a compound or salt in accordance with any of items 1 to 24, optionally in combination with a pharmaceutically acceptable excipient.
27. A compound or salt in accordance with any of items 1 to 24, or a radiopharmaceutical composition in accordance with item 26 for use in a method of diagnosis in vivo of a disease or disorder.
28. The compound, salt or radiopharmaceutical composition for use in accordance with item 27, wherein the method of diagnosis involves nuclear diagnostic imaging.
29. The compound, salt or radiopharmaceutical composition for use in accordance with item 27 or 28, wherein the method of diagnosis involves positron emission tomography (PET).
30. The compound, salt or radiopharmaceutical composition for use in accordance with item 27 or 28, wherein the method of diagnosis involves SPECT.
31. A compound or salt in accordance with any of items 1 to 24, or a radiopharmaceutical composition in accordance with item 26 for use in a method for the treatment of a disease or disorder via radioligand therapy.
32. A method for the preparation of a radiolabeled compound, comprising a step of reacting a compound or salt in accordance with any of items 1 to 22, wherein the fluorine attached via a direct covalent bond to the Si atom is [¹⁹F]fluorine, with [¹⁸F]fluoride to exchange the [¹⁹F]fluorine by [¹⁸F]fluorine.
33. Use of a group of the formula (Ia), (Ib) or (Ic) as defined in any of items 1 or 5 to 11 as a silicon-based fluoride acceptor group for the isotopic exchange of [¹⁹F]fluorine by [¹⁸F]fluorine.
34. Use of a group of the formula (Ia), (Ib) or (Ic) as defined in any of items 1 or 5 to 11 as a silicon-based fluoride acceptor group for the [¹⁸F]labeling of a targeted radiopharmaceutical.

As explained above, the compounds of the invention encompass compounds comprising a group selected from a group of formula (Ia), (Ib) and (Ic), as well as compounds of formula (IIa), (IIb) or (IIc). Moreover, salts, typically pharmaceutically acceptable salts, of the compounds are encompassed by the present invention. Thus, unless indicated to the contrary, any reference to a compound of the invention herein encompasses the compounds comprising a group selected from a group of formula (Ia), (Ib) and (Ic) (and the preferred embodiments of these formulae disclosed herein), and the salts thereof, and compounds of formula (IIa), (IIb) or (IIc) (and the preferred embodiments of these formulae disclosed herein), and the salts thereof. Likewise, any racemates, enantiomers, or diastereomers of any chiral compounds of the invention are encompassed, unless a specific stereochemistry of the compound under consideration is indicated in a specific context.

As one general aspect, the invention provides a compound comprising a group selected from a group of formula (Ia), (Ib) and (Ic), or a salt of such a compound:

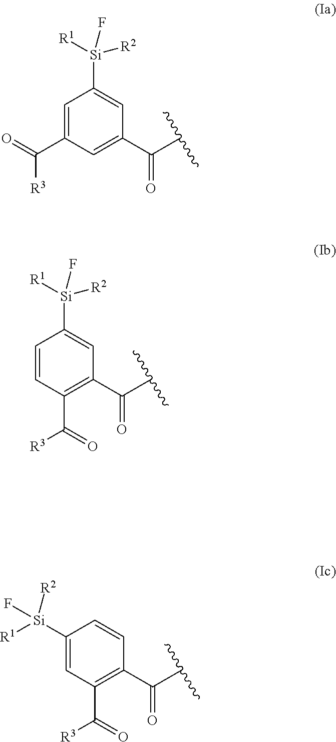

wherein R¹ is a linear or branched C3 to C10 alkyl group, preferably a branched C3 to C10 alkyl group, and more preferably a tert-butyl group; R² is a linear or branched C3 to C10 alkyl group, preferably a branched C3 to C10 alkyl group, and more preferably a tert-butyl group; and R³ is selected from
(i) —OH or —O⁻,
(ii) an amino acid moiety or an oligopeptide moiety,
(iii) a sugar moiety or an amino sugar moiety,
(iv) a PEG moiety;
and from combinations of two or more of (ii), (iii) and (iv);
   and the waved line marks a bond which attaches the group to the remainder of the compound.

As a further general aspect, the invention provides a compound of formula (IIa), (IIb) or (IIc), or a salt of the compound:

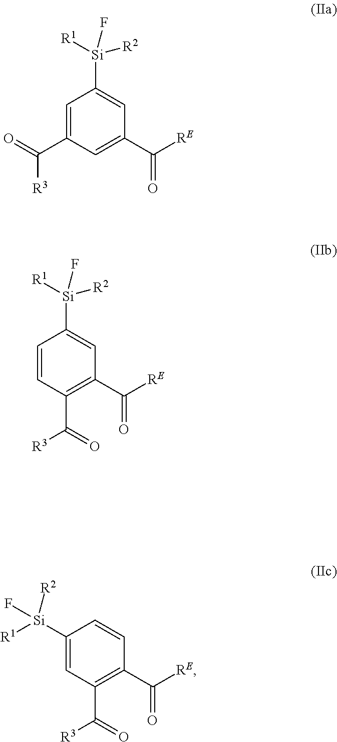

wherein R¹, R², and R³ are as defined for the groups of formula (Ia), (Ib) and (Ic), including any preferred embodiments thereof, and R^{E} is a group comprising a targeting moiety. As will be understood by the skilled reader, the targeting moiety comprised by R^{E} may be attached directly (i.e. by a direct covalent bond) to the carbon atom of the carbonyl group forming the bond with R^{E} in the above formulae, or may optionally be attached via a linker group to this carbon atom. The group R^{E} comprising a targeting moiety allows the compound of formula (IIa), (IIb) or (IIc) or the salt thereof to bind to a target structure, e.g. a target structure overexpressed in a human disease, such as cancer. Thus, compounds or salts in accordance with the invention comprising the group R^{E} are suitable for use as a targeted radiopharmaceutical, in particular as a targeted radiopharmaceutical for in vivo applications in humans.

Among the groups of formula (Ia), (Ib) and (Ic), preference is given to the groups of formula (Ia).

Thus, in accordance with the above, further preference is given to a compound as a compound of the invention which comprises a group of formula (Ia), and wherein R¹ and R² are each a tert-butyl group, or to a salt of such a compound.

Likewise, among the compounds of formula (IIa), (IIb) and (IIc) and their salts, preference is given to the compounds of formula (IIa) and their salts.

Thus, in accordance with the above, further preference is given to a compound (IIa) as a compound of the invention wherein R¹ and R² are each a tert-butyl group, or to a salt of such a compound.

R³ in the above formulae (Ia), (Ib) and (Ic) as well as (IIa), (IIb) and (IIc) is selected from (i) —OH or —O⁻, (ii) an amino acid moiety or an oligopeptide moiety, (iii) a sugar moiety or an amino sugar moiety, (iv) a PEG moiety, and from combinations of two or more of (ii), (iii) and (iv).

As will be understood, if R³ is —OH or —O⁻, the substituent —C(═O)R³ carrying R³ is a carboxyl group —C(═O)OH or a deprotonated carboxylate group —C(═O)O. As exemplary counterions for the deprotonated carboxylate group, reference may be made to the cations listed below which may be present as counterions in salt forms of the compounds in accordance with the invention, e.g. to an ammonium ion or an alkali metal cation. Thus, the compounds of the invention comprise carboxylic acids or carboxylates as salts thereof, e.g. in the form of ammonium or alkali metal salts of the carboxylic acid.

Moreover, R³ can be an amino acid moiety or an oligopeptide moiety, among which preference is given to the amino acid moiety.

As will be understood by the skilled person, an amino acid moiety is a group which can be derived from an amino acid, i.e. from a compound comprising an amino group —NH₂ and a carboxyl group —COOH in the same molecule. Unless indicated otherwise in a specific context, one or more further functional groups in addition to the amino group and the carboxyl group may be present in the amino acid from which the amino acid moiety can be derived. A specific amino acid moiety is typically identified by the name of the amino acid from which it can be derived, e.g. as a lysine moiety, glutamic acid moiety, etc. Unless indicated otherwise in a specific context, the amino acids from which the amino acid moieties can be derived are preferably α-amino acids. If an amino acid moiety comprised by R³ or providing R³ can be derived from a chiral amino acid, preference is given to the L-configuration.

If R³ represents an amino acid moiety, it will be further understood that the amino acid moiety can be derived from an amino acid by using one of its functional groups, typically its amino group, to provide a bond, preferably an amide bond —NH—C(O)— with the carbon atom to which R³ is attached. Thus, an amino acid moiety as R³ preferably forms an amide bond with the carbon atom to which R³ is attached.

If R³ represents an amino acid moiety, the amino acid moiety is preferably derived from a hydrophilic amino acid which comprises, in addition to its amino group and its carboxyl functional group, a further hydrophilic functional group, such as a basic or acidic functional group. Such a moiety may be briefly referred to herein as “hydrophilic amino acid moiety”. For example, the further hydrophilic functional group of the hydrophilic amino acid can be selected from —NH₂, —COOH, —NH—C(═NH)—NH₂, —C(═O)NH₂, and —NH—C(═O)—NH₂. Among these, preferred are —NH₂ and —COOH.

Thus, if R³ represents an amino acid moiety, it is further preferred that the amino acid moiety is selected from a 2,3-diaminopropionic acid (Dap) moiety, 2,4-diaminobutanoic acid (Dab) moiety, ornithine (Orn) moiety, lysine (Lys) moiety, arginine (Arg) moiety, glutamic acid (Glu) moiety, aspartic acid (Asp) moiety, asparagine (Asn) moiety, glutamine (Gln) moiety, and a citrulline (Cit) moiety. Still more preferred is a moiety selected from a lysine moiety and a glutamic acid moiety, and most preferred is the lysine moiety.

If R³ represents an oligopeptide moiety, the moiety can be a linear or branched oligopeptide moiety. Preferably, the oligopeptide moiety is a moiety that can be derived from an oligopeptide which comprises or consists of 2 to 10, more preferably 2 to 5, and still more preferably 2 or 3 amino acid moieties. The amino acids providing the amino acid moieties of the oligopeptide moiety can be, independently for each occurrence, natural amino acids or synthetic amino acids. The amino acid moieties are linked via amide bonds —C(O)—NH—. This includes the possibility of an amide bond being formed via a functional group in a side chain of an amino acid. It will be further understood that the oligopeptide moiety can be derived from an oligopeptide by using one of the functional groups of its constituting amino acid moieties, typically an amino group, to provide a bond, preferably an amide bond —NH—C(O)— with the carbon atom to which R³ is attached. Thus, an oligopeptide moiety as R³ preferably forms an amide bond with the carbon atom to which R³ is attached.

Unless indicated otherwise in a specific context, one or more further functional groups in addition to the amino group and the carboxylic acid group may be present in an amino acid from which an amino acid moiety comprised by the oligopeptide can be derived. Also in this context, a specific amino acid moiety is typically identified by the name of the amino acid from which it can be derived, e.g. as a lysine moiety, glutamic acid moiety, etc. Unless indicated otherwise in a specific context, the amino acids from which the amino acid moieties can be derived are preferably α-amino acids. If an amino acid moiety constituting the oligopeptide can be derived from a chiral amino acid, preference is given to the L-configuration.

If R³ represents an oligopeptide moiety, one or more of the amino acid moieties constituting the oligopeptide moiety is/are preferably derived from a hydrophilic amino acid which comprises, in addition to its amino group and its carboxyl functional group, a further hydrophilic functional group, such as a basic or acidic functional group. As noted above, the hydrophilic amino acid may be a natural or a synthetic amino acid. Such a moiety may be briefly referred to herein as “hydrophilic amino acid moiety”. For example, the further hydrophilic functional group of the hydrophilic amino acid can be selected from —NH₂, —COOH, —NH—C(═NH)—NH₂, —C(═O)NH₂, and —NH—C(═O)—NH₂. Among these, preferred are —NH₂ and —COOH. The further hydrophilic functional group may also be used for forming a bond, typically an amide bond, to an adjacent amino acid moiety in the oligopeptide.

Thus, if R³ represents an oligopeptide moiety, it is further preferred that the one or more of the amino acid moieties constituting the oligopeptide moiety is/are selected from a 2,3-diaminopropionic acid (Dap) moiety, 2,4-diaminobutanoic acid (Dab) moiety, ornithine (Orn) moiety, lysine (Lys) moiety, arginine (Arg) moiety, glutamic acid (Glu) moiety, aspartic acid (Asp) moiety, asparagine (Asn) moiety, glutamine (Gln) moiety, and a citrulline (Cit) moiety. Still more preferred is a moiety selected from a lysine moiety and a glutamic acid moiety, and most preferred is the lysine moiety.

If R³ represents a sugar moiety or an amino sugar moiety, preferred is a moiety which can be derived from a monosaccharide, which may be a monosaccharide wherein a hydroxy group is replaced by an amino group. If R³ represents a sugar moiety or an amino sugar moiety, it will be further understood that the sugar moiety or an amino sugar moiety can be derived from the sugar or amino sugar, e.g., by using a hydroxy group of the sugar or amino sugar to provide an ester bond —O—C(O)— with the carbon atom to which R³ is attached, or by using an amino group of an amino sugar to provide an amide bond —NH—C(O)— with the carbon atom to which R³ is attached. Among the sugar moiety and amino sugar moiety, preference is given to an amino sugar moiety. The amino sugar moiety preferably forms an amide bond with the carbon atom to which R³ is attached.

Examples of a sugar moiety or an amino sugar moiety as R³ include a residue derived from 6-amino-6-deoxy-D-galactopyranose and from corresponding tautomers thereof, a residue derived from 1-amino-1-deoxy-glucopyranose, a residue derived from 1-amino-1-deoxy-galactopyranose and a residue derived from 1-amino-1-deoxy-fructopyranose.

A polyethylene glycol (PEG) moiety as R³ preferably represents a moiety comprising a group of the formula —(CH₂—CH₂—O)ₓ—, wherein X is an integer of 2 to 10, preferably 4 to 10, and is more preferably 8.

More preferably, the PEG moiety is a moiety of the formula

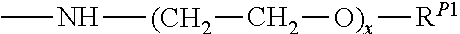

wherein the nitrogen atom providing an open bond forms an amide bond —NH—C(O)— with the carbon atom to which R³ is attached, X is an integer of 2 to 10, preferably 4 to 10, and is more preferably 8, and R^{P1} is selected from —CH₂—COOH and —CH₂—CH₂—COOH.

As an example for a combination of two or more of (ii), (iii) and (iv) which may represent R³ in line with the above, reference can be made to a combination of an amino acid moiety, which forms an amide bond with the carbon atom to which R³ is attached, and an amino sugar moiety which forms, in turn, an amide bond —NH—C(O)— with its amino group to a carboxy group of the amino acid moiety. As another example, reference can be made to a combination of an oligopeptide moiety, which forms an amide bond with the carbon atom to which R³ is attached, and an amino sugar moiety which forms, in turn, an amide bond —NH—C(O)— with its amino group to a carboxy group of the oligopeptide moiety.

Among the options for R³ discussed above, it is preferred that R³ represents a group —OH, —O⁻, or an amino acid moiety or oligopeptide moiety, more preferably an amino acid moiety or oligopeptide moiety. Among the amino acid moiety and the oligopeptide moiety, preference is given to the amino acid moiety. As discussed above, it is preferred for the amino acid moiety to be a hydrophilic amino acid moiety, and for the oligopeptide moiety to contain one or more hydrophilic amino acid moieties.

Thus, in accordance with the above, a compound of the invention is preferably a compound which comprises a group of formula (Ia) wherein R¹ and R² are each a tert-butyl group, and wherein R³ is selected from —OH, —O⁻, an amino acid moiety and an oligopeptide moiety, more preferably from an amino acid moiety and an oligopeptide moiety, or a salt of such a compound. Among the amino acid moiety and the oligopeptide moiety, preference is given to the amino acid moiety. As also discussed above, it is preferred for the amino acid moiety to be a hydrophilic amino acid moiety, and for the oligopeptide moiety to contain one or more hydrophilic amino acid moieties.

For example, preference is thus given to a compound as a compound of the invention which comprises a group of formula (Ia), wherein R⁴ and R² are each a tert-butyl group, and wherein R³ is selected from —OH, —O⁻, and a hydrophilic amino acid moiety, such as a lysine moiety, or to a salt of such a compound.

The group R^{E} in the compounds of formula (IIa), (IIb) or (IIc) or the salt thereof comprises a targeting moiety. The presence of the group R^{E} comprising the targeting moiety allows the compound of formula (IIa), (IIb) or (IIc) or the salt thereof, which further comprises a SiFA group in accordance with the invention, to bind to a target structure. Compounds of the invention or their salts can thus be used as a targeted radiopharmaceutical, in particular as a targeted radiopharmaceutical for in vivo applications in humans, such as functional molecular imaging in vivo. This includes both radiolabeled compounds of formula (IIa), (IIb) or (IIc) or a salt thereof which can be used directly as targeted radiopharmaceuticals, e.g. compounds where the SiFA group in accordance with the invention is labeled by ¹⁸F, as well as non-radiolabeled compounds which represent valuable precursors that can be radiolabeled before being used as a targeted radiopharmaceutical.

In the compounds of formula (IIa), (IIb) and (IIc) and in their salts, the targeting moiety comprised by R^{E} may be attached, e.g., directly to the carbon atom of the carbonyl group in —C(═O)R^{E} in formula (IIa), (IIb) and (IIc). In this case, R^{E} may represent the targeting moiety, i.e. the R^{E} may consist of the targeting moiety. If the targeting moiety is attached directly to the carbon atom of the carbonyl group in the substituent carrying R^{E}, it is preferred that the carbonyl group and the targeting moiety form an amide bond —C(O)—NH—, wherein the —NH— coupling group of the amide bond is provided by the targeting moiety.

Alternatively, the group R^{E} may e.g. further comprise a linker group, and the targeting moiety comprised by R^{E} may be attached via the linker group to the carbon atom of the carbonyl group in —C(═O)R^{E} in formula (IIa), (IIb) and (IIc). In the latter case, the linker group is typically attached directly (i.e. via a direct covalent bond) to the carbon atom of the carbonyl group in the substituent carrying R^{E} in formula (IIa), (IIb) and (IIc), and the targeting moiety is typically attached directly to the linker group. As will be understood by the skilled person, the presence of a linker group can be useful e.g. to facilitate the coupling of the SiFA group and the targeting moiety, or to prevent the SiFA group from interfering with the targeting function of the targeting moiety comprised by R^{E}. If such a linker group is present and is attached to the carbon atom of the carbonyl group in the substituent carrying R^{E}, is preferred that the carbonyl group and the linker group form an amide bond —C(O)—NH—, wherein the —NH— coupling group of the amide bond is provided by the linker group. Moreover, it is also preferred that the targeting moiety and the linker group comprised by R^{E} form an amide bond —C(O)—NH—.

If the linker group has a branched structure, more than one, e.g. two, targeting moieties may be attached to it. However, typically the group R^{E} comprises one targeting moiety.

Thus, the group R^{E} may consist of a linker group and a targeting moiety. In this case, the group R^{E} in formula (IIa), (IIb) and (IIc) may have the structure -L-R^{T}, wherein L is a linker group comprised by R^{E} and R^{T} is the targeting moiety comprised by R^{E}, resulting in the compounds of formulae (IIa′), (IIb′) and (IIc′) or their salts as shown below, among which further preference is given to the compounds of formula (IIa′). As will be understood, R¹, R² and R³ in these formulae are as defined above for formulae (IIa), (IIb) and (IIc), including any preferred embodiments. Also for the linker group L in formulae (IIa′), (IIb′) and (IIc′), it is preferred that the linker and the carbonyl group —C(O)— to which it is attached form an amide bond —C(O)—NH—, wherein the —NH— coupling group in the amide bond is provided by the linker L. Moreover, it is preferred that the linker L and R^{T} are attached to each other via an amide bond —C(O)—NH—. Formulae (IIa′), (IIb′) and (IIc′) represent preferred embodiments of formulae (IIa), (bib) and (IIc), respectively. Thus, unless indicated otherwise in a specific context, any reference to formulae (Ia), (bib) and (IIc) herein encompasses formulae (IIa′), (IIb′) and (IIc′).

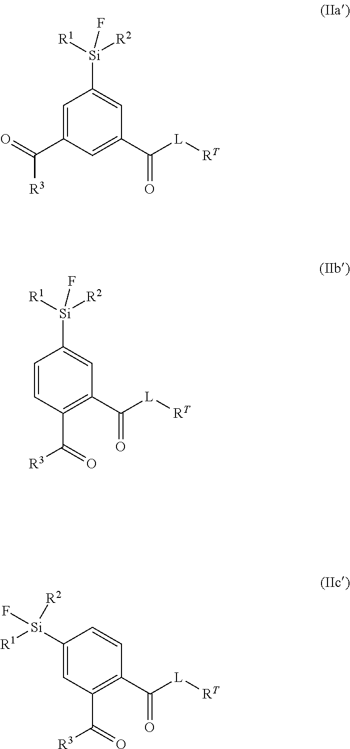

Unless indicated otherwise, the general reference to a targeting moiety comprised by R^{E} or the general reference to a targeting moiety herein relates to the targeting moiety in all of the various constellations discussed above for the compounds of formula (IIa), (IIb), and (IIc) or their salts, and the compounds of formula (IIa′), (IIb′), and (IIc′) or their salts as preferred embodiments thereof. It thus encompasses e.g. a targeting moiety which is directly attached to the carbonyl group in the substituent comprising R^{E}, or a targeting moiety which is attached via a linker group, such as the targeting moiety R^{T} in formulae (IIa′), (IIb′) and (IIc′).

The group R^{E} comprising the targeting moiety in the compounds of formula (IIa), (IIb), and (IIc) or their salts (and the group -L-R^{T} as a specific embodiment of R^{E} with the targeting moiety represented by R^{T} in the compounds of formula (IIa′), (IIb′), and (IIc′) or their salts), allows the compounds or salts to bind to a target structure. Compounds of the invention or their salts carrying the group —R^{E} or -L-R^{T} can thus be used as a targeted radiopharmaceutical, in particular as a targeted radiopharmaceutical for in vivo applications in humans, such as functional molecular imaging in vivo. A broad variety of targeting moieties are known in the field of radiopharmacy which allow a compound or salt comprising such a targeting moiety to bind to a target structure of interest, and which can be used in the context of the present invention as a targeting moiety comprised by R^{E} or as a targeting moiety R^{T}. Such targeting moieties are also referred to as targeting vectors or biological targeting moieties. It will be understood that target structures of interest are generally biological target structures, more specifically biological target structures which are associated with a disease or disorder e.g. a target structure which is overexpressed in a human disease such as cancer. Moreover, it will be understood that the compound comprising the targeting moiety generally binds preferentially to the target structure of interest, compared to other sites e.g. in the body of a patient to which the compound is administered.

A compound comprising a targeting moiety, i.e. in particular a compound of formula (IIa), (IIb), and (IIc) or a salt thereof, or a compound of formula (IIa′), (IIb′), and (IIc′) or a salt thereof, generally binds with a corresponding target structure with high affinity, e.g. as indicated by an IC50 in the low nanomolar range, preferably 50 nM or less, more preferably 10 nM or less, still more preferably 5 nM or less. The half maximal inhibitory concentration (IC50) is defined here as the quantitative measure of the molar concentration of a compound according to the invention comprising the targeting which is necessary to inhibit the binding of a radioactive reference ligand to a receptor in vitro by 50%.

As exemplary targeting moieties comprised by R^{E} reference can be made to a receptor binding moiety, an enzyme binding substrate or enzyme inhibitor, a peptide, a protein or an antibody fragment or an engineered antigen binding construct, such as a nanobody. Among these exemplary moieties, preference is given to a targeting moiety selected from a receptor binding moiety, an enzyme binding substrate and an enzyme inhibitor.

For example, the targeting moiety comprised by R^{E} may be a peptidic targeting moiety, i.e. a moiety constituted by amino acid units, such as 2 to 20 amino acid units, or preferably 2 to 10 amino acid units. Peptides suitable to provide a peptidic targeting moiety include linear and cyclic peptides, or peptides combining a linear and a cyclic portion.

Typically, if the targeting moiety is a receptor binding moiety as referred to above, the presence of the targeting moiety in the compound of the invention or its salt allows the compound or salt to act as a ligand for the concerned receptor. Likewise, if the targeting moiety is a moiety binding to prostate-specific membrane antigen (PSMA binding moiety) as referred to above, the presence of the targeting moiety in the compound of the invention or its salt, allows the compound or salt to act as a ligand for PSMA.

If the targeting moiety comprised by R^{E} represents a receptor binding moiety or an enzyme binding substrate in line with the above, the compound of the invention comprising the targeting moiety is generally capable of binding with high affinity to a receptor or to an enzyme, respectively. In this context, high affinity binding preferably means that the compound comprising the receptor binding moiety or the enzyme binding substrate exhibits an IC50 in the low nanomolar range, preferably 50 nM or less, more preferably 10 nM or less, still more preferably 5 nM or less. For the sake of clarity, the half maximal inhibitory concentration (IC50) is defined here as the quantitative measure of the molar concentration of a compound according to the invention comprising the receptor binding moiety or the enzyme binding substrate, respectively, which is necessary to inhibit the binding of a radioactive reference ligand to a receptor in vitro by 50%.

As exemplary receptors as target structures of interest for the targeting moiety comprised by R^{E}, mention may be made of a gastrin releasing peptide receptor (GRPR), a C-X-C chemokine receptor type 4 (CXCR4), a somatostatin receptor (SSTR), a cholecystokinin B receptor (CCK-2R) and prostate-specific membrane antigen (PSMA). Thus, exemplary receptor binding moieties as a targeting moiety include a gastrin releasing peptide receptor (GRPR) binding moiety, a C-X-C chemokine receptor type 4 (CXCR4) binding moiety, a somatostatin receptor (SSTR) binding moiety, or a cholecystokinin B receptor (CCK-2R) binding moiety.

As examples for a SSTR binding moiety suitable as a targeting moiety comprised by R^{E}, mention may be made of a moiety that can be derived from a receptor agonist or receptor antagonist selected from Tyr³-Octreotate (or Tyr³,Thr⁸-Octreotide, TATE, H-D-Phe-cyclo(L-Cys-L-Tyr-D-Trp-L-Lys-L-Thr-L-Cys)-L-Thr-OH), Thr⁸-Octreotide (ATE), Phe¹,Tyr³-Octreotide (TOC, H-D-Phe-cyclo(L-Cys-L-Tyr-D-Trp-L-Lys-L-Thr-L-Cys)-L-Thr-ol), NaI³-Octreotide (NOC, H-D-Phe-cyclo(L-Cys-L-1-NaI-D-Trp-L-Lys-L-Thr-L-Cys)-L-Thr-ol), 1-NaI³,Thr⁸-Octreotide (NOCATE), BzThi³-Octreotide (BOC), BzThi³,Thr⁸-Octreotide (BOCATE), JR11 (H-L-Cpa-cyclo(D-Cys-L-Aph(Hor)-D-Aph(Cbm)-L-Lys-L-Thr-L-Cys)-D-Tyr-NH₂), BASS (H-L-Phe(4-NO₂)-cyclo(D-Cys-L-Tyr-D-Trp-L-Lys-L-Thr-L-Cys)-D-Tyr-NH₂) and KE121 (cyclo(D-Dab-L-Arg-L-Phe-L-Phe-D-Trp-L-Lys-L-Thr-L-Phe)), more preferably from TATE or JR11, and most preferably from TATE. As will be understood by the skilled reader, a receptor binding moiety as targeting moiety can be conveniently derived from the exemplary receptor agonists or antagonists listed above by using a functional group, such as a carboxyl group or an amino group, contained in the receptor agonist or antagonist, to provide a coupling group which attaches the targeting moiety to the remainder of the compound of the invention. Preferably, these receptor agonists or receptor antagonists provide a targeting moiety by using an amino group contained therein, e.g. in an optionally substituted phenylalanine unit, to form an amide bond —C(O)—NH— with the remainder of the compound of the invention.

Specifically, reference may be made to a moiety of one of the following formulae as an exemplary SSTR binding moiety suitable as a targeting moiety, wherein the dashed line marks the bond which attaches the group to the remainder of the compound of the invention:

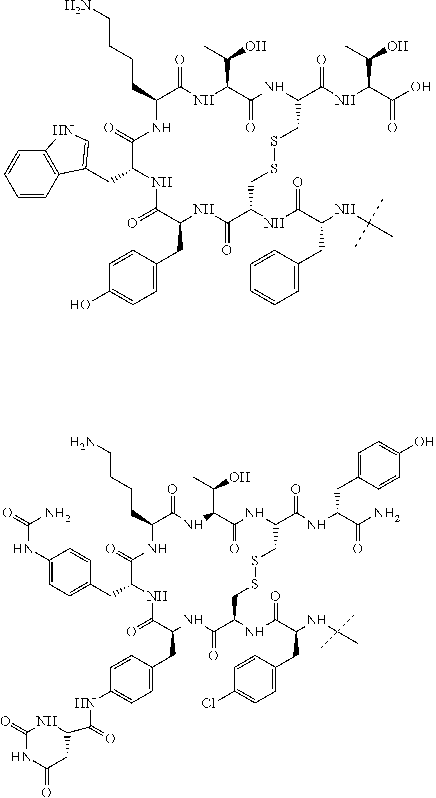

As an exemplary CXCR4 binding moiety suitable as a targeting moiety comprised by R^{E}, reference may be made to a moiety of the following formula, wherein R^{B1} is H or I, and the dashed line marks the bond which attaches the group to the remainder of the compound of the invention:

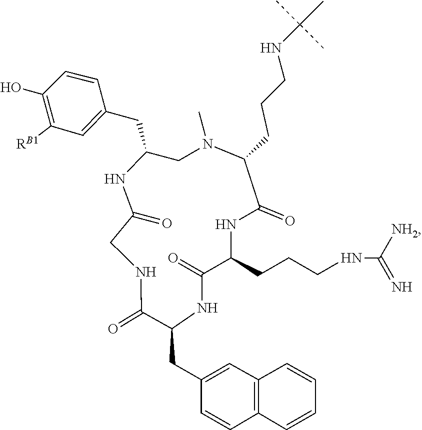

As an exemplary PSMA binding moiety suitable as a targeting moiety comprised by R^{E}, reference may be made to a moiety of the following formula:

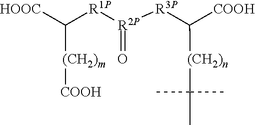

wherein:
m is an integer of 2 to 6, preferably 2 to 4, more preferably 2;
n is an integer of 1 to 6, preferably 2 to 4, more preferably 2 or 4;
R^{1P} is CH₂, NH or O, preferably NH;
R^{3P} is CH₂, NH or O, preferably NH;
R^{2P} is C or P(OH), preferably C;
and wherein the dashed line marks the bond which attaches the group to the remainder of the compound of the invention. Typically, the PSMA binding moiety is attached to the remainder of the compound of the invention via an amide bond —NH—C(O)—, and it is thus preferred that a group —NH— or a group —C(O)—, more preferably a group —NH—, is further provided at the position marked by the dashed line in the above formula, which group forms a part of such an amide bond. More preferably, the PSMA binding moiety is a moiety of the following formula:

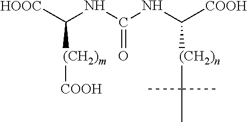

wherein:
m is an integer of 2 to 6, preferably 2 to 4, more preferably 2;
n is an integer of 1 to 6, preferably 2 to 4, more preferably 2 or 4;
and wherein the dashed line marks the bond which attaches the group to the remainder of the compound of the invention. Typically, the more preferred PSMA binding moiety is attached to the remainder of the compound of the invention via an amide bond —NH—C(O)—, and it is thus preferred that a group —NH— or a group —C(O)—, more preferably a group —NH—, is further provided at the position marked by the dashed line in the above formula, which group forms a part of such an amide bond.

As exemplary GRPR binding moieties suitable as a targeting moiety comprised by R^{E}, reference may be made to the targeting moieties comprised by the modified GRPR antagonist peptides disclosed in WO 2021/121735.

As a particularly preferred example of a GRPR binding moiety suitable as a targeting moiety comprised by R^{E}, reference can be made to the following group comprised in the GRPR antagonist RM2, i.e. -Pip-D-Phe-L-Gln-L-Trp-L-Ala-L-Val-Gly-L-His-L-Sta-L-Leu-NH₂,

wherein Pip denotes a 4-amino-1-carboxymethyl-piperidine residue, with the carboxyl group forming an amide bond with D-Phe.

As exemplary CCK-2R binding moieties suitable as a targeting moiety comprised by R^{E}, reference may be made to the peptidomimetic amino acid polymers disclosed in WO 2018/224665.

As particularly preferred examples of a CCK-2R binding moiety suitable as a targeting moiety comprised by R^{E}, reference can be made to the peptidic group comprised in the ligand compounds PP-F11 or PP-F11N, i.e.

-D-Glu-D-Glu-D-Glu-D-Glu-D-Glu-D-Glu-L-Ala-
L-Tyr-Gly-L-Trp-L-Met-L-Asp-L-Phe-NH₂
and
-D-Glu-D-Glu-D-Glu-D-Glu-D-Glu-D-Glu-L-Ala-
L-Tyr-Gly-L-Trp-L-Nie-L-Asp-L-Phe-NH₂,
and to
-D-Glu-L-Ala-L-Tyr-Gly-L-Trp-L-(N-Me)Nie-
L-Asp-L-1Nal-NH₂.

In addition to the targeting moiety, the group R^{E} may comprise a chelating moiety or a chelate moiety formed by the chelating moiety and a chelated metal cation. If present, the chelating/chelate moiety typically forms part of a linker that may be comprised by R^{E}, such as the linker L referred to above. More than one, e.g. two, chelating/chelate moieties may form part of such a linker. A chelating/chelate moiety may be part of a linear linker structure, or may be part of a branched linker structure, e.g. by providing a branch thereof.

The chelating moiety is preferably a moiety which can be derived from a chelating agent selected from diethylenetriaminepentamethylenephosphonic acid (EDTMP) and its derivatives, diethylenetriaminepentaacetic acid (DTPA) and its derivatives, bis(carboxymethyl)-1,4,8,11-tetraaza-bicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino],pentyl]-N-[5-[[4-[5-aminopentyl-(hydroxy)amino]-4-oxobutanoyl]-amino]pentyl]-N-hydroxybutandiamide (DFO) and derivatives thereof, 1,4,7,10-tetraazacyclododecane-1,7-diacetic acid (DO2A), 1,4,7,10-tetraazacyclododecan-N,N′,N″,N′″-tetraacetic acid (DOTA), 2-[1,4,7,10-tetraazacyclododecane-4,7,10-triacetic acid]-pentanedioic acid (DOTAGA or DOTA-GA), 1,4,7,10-tetrakis(carbamoylmethyl)-1,4,7,10-tetraazacyclododecane (DOTAM), N,N′-dipyridoxylethylendiamine-N,N′-diacetate-5,5′-bis(phosphat) (DPDP), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 1,4,7-triazacyclononan-1-succinic acid-4,7-diacetic acid (NODASA), 1-(1-carboxy-3-carboxypropyl)-4,7-(carboxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetra¬acetic acid (TETA), terpyridine-bis(methyleneamine) tetraacetic acid (TMT), 1,4,7,10-tetraazacyclotridecan-N,N′,N″,N′″-tetraacetic acid (TRITA), and triethylenetetraaminehexaacetic acid (TTHA), N,N′-bis[(6-carboxy-2-pyridyl)methyl]-4,13-diaza-18-crown-6 (H₂macropa), 4-amino-4-{2-[(3-hydroxy-1,6-dimethyl-4-oxo-1,4-dihydro-pyridin-2-ylmethyl)-carbamoyl]-ethyl}heptanedioic acid bis-[(3-hydroxy-1,6-dimethyl-4-oxo-1,4-dihydro-pyridin-2-ylmethyl)-amide](THP), 1,4,7-triazacyclononane-1,4,7-tris[methylene(2-carboxyethyl)phosphinic acid (TRAP), 2-(4,7,10-tris(2-amino-2-oxoethyl)-1,4,7,10-tetraazacyclododecan-1-yl)acetic acid (DO3AM), and 1,4,7,10-tetraazacyclododecane-1,4,7,10-tetrakis[methylene(2-carboxyethylphosphinic acid)](DOTPI), S-2-(4-isothiocyanatobenzyl)-1,4,7,10-tetraazacyclododecane tetraacetic acid, hydrazinonicotinic acid (HYNIC), 1-N-(4-aminobenzyl)-3,6,10,13,16,19-hexaazabicyclo[6.6.6]-eicosan-1,8-diamine (SarAr), 6-Amino-6-methylperhydro-1,4-diazepine-N,N,N′,N′-tetraacetic acid (AAZTA) and derivatives thereof, such as (6-pentanoic acid)-6-(amino)methyl-1,4-diazepine triacetate (DATA), pentadeca-1,4,7,10,13-penta-aminopentaacetic acid (PEPA), hexadeca-1,4,7,10,13,16-hexaamine-hexaacetic acid (HEHR), 4-{[bis(phosphonomethyl)) carbamoyl]methyl}-7,10-bis (carboxymethyl)-1,4,7,10-tetraazacyclododec-1-yl) acetic acid (BPAMD), N (4-{[bis(phosphonomethyl)) carbamoyl]methyl}-7,10-bis(carboxymethyl)-nona-1,4,7-triamine triacetic acid (BPAM), 1,2-[{6-(carboxylate) pyridin-2-yl}methylamine]ethane (DEDPA, H₂DEDPA), deferoxamine (DFO) and its derivatives, deferiprone, (4-acetylamino-4-yl) {2-[(3-hydroxy-1,6-dimethyl-4-oxo-1,4-dihydro-pyridin-2-ylmethyl)-carbamoyl]-ethyl}-heptanedioic acid bis-[(3-hydroxy-1,6-dimethyl-4-oxo-1,4-dihydro-pyridin-2-ylmethyl)-amide](CP256) and its derivatives such as YM103; tetraazycyclodecane-phosphinic acid (TEAP), 6,6′-[{9-hydroxy-1,5-bis-(methoxycarbonyl)-2,4-di(pyridin-2-yl)-3,7-diazabicyclo[3.3.1]nonane-3,7-diyl}bis(methylene)]dipicolinic acid (H₂bispa²), 1,2-[{6-(carboxylato)pyridin-2-yl}methylamino]-ethane (H₂dedpa), N,N′-bis(6-carboxy-2-pyridylmethyl)-ethylenediamine-N,N′-diacetic acid (H₄octapa), N,N′-bis(2-hydroxy-5-sulfonylbenzyl)-N,N′-bis-(2-methylpyridyl)ethylenediamine (H₆Sbbpen) and derivatives thereof, triethylenetetramine-N,N,N′,N″,N′″,N′″-hexaacetic (TTHA), 2-aminomethylpiperidine triacetic acid (2-AMPTA) and derivatives thereof, such as 2-(N-(2-Hydroxybenzyl)aminomethyl)piperidine (2-AMPTA-HB) further functionalized derivative of 2-AMPTA with additional functional groups suitable for conjugation to peptidic structures, 4-nitro-2-hydroxybenzyl-2-{[(6)-trans-2-[benzyl(carboxymethyl)amino]cyclohexyl](carboxymethyl)amino}acetic acid (RESCA) and derivatives thereof, and 6-carboxy-1,4,8,11-tetraazaundecane (N₄) and derivatives thereof. Among these chelating agents, preference is given to DOTA, DOTAGA, DOTAM, DO3AM, NOTA and NODAGA. Most preferred are DOTA and DOTAGA.

As will be understood by the skilled reader, preferred chelate moieties are formed by the preferred chelating moieties discussed above and a chelated metal cation.

As will further be understood by the skilled reader, a chelating moiety or a chelate moiety in a compound in accordance with the invention can be conveniently derived from the chelating agents listed above by using at least one, e.g. one or two, functional groups contained in the chelating agent, such as a carboxyl group, a carboxamide group, an amino group, or a hydroxy group, to provide a coupling group, e.g. selected from —C(O)—, —NH—, and —O—, which attach(es) the chelating moiety to the remainder of the compound. Preferably, a carboxyl group is used to provide a coupling group —C(O)— to attach the chelating moiety or chelate moiety via an amide bond —C(O)—NH—.

The metal cation that may be chelated in the chelate moiety may be radioactive or non-radioactive metal cation.

The metal cation that may be chelated in the chelate moiety is preferably selected from cations of ⁴³Sc, ⁴⁴Sc, ⁴⁷Sc, ⁵¹Cr, ^{52m}Mn, ⁵⁵Co, ⁵⁷Co, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ^{94m}Tc, ^{99m}Tc, ⁹⁷Ru, ¹⁰⁵Rh, ¹⁰⁹Pd, ¹¹¹Ag, ^{110m}In, ¹¹¹In, ^{113m}In, ^{114m}In, ^{11m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁵⁵Tb, ¹⁶¹Tb, ¹⁶⁴Tb, ¹⁶¹Ho, ¹⁶⁶Ho, ¹⁵⁷Dy, ¹⁶⁵Dy, ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ¹⁷⁷Lu, ¹⁸⁶Re, ^{186g}Re, ¹⁸⁸Re, ¹⁸⁸W, ¹⁹¹Pt, ^{195m}Pt, ¹⁹⁴Ir, ¹⁹⁷Hg, ¹⁹⁸Au, ¹⁹⁹Au, ²¹²Pb, ²⁰³Pb, ²¹¹At, ²¹²Bi, ²¹³Bi, ²²³Ra, ²²⁴Ra, ²²⁵Ac, ²²⁶Th and ²²⁷Th, and from nonradioactive isotopes of any of these metals. The chelated cation may be a complex cation, e.g. a metal ion carrying an additional coordinated ligand other than the chelating group, such as an oxo-ligand in a chelate including a ^{99m}Tc(V)-oxo core.

Particularly preferred as a chelated cation is a radioactive or non-radioactive cation of Ga or Lu, such as ¹⁷⁷Lu or ⁶⁸Ga.

In line with the above, if the compound of the invention comprises a chelating moiety or a chelate moiety formed by the chelating moiety and a chelated radioactive or nonradioactive metal cation, it is preferred that the chelating moiety is derived from DOTA or DOTAGA, typically derived by using a carboxyl group comprised by DOTA or DOTAGA to provide a coupling group —C(O)— to attach the chelating moiety to the remainder of the compound, and that a chelated cation of the chelate moiety is a cation of Ga or Lu and which may be radioactive, such as a cation of ¹⁷⁷Lu or ⁶⁸Ga, or which may be non-radioactive.

In line with the above, a mandatory component of the group R^{E} in the compounds of formula (IIa), (IIb), (IIc) and their salts is the targeting moiety, which is represented by the group R^{T} in the compounds of formula (IIa′), (IIb′) and (IIc′) and their salts. An optional component of the group R^{E} in the compounds of formula (IIa), (IIb), (IIc) and their salts is a linker. Such a linker is preferably comprised by R^{E}, e.g. as L in the preferred compounds of formula (IIa′), (IIb′), or (IIc′) and their salts wherein —R^{E} is represented by -L-R^{T}. Another optional component of the group R^{E} is a chelating moiety or a chelate moiety formed by the chelating moiety and a chelated radioactive or nonradioactive metal cation. If a chelating moiety/chelate moiety is comprised by R^{E}, a linker is typically also comprised by R^{E}, such as the linker L, and the chelating moiety/chelate moiety forms part of the linker.

A linker which may be comprised by R^{E} in formulae (IIa), (IIb) and (IIc) as discussed above, such as a linker L in formulae (IIa′), (IIb′) and (IIc′), may have e.g. a linear or a branched backbone structure. The linker may comprise one or more moieties which provide an additional function in the compounds of the invention, such as a chelating moiety or a chelate moiety which is formed by the chelating moiety and a chelated metal cation. Such moieties having an additional function may e.g. be incorporated into a branched or linear backbone of the linker.

As examples of a moiety suitable as a linker, mention may be made of an amino acid moiety or an oligoamide moiety. As noted above, such an amino acid moiety or oligoamide moiety as a linker may optionally comprise one or more further moieties which provide an additional function in the compounds of the invention, such as a chelating moiety or a chelate moiety which is formed by the chelating moiety and a chelated metal cation.

For example, an amino acid moiety acting as a linker may be a difunctional moiety which provides a coupling group —NH—e.g. for forming an amide bond with the carbonyl group to which R^{E} is attached, and a coupling group —C(O)—e.g. for forming an amide bond with the targeting moiety comprised by R^{E}, such as R^{T}, wherein the —NH— coupling group in the amide bond is provided by the targeting moiety. As another example, the amino acid moiety may be a trifunctional moiety, which provides a coupling group —NH—e.g. for forming an amide bond with the carbonyl group to which R^{E} is attached, a coupling group —C(O)—e.g. for forming an amide bond with the targeting moiety comprised by R^{E}, and a further functional group, such as a further carboxy group or a further amino group.

For an oligoamide moiety as an exemplary linker comprised by R^{E}, it is likewise preferred that the linker and the carbonyl group —C(O)— to which R^{E} is attached form an amide bond —C(O)—NH—, wherein the —NH— coupling group in the amide bond is provided by the oligoamide moiety. Moreover, it is preferred that the oligoamide moiety and the targeting moiety comprised by R^{E}, such as R^{T}, form an amide bond —C(O)—NH—.

As will be understood by the skilled person, an oligoamide moiety comprises two or more, such as 2 to 10, preferably 2 to 8, and more preferably 2 to 6 subunits which are linked to each other via amide bonds —C(O)—NH—. For example, each of the subunits may be formed by 7 to 30 atoms. If one of the subunits provides more than two, such as three, coupling groups selected from —C(O)— and —NH— which are suitable to form an amide bond —C(O)—NH—, the oligoamide moiety may be a branched moiety.

As will be understood by the skilled person, subunits suitable for a linear or branched oligoamide linker may for example be selected from
a unit providing a coupling group —NH— and a coupling group —C(O)—, which unit may be derived e.g. from an amino acid,
a unit providing two coupling groups —NH—, which unit may be derived e.g. from a diamino compound, or from a compound containing two amino groups in combination with one or more further functional groups, an example being an amino acid which comprises a side chain carrying an additional terminal amino group,
a unit providing two coupling groups —C(O)—, which unit may be derived e.g. from a dicarboxylic acid, or from a compound containing two carboxy groups in combination with one or more further functional groups, examples being an amino acid which comprises a side chain carrying an additional terminal carboxy group, and a chelating agent which comprises two, three or four carboxy groups or carboxamide groups of which two provide a coupling group —C(O)—;
a unit providing two coupling groups —NH— and one coupling group —C(O)—, which unit may be derived e.g. from an amino acid which comprises a side chain carrying a terminal amino group, and
a unit providing two coupling groups —C(O)— and one coupling group —NH—, which unit may be derived e.g. from an amino acid which comprises a side chain carrying a terminal carboxy group.

An oligoamide moiety having a branched structure acting as a linker L will typically contain a terminal subunit which forms only one amide bond —C(O)—NH— with an adjacent unit. As an example, mention may be made of a unit derived from a chelating agent which comprises two, three or four carboxy groups or carboxamide groups of which one provides a coupling group —C(O)—.

For example, and in line with the above with the above, a preferred compound of the invention is therefore a compound of formula (IIa) or of formula (IIa′) wherein R^{E}, L and R^{T} are respectively defined as above, including any preferred embodiments of their definitions, and wherein R¹ and R² are each a tert-butyl group, and R³ is selected from —OH, —O⁻, an amino acid moiety and an oligopeptide moiety, more preferably from an amino acid moiety and an oligopeptide moiety, or a salt of such a compound. Among the amino acid moiety and the oligopeptide moiety, preference is given to the amino acid moiety. As also discussed above, it is preferred for the amino acid moiety to be a hydrophilic amino acid moiety, and for the oligopeptide moiety to contain one or more hydrophilic amino acid moieties.

Further exemplary compounds of formula (IIa), (IIb) and (IIc) and salts thereof in accordance with the invention can be illustrated by the following formulae (IIIa) and their salts:

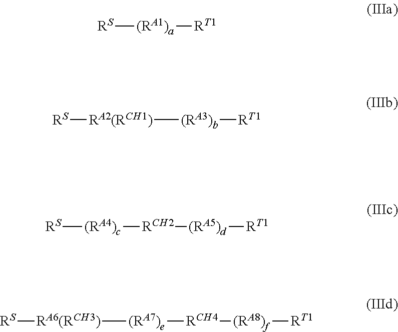

In these formulae, R^{S} represents a group of formula (Ia), (Ib) or (Ic) as discussed herein, preferably of formula (Ia). R^{T1} represents a targeting moiety as discussed herein, such as a gastrin releasing peptide receptor (GRPR) binding moiety, a C-X-C chemokine receptor type 4 (CXCR4) binding moiety, a somatostatin receptor (SSTR) binding moiety, a cholecystokinin B receptor (CCK-2R) binding moiety, or a PSMA binding moiety, including the examples for these binding moieties disclosed herein.

For example, and in line with the above, a preferred example of the compounds of formula (IIIa) to (IIId) is a compound wherein R^{S} is a group of formula (Ia) wherein R¹ and R² are each a tert-butyl group, and wherein R³ is selected from —OH, —O⁻, an amino acid moiety and an oligopeptide moiety, more preferably from an amino acid moiety and an oligopeptide moiety, or a salt of such a compound. Among the amino acid moiety and the oligopeptide moiety, preference is given to the amino acid moiety. As also discussed above, it is preferred for the amino acid moiety to be a hydrophilic amino acid moiety, and for the oligopeptide moiety to contain one or more hydrophilic amino acid moieties.

Moreover, in formula (IIIa):

The group R^{A1} represents, independently for each occurrence if more than one group R^{A1} is present, a divalent group which forms amide bonds —NH—C(O)— with two adjacent groups (e.g. R^{S}, another group R^{A1}, or R^{T1}). Preferably, R^{A1} contains 2 to 8 carbon atoms, not including any carbonyl carbon atoms involved in an amide bond. It will be understood by the skilled reader that each group R^{A1} can be independently selected among compounds providing functional groups suitable for the formation of amide bonds, taking due account of the desired arrangement of the groups/moieties contained in the compound of formula (Illa). For example, a group R^{A1} can be a group providing a coupling group —NH— and a coupling group —C(O)—, a group providing two coupling groups —NH—, or a group providing two coupling groups —C(O)—.

The variable a represents an integer from 0 to 4, such as 0, 1, 2 or 3.

In formula (IIIb):

R^{CH1} represents a chelating moiety or a chelate moiety which is formed by the chelating moiety and a chelated metal cation as discussed herein, including the examples for the chelating moiety disclosed herein, such as a moiety derived from DOTA or DOTAGA. As will be understood from formula (IIIb), R^{CH1} is a monovalent moiety which is attached to R^{A2}. R^{CH1} and R^{A2} are bound to each other via an amide bond —NH—C(O)—.

The group R^{A2} represents a trivalent group which forms amide bonds —NH—C(O)— with R^{S} and R^{CH1} and, if present, R^{A3} or R^{T1}. Preferably, R^{A2} contains 2 to 8 carbon atoms, not including any carbonyl carbon atoms involved in an amide bond. It will be understood by the skilled reader that R^{A2} can selected among compounds providing functional groups suitable for the formation of amide bonds, taking due account of the desired arrangement of the groups/moieties contained in the compound of formula (IIIb). For example, a group R^{A2} can be a group providing two coupling groups —NH— and a coupling group —C(O)—.

The group R^{A3} represents, independently for each occurrence if more than one group R^{A3} is present, a divalent group which forms amide bonds —NH—C(O)— with R^{A2} and, if present, another group R^{A3}, or R^{T1}. Preferably, R^{A3} contains 2 to 8 carbon atoms, not including any carbonyl carbon atoms involved in an amide bond. It will be understood by the skilled reader that each group R^{A3} can be independently selected among compounds providing functional groups suitable for the formation of amide bonds, taking due account of the desired arrangement of the groups/moieties contained in the compound of formula (IIIb). For example, a group R^{A3} can be a group providing a coupling group —NH— and a coupling group —C(O)—, a group providing two coupling groups —NH—, or a group providing two coupling groups —C(O)—.

The variable b represents an integer from 0 to 4, such as 0, 1, 2 or 3.

In formula (IIIc):

The group R^{A4} represents, independently for each occurrence if more than one group R^{A4} is present, a divalent group which forms amide bonds —NH—C(O)— with R^{S} and R^{CH2}. Preferably, R^{A4}contains 2 to 8 carbon atoms, not including any carbonyl carbon atoms involved in an amide bond. It will be understood by the skilled reader that each group R^{A4} can be independently selected among compounds providing functional groups suitable for the formation of amide bonds, taking due account of the desired arrangement of the groups/moieties contained in the compound of formula (IIIc). For example, a group R^{A4} can be a group providing a coupling group —NH— and a coupling group —C(O)—, a group providing two coupling groups —NH—, or a group providing two coupling groups —C(O)—.

The variable c represents an integer from 1 to 4, such as 1, 2 or 3, preferably 1 or 2.

R^{CH2} represents a chelating moiety or a chelate moiety which is formed by the chelating moiety and a chelated metal cation as discussed herein, including the examples for the chelating moiety disclosed herein, such as a moiety derived from DOTA or DOTAGA. As will be understood from formula (IIIc), R^{CH2} is a divalent moiety which is attached to R^{A4} and, if present, R^{A5} or R^{T1}. R^{CH2} is bound to its adjacent groups via an amide bond —NH—C(O)—.

The group R^{A5} represents, independently for each occurrence if more than one group R^{A5} is present, a divalent group which forms amide bonds —NH—C(O)— with R^{CH2} and R^{T1}. Preferably, R^{A5}contains 2 to 8 carbon atoms, not including any carbonyl carbon atoms involved in an amide bond.

It will be understood by the skilled reader that each group R^{A5} can be independently selected among compounds providing functional groups suitable for the formation of amide bonds, taking due account of the desired arrangement of the groups/moieties contained in the compound of formula (IIIc). For example, a group R^{A5} can be a group providing a coupling group —NH— and a coupling group —C(O)—, a group providing two coupling groups —NH—, or a group providing two coupling groups —C(O)—.

The variable d represents an integer from 0 to 4, such as 0, 1, 2 or 3, preferably 0, 1 or 2.

In formula (IIId):

R^{CH3} represents a chelating moiety or a chelate moiety which is formed by the chelating moiety and a chelated metal cation as discussed herein, including the examples for the chelating moiety disclosed herein, such as a moiety derived from DOTA or DOTAGA. As will be understood from formula (IIId), R^{CH3} is a monovalent moiety which is attached to R^{A4}. R^{CH3} and R^{A4} are bound to each other via an amide bond —NH—C(O)—.

The group R^{A6} represents a trivalent group which forms amide bonds —NH—C(O)— with R^{S}, R^{CH3} and R^{A7}. Preferably, R^{A6} contains 2 to 8 carbon atoms, not including any carbonyl carbon atoms involved in an amide bond. It will be understood by the skilled reader that R^{A6} can selected among compounds providing functional groups suitable for the formation of amide bonds, taking due account of the desired arrangement of the groups/moieties contained in the compound of formula (IIId). For example, a group R^{A6} can be a group providing two coupling groups —NH— and a coupling group —C(O)—.

The group R^{A7} represents, independently for each occurrence if more than one group R^{A7} is present, a divalent group which forms amide bonds —NH—C(O)— with R^{A6} and R^{CH4}. Preferably, R^{A7}contains 2 to 8 carbon atoms, not including any carbonyl carbon atoms involved in an amide bond. It will be understood by the skilled reader that each group R^{A7} can be independently selected among compounds providing functional groups suitable for the formation of amide bonds, taking due account of the desired arrangement of the groups/moieties contained in the compound of formula (IIId). For example, a group R^{A7} can be a group providing a coupling group —NH— and a coupling group —C(O)—, a group providing two coupling groups —NH—, or a group providing two coupling groups —C(O)—.

The variable e represents an integer from 1 to 4, such as 1, 2 or 3, preferably 1 or 2.

R^{CH4} represents a chelating moiety or a chelate moiety which is formed by the chelating moiety and a chelated metal cation as discussed herein, including the examples for the chelating moiety disclosed herein, such as a moiety derived from DOTA or DOTAGA. As will be understood from formula (IIId), R^{CH4} is a divalent moiety which is attached to R^{A7} and, if present, R^{A8} or R^{T1}. R^{CH4} is bound to its adjacent groups via an amide bond —NH—C(O)—.

The group R^{A8} represents, independently for each occurrence if more than one group R^{A}e is present, a divalent group which forms amide bonds —NH—C(O)— with R^{CH4} and R^{T1}. Preferably, R^{A8} contains 2 to 8 carbon atoms, not including any carbonyl carbon atoms involved in an amide bond. It will be understood by the skilled reader that each group R^{A}a can be independently selected among compounds providing functional groups suitable for the formation of amide bonds, taking due account of the desired arrangement of the groups/moieties contained in the compound of formula (IIId). For example, a group R^{A8} can be a group providing a coupling group —NH— and a coupling group —C(O)—, a group providing two coupling groups —NH—, or a group providing two coupling groups —C(O)—.

The variable f represents an integer from 0 to 4, such as 0, 1, 2 or 3, preferably 0, 1 or 2.

As noted above, the compounds in accordance with the invention encompass compounds comprising a group selected from a group of formula (Ia), (Ib) and (Ic), or a salt thereof, and compounds of formula (IIa), (IIb) or (IIc), inclusive of formulae (IIa′) to (IIIc′) and (IIIa) to (IIId), or a salt of the compounds. Salts are preferably pharmaceutically acceptable salts, i.e. formed with pharmaceutically acceptable anions or cations. Salts may be formed, e.g., by protonation of an atom carrying an electron lone pair which is susceptible to protonation, such as a nitrogen atom, with an inorganic or organic acid, or by separating a proton from an acidic group, such as a carboxy group, e.g. by neutralization with a base. Other charged groups which may be present in the compounds in accordance with the invention and which may provide the compounds in the form of a salt include groups which are continuously charged, such as a quaternary ammonium group comprising an ammonium cation wherein the nitrogen is substituted by four organyl groups, or charged chelate complexes.

As exemplary anions which may be present as counterions in salt forms of the compounds of the invention if the salt form comprises a positively charged form of the compound, mention may be made, for example, of an anion selected from chloride, bromide, iodide, sulfate, nitrate, phosphate (such as, e.g., phosphate, hydrogenphosphate, or dihydrogenphosphate salts), carbonate, hydrogencarbonate or perchlorate; acetate, trifluoroacetate, propionate, butyrate, pentanoate, hexanoate, heptanoate, octanoate, cyclopentanepropionate, undecanoate, lactate, maleate, oxalate, fumarate, tartrate, malate, citrate, nicotinate, benzoate, salicylate or ascorbate; sulfonates such as methanesulfonate, ethanesulfonate, 2-hydroxyethanesulfonate, benzenesulfonate, p-toluenesulfonate (tosylate), 2-naphthalenesulfonate, 3-phenylsulfonate, or camphorsulfonate. Since trifluoroacetic acid is frequently used during the synthesis of peptides, trifluoroacetate salts are typical salts which are provided if a compound comprising a peptide structure is formed. Such trifluoroacetate salts may be converted e.g. to acetate salts during their workup.

As exemplary cations which may be present as counterions in salt forms of the compounds of the invention if the salt form comprises a negatively charged form of the compound, mention may be made, for example, of a cation selected from alkali metal cations, such as lithium, sodium or potassium, alkaline earth metal cations, such as calcium or magnesium; and ammonium (including ammonium ions substituted by organic groups).

The compounds in accordance with the invention comprise a SiFA group wherein a fluorine atom F attached via a direct covalent bond to a Si atom as shown in formulae (Ia), (Ib), (Ic), (IIa), (IIb) and (IIc). As will be understood, the F may be ¹⁸F (i.e. a radioactive fluorine isotope) or ¹⁹F (a non-radioactive fluorine isotope also referred to as “cold fluorine”).

As noted above, the SiFA groups in accordance with the invention allow a fast and efficient isotopic exchange reaction between ¹⁹F and ¹⁸F to be accomplished. Thus, the invention further provides a method for the preparation of a radiolabeled compound, comprising a step of reacting a compound comprising a group selected from a group of formula (Ia), (Ib) and (Ic), or a salt thereof, or a compound of formula (IIa), (IIb) or (IIc) or a salt thereof, wherein the fluorine attached via a direct covalent bond to the Si atom is [¹⁹F]fluorine, with [¹⁸F]fluoride to exchange the [¹⁹F]fluorine by [¹⁸F]fluorine. Sources of [¹⁸F]fluoride and setups which can be used for the isotopic exchange of ¹⁹F to ¹⁸F in SiFA groups are known to the skilled person and can be relied on for the method of the invention as described herein. Reference can be made, e.g., to C. Wangler et al., Appl. Sci. 2012, 2(2), 277-302 (https://doi.org/10.3390/app2020277) or A. Wurzer et al., EJNMMI Radiopharm. Chem. 6, 4 (2021) (https://doi.org/10.1186/s41181-021-00120-5). It will be appreciated that the information provided above with respect to preferred embodiments of the groups of formula (Ia), (Ib) and (Ic) or the compounds of formula (IIa), (IIb) or (IIc) continues to apply in the context of this method.

To that extent, a further aspect of the invention is the use of a group of the formula (Ia), (Ib) or (Ic) as defined above, including any preferred embodiments thereof, as a silicon-based fluoride acceptor group for the isotopic exchange of [¹⁹F]fluorine by [¹⁸F]fluorine.

A further aspect of the invention is the use of a group of the formula (Ia), (Ib) or (Ic) as defined above, including any preferred embodiments thereof, as a silicon-based fluoride acceptor group for the [¹⁸F]labeling of a targeted radiopharmaceutical, e.g. a targeted radiopharmaceutical which is a compound of the formula (IIa), (IIb) or (IIc) or a salt thereof as defined above, including any preferred embodiments thereof, comprising [¹⁸F]fluorine as the fluorine atom shown in the formulae.

In line with the above, the compounds in accordance with the invention encompass radiolabeled compounds and non-radiolabeled compounds. Radiolabeled compounds of the invention are compounds containing a radioactive constituent, such as a [¹⁸F]fluorine atom as the fluorine atom shown in formulae (Ia), (Ib), (Ic), (IIa), (IIb) and (IIc) or, if the compound comprises a chelate moiety, a chelate moiety comprising a chelated radioactive metal cation. Preferably, a radiolabeled compound in accordance with the invention is a compound wherein the fluorine atom shown in formulae (Ia), (Ib), (Ic), (IIa), (IIb) and (IIc) is [¹⁸F]fluorine. Non-radiolabeled compounds in accordance with the invention are compounds not containing a radioactive constituent, in particular compounds neither containing [¹⁸F]fluorine nor a chelated radioactive metal cation.

As another aspect, the invention provides a pharmaceutical composition, typically a radiopharmaceutical composition, comprising a compound in accordance with the invention, optionally in combination with a pharmaceutically acceptable carrier, excipient and/or diluent. A compound in accordance with the invention which is included in a radiopharmaceutical composition is generally a radiolabeled compound.

A composition comprising a non-radiolabeled compound in accordance with the invention, optionally in combination with a pharmaceutically acceptable carrier, excipient and/or diluent, can be provided e.g. as a precursor composition for a radiopharmaceutical composition. In a related aspect, the invention provides a kit comprising a non-radiolabeled compound in accordance with the invention which does not contain a radioactive component, optionally in combination with a pharmaceutically acceptable excipient, in combination with instructions for radiolabeling the compound.

In another aspect, the present invention provides a diagnostic composition comprising a compound in accordance with the invention, typically a radiolabeled compound in accordance with the invention, optionally in combination with a pharmaceutically acceptable carrier, excipient and/or diluent. In a related aspect, the invention provides a compound in accordance with the invention, typically a radiolabeled compound in accordance with the invention, or a radiopharmaceutical composition as discussed above, for use in a method of diagnosis in vivo of a disease or disorder. The method of diagnosis preferably involves nuclear diagnostic imaging, e.g. via Positron Emission Tomography (PET) or Single Photon Emission Computed Tomography (SPECT). A radiolabeled compound in accordance with the invention wherein the fluorine atom shown in formulae (Ia), (Ib), (Ic), (IIa), (IIb) and (IIc) is [¹⁸F]fluorine is a preferred radiolabeled compound suitable for use in PET.

For example, the method of diagnosis may comprise administering a radiolabeled compound in accordance with the invention to a subject and detecting the compound in the subject, or monitoring the distribution of the compound in the subject, thereby detecting or monitoring the disease or disorder to be diagnosed. The subject may be a human or an animal and is preferably human. Alternatively, a method of diagnosis may also comprise adding a radiolabeled compound in accordance with the invention to a sample, e.g. a physiological sample obtained from a subject in vitro or ex vivo, and detecting the compound in the sample.

The method of diagnosis referred to above aims at the identification of a disease or disorder of the human or animal body. An example for a disease or disorder is cancer. If the compound in accordance with the invention is a compound of formula (IIa), (IIb) or (IIc), it will be understood that the disease or disorder is typically one which is associated with the target structure of the targeting moiety comprised by the group R^{E}. For example, the presence or the increased presence of the target structure in the body of the subject may be indicative of the disease or disorder. In particular, target structures of interest, such as the ones discussed as examples above, may be overexpressed in a human disease, such as cancer.

In still a further aspect, the present invention provides a therapeutic composition comprising a compound in accordance with the invention, typically a radiolabeled compound in accordance with the invention, optionally in combination with a pharmaceutically acceptable carrier, excipient and/or diluent. In a related aspect, the invention provides a compound in accordance with the invention, typically a radiolabeled compound in accordance with the invention, or a radiopharmaceutical composition as discussed above, for use in a method of treatment of a disease or disorder. The method is preferably a method for the treatment of a disease or disorder via radioligand therapy. As an example for a radiolabeled compound in accordance with the invention useful for radioligand therapy, reference can be made to a compound comprising a chelate moiety wherein the chelated metal cation is a ¹⁷⁷Lu cation.

For example, the method of treatment may comprise administering a radiolabeled compound in accordance with the invention to a subject. The subject may be a human or an animal and is preferably human.

The method of treatment referred to above aims at the treatment of a disease or disorder of the human or animal body. An example for a disease or disorder is cancer. If the compound in accordance with the invention is a compound of formula (IIa), (IIb) or (IIc), it will be understood that the disease or disorder is typically one which is associated with the target structure of the targeting moiety comprised by the group R^{E}.

It will be understood that suitability for a therapeutic and a diagnostic application is not mutually exclusive. i.e. a compound in accordance with the invention may be suitable for both applications. For example, a compound comprising a chelated ¹⁷⁷Lu cation can be used both for therapeutic and diagnostic imaging applications. Moreover, compounds of the invention which comprise a chelating group together with the SiFA group are suitable as radiohybrid (rh) ligands. Such a rh ligand can be alternatively labeled with [¹⁸F]fluoride (e.g. for PET) or a radiometal (such as a ⁶⁸Ga cation for PET, or a ¹⁷⁷Lu cation for radiotherapy). When a rh ligand is labeled with [¹⁸F]fluoride, a cold (non-radioactive) metal cation can (but does not have to be) complexed by a chelator moiety elsewhere in the molecule, and when it is labeled with a corresponding radioactive metal cation, cold [¹⁹F]fluorine can be included. Therefore, the ¹⁸F-labeled peptide and the corresponding radiometal-labeled analog can possess the same chemical structure and thus identical in vitro and in vivo properties, thereby allowing the generation of structurally identical theranostic tracers with exactly the same in vivo properties of the diagnostic and therapeutic tracers (e.g. ¹⁸F/¹⁷⁷Lu analogs).

Thus, in line with this approach, the compounds in accordance with the invention include radiolabeled compounds wherein the SiFA group is labeled with ¹⁸F and a chelate moiety is present which contains a chelated non-radioactive cation (such as ^{nat}Lu or ^{nat}Ga), and radiolabeled compounds wherein a chelate moiety is present and contains a chelated radioactive cation (such as ¹⁷⁷Lu or ⁶⁸Ga) and the SiFA group is not labeled with ¹⁸F (thus carrying a ¹⁹F). Likewise, the invention provides such compounds in accordance with the invention for use in a hybrid method of diagnosis in vivo and therapy of a disease or disorder, wherein the method involves first the administration of a compound of the invention wherein the SiFA group is labeled with ¹⁸F and the chelating group contains a chelated non-radioactive cation (such as ^{nat}Lu or ^{nat}Ga), and subsequently of a compound wherein the chelating group contains a chelated radioactive cation and the SiFA group is not labeled with ¹⁸F.

In a related aspect, the compound in accordance with the invention can be used in an imaging method, which method may comprise administering the ligand compound to a subject and detecting the compound in the subject and monitoring the distribution of the compound in vivo at different time points after injection with the aim to calculate the dosimetry prior or during a therapeutic treatment. The subject may be a human or an animal and is preferably human. Such an imaging method may be relied on for the calculation of the dosimetry prior or during a therapeutic treatment of a disease or disorder of the human or animal body via radioligand therapy.

As noted above, compositions provided by the invention may comprise a pharmaceutically acceptable carrier, excipient and/or diluent. Examples of suitable pharmaceutical carriers, excipients and/or diluents are well known in the art and include phosphate buffered saline solutions, amino acid buffered solutions (with or without saline), water for injection, emulsions, such as oil/water emulsions, various types of wetting agents, sterile solutions etc. Such compositions can be formulated by well-known conventional methods.

In this specification, a number of documents including not only scientific journal articles but also patent applications and manufacturer's manuals are cited (cf, e.g., the list of references below in this respect). The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### EXAMPLES

Hereafter, the synthesis of SiFA groups in accordance with the present invention as well as the evaluation of their fundamental properties compared to the current gold-standard, p-(di-tert-butyl-(fluorosilyl)benzoic acid ((4-SiFA)benzoic acid, (4-SiFA)Bz), are illustrated using various model peptides. Additionally, the performance of the SiFA groups is exemplified by the use within compounds in accordance with the invention containing a variety of targeting vectors applied in the fields of radiopharmacy.

### 1. COMPARATIVE EVALUATION OF CHEMICAL PROPERTIES OF VARIOUS SIFA-MOIETIES BASED ON MODEL PEPTIDES (MPS)

Initially, each prosthetic group was coupled to three different model peptides (MPs, Table 1) to investigate the structural influence of the novel SiFA-building blocks in comparison to the gold-standard on labeling properties, stability and lipophilicity of the model peptides. As similar trends were observed for these three series, the modifiability of in vitro properties was then only investigated by an extension of the series a (X-Gly-Lys, see Table 2) offering differences in HSA-binding strengths within a region distinguishable via the high-performance affinity chromatography (HPAC) method.

TABLE 1
Structures, purities and yields of the investigated model peptides (MP) comprising of the dipeptides
a-c, and the various SiFA-groups, namely the gold-standard (4-SiFA)Bz (R₁), (3-SiFA)Bz (R₂), the newly
developed (5-SiFA)Ip (R₃) and modified amino acid conjugates of the latter (HO-L-Lys-(5-SiFA)Ip (R₄) and
(HO-L-Glu-(5-SiFA)Ip (R₅)).

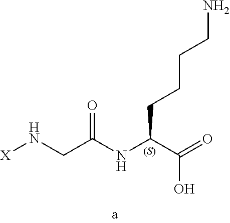

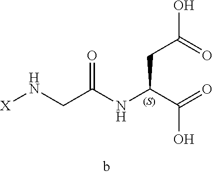

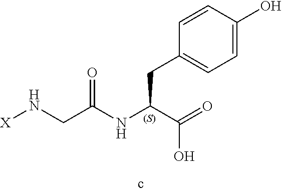

compound 1a-c: X = R₁

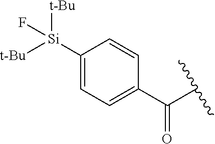 [SMILES: *C(=O)c1ccc([Si](F)(C(C)(C)C)C(C)(C)C)cc1]

R₁ = (4-SiFA)Bz
compound 2a-c: X = R₂

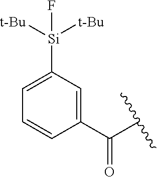 [SMILES: *C(=O)c1cccc([Si](F)(C(C)(C)C)C(C)(C)C)c1]

R₂ = (3-SiFA)Bz
compound 3a-c: X = R₃

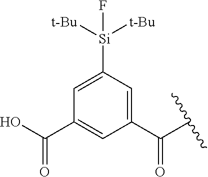 [SMILES: *C(=O)c1cc(C(=O)O)cc([Si](F)(C(C)(C)C)C(C)(C)C)c1]

R₃ = (5-SiFA)Ip
compound 4a-c: X = R₄

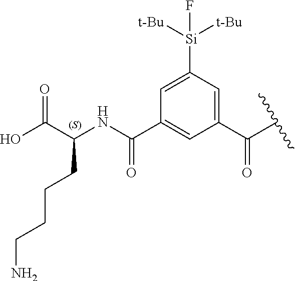

R₄ = HO-L-Lys-(5-SiFA)Ip
compound 5a-c: X = R₅

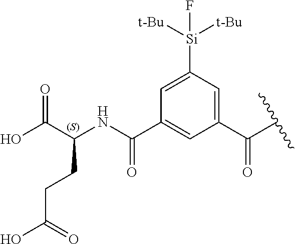

R₅ = HO-L-Glu-(5-SiFA)Ip

| Compound | Peptide Sequence | Purity (HPLC) [%] | Yield [%] |
|---|---|---|---|
| 1a** | (4-SiFA)Bz-Gly-L-Lys-OH | 97.5 | 77 |
| 1b** | (4-SiFA)Bz-Gly-L-Asp-OH | 98.6 | 86 |
| 1c** | (4-SiFA)Bz-Gly-L-Tyr-OH | 95.7 | 90 |
| 2a** | (3-SiFA)Bz-Gly-L-Lys-OH | 96.9 | 95 |
| 2b** | (3-SiFA)Bz-Gly-L-Asp-OH | 95.2 | 79 |
| 2c** | (3-SiFA)Bz-Gly-L-Tyr-OH | 95.0 | 77 |
| 3a | (5-SiFA)Ip-Gly-L-Lys-OH | 98.8 | 25 |
| 3b | (5-SiFA)Ip-Gly-L-Asp-OH | 98.3 | 38 |
| 3c | (5-SiFA)Ip-Gly-L-Tyr-OH | 95.7 | 18 |
| 4a | HO-L-Lys-(5-SiFA)Ip-Gly-L-Lys-OH | 99.3 | 16 |
| 4b | HO-L-Lys-(5-SiFA)Ip-Gly-L-Asp-OH | 98.1 | 21 |
| 4c | HO-L-Lys-(5-SiFA)Ip-Gly-L-Tyr-OH | 94.5 | 9 |
| 5a | HO-L-Glu-(5-SiFA)Ip-Gly-L-Lys-OH | 96.1 | 12 |
| 5b | HO-L-Glu-(5-SiFA)Ip-Gly-L-Asp-OH | 96.7 | 23 |
| 5c | HO-L-Glu-(5-SiFA)Ip-Gly-L-Tyr-OH | 95.3 | 15 |

**comparative data

TABLE 2
Structures, purities and yields of the investigated model peptides (MP) as extension of the Lys-series a.
3a*

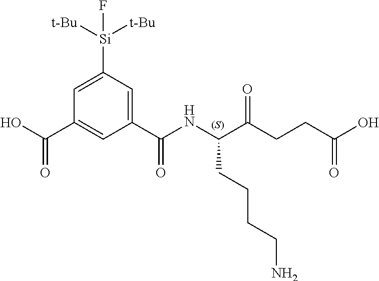

6a

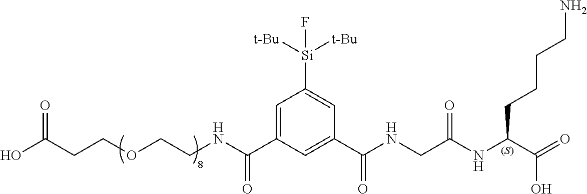

7a

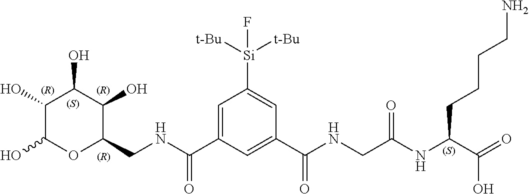

| | | Purity (HPLC) | Yield |
|---|---|---|---|
| Compound | Peptide Sequence | [%] | [%] |
| 3a* | (5-SiFA)Ip-L-Lys-Gly-OH | 99.0 | 26 |
| 6a | HOOC-PEG₈-(5-SiFA)Ip-Gly-L- | 98.3 | 25 |
| | Lys-OH | | |
| 7a | Gal6N-(5-SiFA)Ip-Gly-L-Lys-OH | 99.2 | 17 |

### 1.1 ¹⁸F-Labeling of Model Peptides (MPs)

The efficiency of the isotopic exchange for ¹⁸F-labeling was evaluated by the determination of the radiochemical conversion (RCC, n≥3) analyzing a sample of the reaction mixture by thin-layer chromatography (TLC) and the determination of the decay-corrected radiochemical yield (RCY, n≥3) after purification via cartridge (see Figure).

All derivatives showed good RCCs and RCYs within a range of approximately 60-80% with molar activities of 3.1±1.8 GBq/μmol. A trend is observable with quite similar values for (4-SiFA)Bz- and (5-SiFA)Ip-derivatives, while both values tend to be slightly decreased for the (3-SiFA)Bz- and slightly increased for X-(5-SiFA)Ip-derivatives, with X=-L-Lys-OH, -L-Glu-OH, -Gal6N or -PEG₈-OH.

Trend for RCC and RCY of ¹⁸F-labeling reactions with model peptides (MP):

$$( 3 - SiFA ) Bz - MPs < ( 4 - SiFA ) Bz - MPs ∼ ( 5 - SiFA ) / p - MPs < X - ( 5 - SiFA ) / p - MPs$$

1.2 Relative Radiochemical Conversion (rRCC)

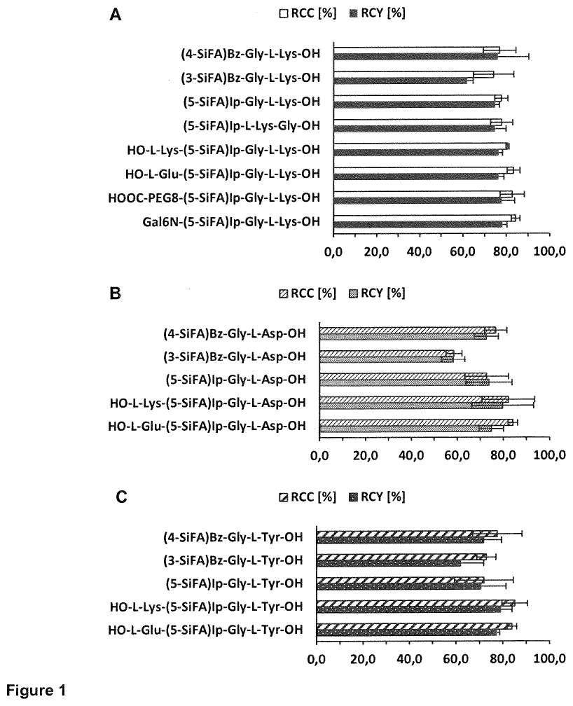

For a more precise comparison of the relative reactivity of the various SiFA-model peptides in the isotopic exchange reaction, competitive ¹⁸F-labelings were carried out. Herein, the corresponding (4-SiFA)Bz-MP was chosen as internal reference. Equal amounts of the test- and reference-compound were added as one solution to an anhydrous [¹⁸F]F-solution. After 5 min at rt, a sample was analyzed via radio-RP-HPLC. The relative radiochemical conversion (rRCC) was calculated by peak integration and is expressed as the ratio of test-to-reference-compound [%]. It was concluded that the rRCC allows for a more precise comparison of reactivities as opposed to the standard RCC (see FIGS. 1 A-C) determined in separately conducted experiments.

FIGS. 1 A-C show radiochemical conversions (RCC, n≥3) and radiochemical yields (RCY, n≥3) for ¹⁸F-labeling reactions (5 min, rt, molar activity=3.1±1.8 GBq/μmol) of the model peptides divided into Lys-, Asp and Tyr-series and depicted in A, B and C, respectively.

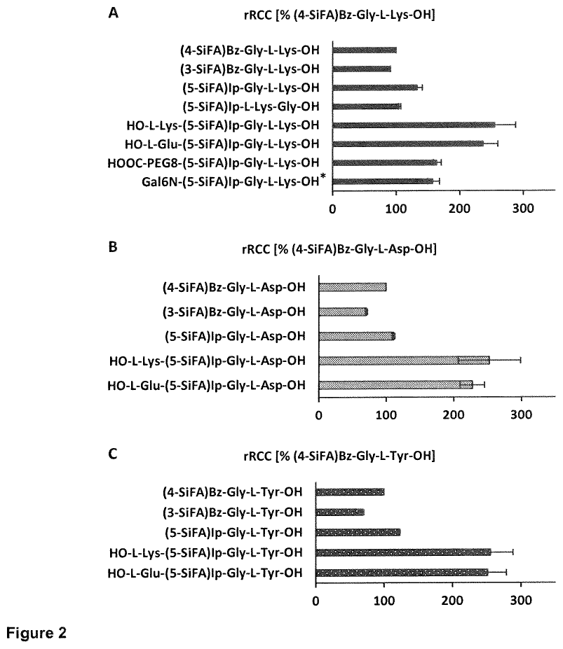

Therefore, the order of ranking corresponding to the RCCs ((3-SiFA)Bz-MPs<(4-SiFA)Bz-MPs˜(5-SiFA)Ip-MPs<X-(5-SiFA)Ip-MPs) can be better differentiated by the comparison of the rRCCs, as depicted in FIGS. 2 A-C, and results in the ranking below:

$$( 3 - SiFA ) Bz - MPs < ( 4 - SiFA ) Bz - MPs < ( 5 - SiFA ) / p - MPs < X - ( 5 - SiFA ) / p - MPs$$

$$( ca . 75 \% < 100 \% < 125 \% < 150 - 250 \% )$$

FIGS. 2 A-C shows the relative reactivities represented by rRCCs standardized to the conversion of the corresponding (4-SiFA)Bz-MPs individually determined (n=3) by the labeling of an equimolar precursor mixture of the test compound and its (4-SiFA)Bz-counterpart depicted at the top of each series A, B and C dividing data into Lys-, Asp and Tyr-series, respectively. *Quality controls via HPLC show the formation of two unidentified side products (approximately 5-10%) for the Gal6N-derivative which were added to the test compound integral and are estimated to occur by virtue of keto- and enol tautomerism at the sugar moiety.

Thus, in this context it can be assessed that the novel (5-SiFA)Ip- and X-(5-SiFA)Ip-derivatives exhibit better labeling properties, especially increased reactivities resulting in higher RCYs.

1.3 Determination of the n-Octanol-Water Partition Coefficient (log D_{7.4})

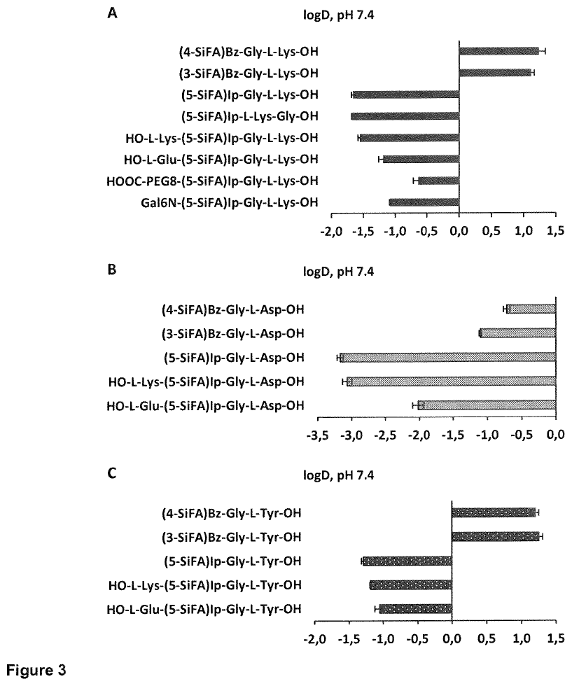

In the development process of alternative SiFA-analogs, the influence of the substitution pattern at the aromatic moiety on lipophilicity is of particular importance. As the so far used 4-SiFA-Bz-moiety exhibits a strong lipophilic character which is also a dominating factor for the property of a SiFA-ligand conjugate, it was of major interest, if the newly developed SiFA-moieties would lead to a superior hydrophilicity represented by a more negative log D_{7.4}-value. Assuming to observe a better differentiation in properties of the various prosthetic groups, investigations were conducted on small model peptides. Corresponding log D_{7.4}-values (n≥8) are depicted in FIG. 3.

FIG. 3 A-C shows the n-octanol-water partition coefficient (log D_{7.4}, n 8) of ¹⁸F-labeled model peptides divided into Lys-, Asp and Tyr-series and depicted in A, B and C, respectively.

Results in FIG. 3 A-C indicate only a moderate shift in lipophilicity comparing (4-SiFA)Bz- with (3-SiFA)Bz-derivatives. In contrast, the negative charge in close proximity to the aromatic moiety introduced by the additional carboxylic acid itself within the (5-Sifa)Ip-motif as well as the X-(5-Sifa)Ip-moiety, with X=-L-Lys-OH, -L-Glu-OH, -Gal6N or -PEG₈-OH as a variable hydrophilic modifier easily conjugated to this additional carboxylic acid via peptide chemistry, lead to a drastically improved hydrophilicity.

### 1.4 Determination of Human Serum Albumin (HSA) Binding by High Performance Affinity Chromatography (HPAC)

Investigations about the binding strength of the SiFA-MPs to human serum albumin (HSA) were conducted using high performance affinity chromatography (HPAC). The corresponding gold-standards ((4-SiFA)Bz-MPs) display—as expected from published as well as internal data—a quite strong HSA binding. This is associated with a comparably slow and unfavored blood clearance in vivo.

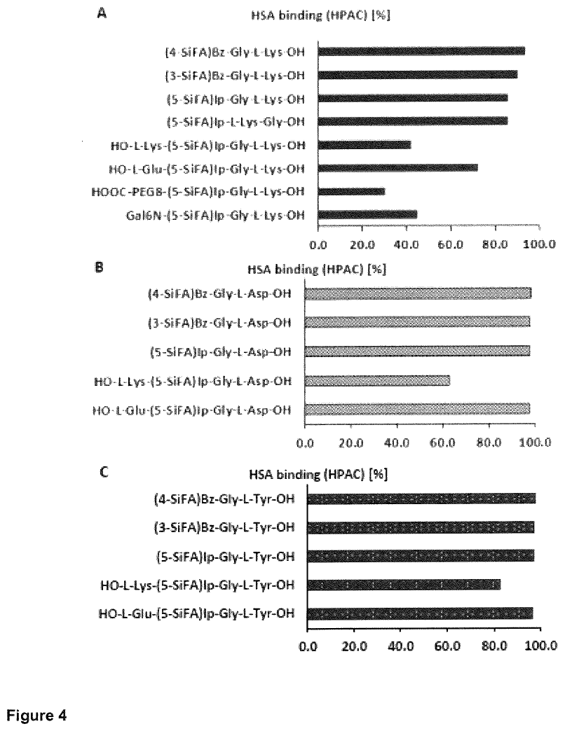

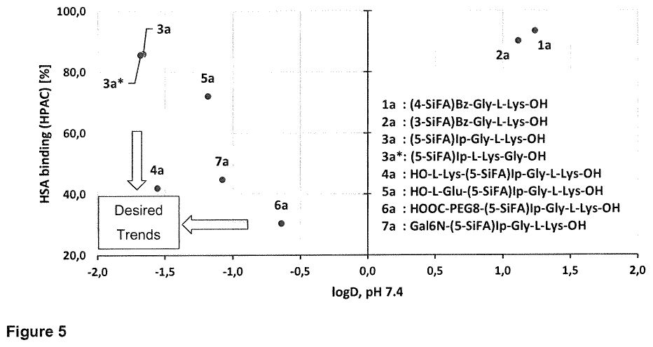

As it can be seen in FIG. 4 A-C, only minor differences in HSA binding were observed comparing (3-SiFA)Bz- and (5-SiFA)Ip-derivatives with (4-SiFA)Bz-derivatives. However, the HSA binding can be drastically decreased by conjugation of further modifiers to the (5-SiFA)Ip-moiety, illustrated in FIG. 4A and FIG. 5. In the latter figure, it is clearly depicted that especially the HO-L-Lys- (4a), HOOC-PEG₈- (6a) and Gal6N (7a)-modifier promise to be a valuable tool in further optimizing two key parameters crucial for desired fast pharmacokinetics, namely a decreased HSA binding and an improved hydrophilicity.

FIG. 4 A-C shows the HSA binding of the SiFA-MPs according to HPAC divided into Lys-, Asp and Tyr-series and depicted in A, B and C, respectively.

FIG. 5 shows a comparison of log D_{7.4} values vs. HSA binding for the Lys-Series a.

Thus, it can be summarized that the X-(5-SiFA)Ip-moiety can serve as an extremely useful platform not only for ¹⁸F-labeling but also as an HSA binding moiety with an HSA binding easily modulated over a broad range of binding strength by conjugating suitable modifiers of choice to the additional carboxylic acid of the newly developed (5-SiFA)Ip-moiety to adjust pharmacokinetic properties of a ligand exactly to the requirements in vivo.

### 1.5 Determination of the Chemical Stability of the [¹⁸F]F-Si Bond Under Various Conditions

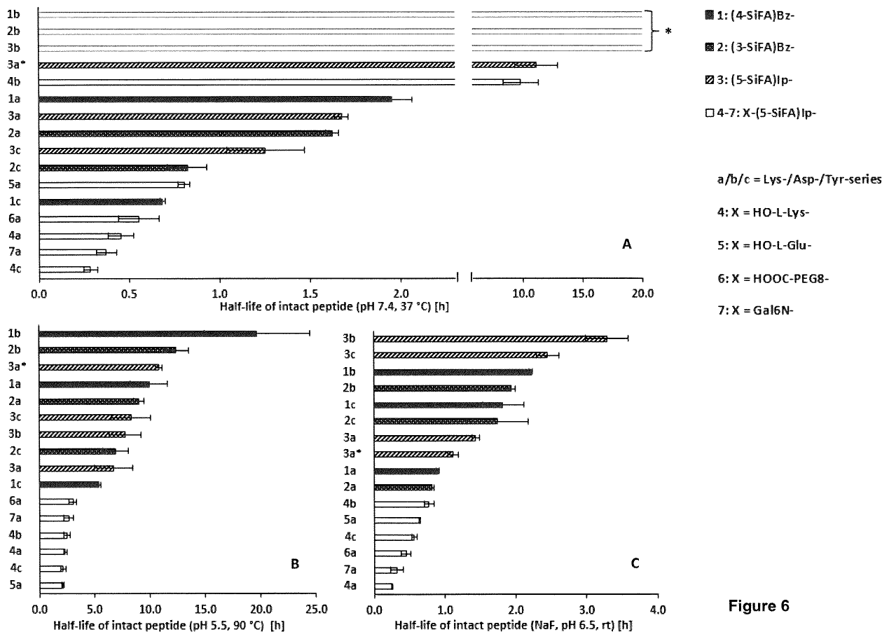

To determine the chemical stability of the [¹⁸F]F-Si bond within the various MPs, aliquots of the ¹⁸F-labeled MPs were incubated under three different conditions to simulate the in vivo stability in blood (pH 7.4, 37° C.), the stability under conditions of the ¹⁷⁷Lu-labeling protocol for SiFA-radiohybrids used by our group (pH 5.5, 90° C.) as well as the reactivity for the reversed isotopic exchange in aqueous media (1 mM NaF-solution). For quantification of the ¹⁸F-defluorination kinetics, samples were taken over a period of 2 h (t=0, 30, 60, 90 and 120 min) and the proportion of intact, ¹⁸F-labeled peptide was monitored by radio-TLC. The corresponding calculated half-lives (n=3) of the intact ¹⁸F-labeled MPs under the three different conditions are depicted in FIG. 6 A-C.

FIG. 6 shows the half-lives (n=3) of the intact ¹⁸F-labeled MPs divided into subsets A: pH 7.4 at 37° C. (simulated blood conditions), B: pH 5.5 at 90° C. (¹⁷⁷Lu-labeling protocol for SiFA-radiohybrids, and C: NaF (1 mM in H₂O, pH 6.5 at rt (reversed isotopic exchange); * indicates that half-lives are not given because of unprecise regression (R2<<90%) with stabilities of >95% intact MP after 2 h.

Noteworthy, previous in vitro- and in vivo-experiences validated the stability of (4-SiFA)Bz-derivatives as sufficient. As reflected by the data shown below, the peptide sequence (Asp- vs. Tyr- and Lys-MPs) and even the positioning of same amino acids ((5-SiFA)Ip-Gly-L-Lys-OH vs. (5-SiFA)Ip-L-Lys-Gly-OH) seem to have a drastic influence on the observed half-life of intact MP. Furthermore, with respect to the standard deviation, corresponding (4-SiFA)-Bz-, (3-SiFA)-Bz-, and (5-SiFA)Ip-MPs within one MP-series mostly display similar stabilities while X-(5-SiFA)Ip-MPs (depicted in white bars) are determined to be less stable. These findings are in well accordance with the ¹⁸F-labeling results indicating that more reactive derivatives with higher (r)RCCs and RCYs also show faster ¹⁸F-defluorination in the stability assay. Nevertheless, although X-(5-SiFA)Ip-MPs showed to be less stable than corresponding (4-SiFA)Bz-MPs, it can be concluded that the stabilities of the novel compounds seem to be in a range feasible for in vivo applications.

### 2. COMPARATIVE EVALUATION OF CHEMICAL PROPERTIES OF VARIOUS SIFA-MOIETIES EXEMPLIFIED BY VECTOR-BASED LIGANDS

In the following, exemplifying data of several vector-based ligands are shown to demonstrate advantages of the novel (5-SiFA)Ip-based building block.

### 2.1. Gastrin-Releasing Peptide Receptor (GRPR) Ligands

TABLE 3
Structures, HSA binding, GRPR affinity (IC₅₀) and n-octanol-water partition coefficient (logD_{7.4}, n = 6, ¹⁷⁷Lu-
labeled compound) of radiohybrid GRPR ligands sharing the peptide sequence Lu-DOTAGA-D-Dap(Rₓ)-EDA-DOTA-[α-
Me-Trp⁸]MJ9 comparing the gold-standard (4-SiFA)Bz- with the (3-SiFA)Bz- and the novel (5-SiFA)Ip-moiety.

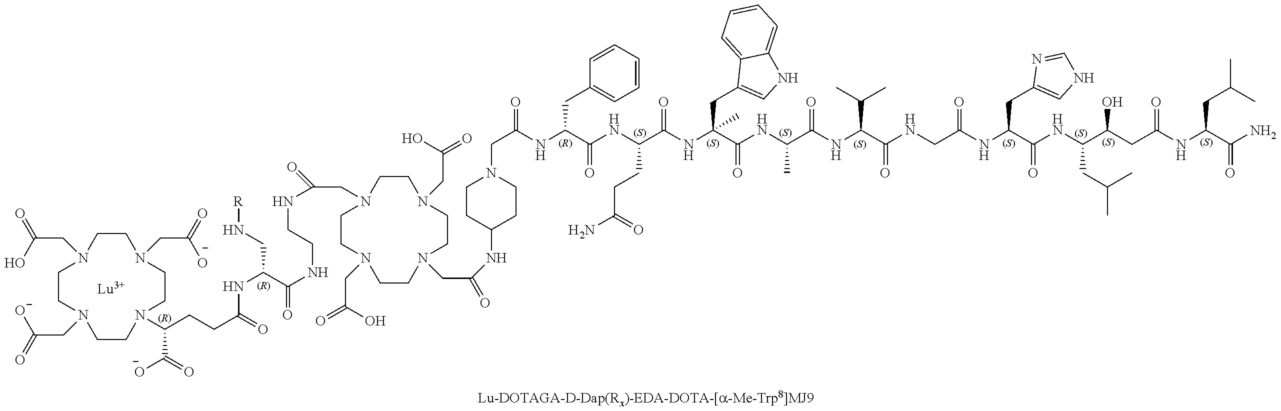

| | | |
|---|---|---|
| R: R₁ = (4-SiFA)Bz- | R: R₂ = (3-SiFA)Bz- | R: R₃ = (5-SiFA)Ip- |
| 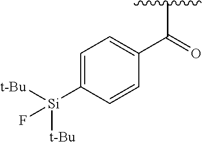 [SMILES: *C(=O)c1ccc([Si](F)(C(C)(C)C)C(C)(C)C)cc1] | 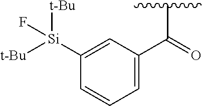 [SMILES: *C(=O)c1cccc([Si](F)(C(C)(C)C)C(C)(C)C)c1] | 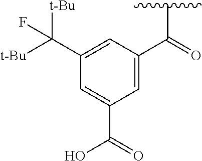 [SMILES: *C(=O)c1cc(C(=O)O)cc(C(F)(C(C)(C)C)C(C)(C)C)c1] |

| | | HSA binding (HPAC) | | |
|---|---|---|---|---|
| Entry | Modification Rₓ | [%] | IC₅₀ [nM] (n = 3) | logD_{7.4} (n = 6) |
| 1** | R₁: (4-SiFA)Bz- | 96.6 | 14.4 ± 1.9 | −1.03 ± 0.08 |
| 2** | R₂: (3-FiFA)Bz- | 93.3 | 14.9 ± 1.4 | −1.15 ± 0.10 |
| 3 | R₃: (5-SiFA)Ip- | 83.2 | 21.0 ± 2.9 | −1.87 ± 0.05 |

**comparative data

As depicted in Table 3, the change in modifications from the (4-SiFA)Bz- to (3-SiFA)Bz-compound leads to only a moderate improvement of the HSA binding and a negligible change in hydrophilicity. Indeed, the (5-SiFA)Ip-derivative shows a desirable, significant reduction in HSA binding as well as a significant increase in hydrophilicity.

Furthermore, the introduction of an additional charged group within the (5-SiFA)Ip-moiety was expected to potentially have an influence on the receptor affinity. Whether this influence positively or negatively affects the affinity needs to be investigated individually for each target and might be adapted in a subsequent development step by structure-activity relationships. Therefore, a loss in affinity by the initial use of the novel (5-SiFA)Ip-moiety in a single compound does not necessarily constitute a permanent limitation for a whole group of ligands. However, the results indicate a promising step using the (5-SiFA)Ip- or perhaps a further modifiable X-(5-SiFA)Ip-moiety instead of the (4-SiFA)Bz-moiety to compensate efficiently in structural proximity to the t-Bu₂FSi-Ar group for the high lipophilicity and strong HSA binding.

### 2.2. C-X-C Chemokine Receptor Type 4 (CXCR4) Ligands

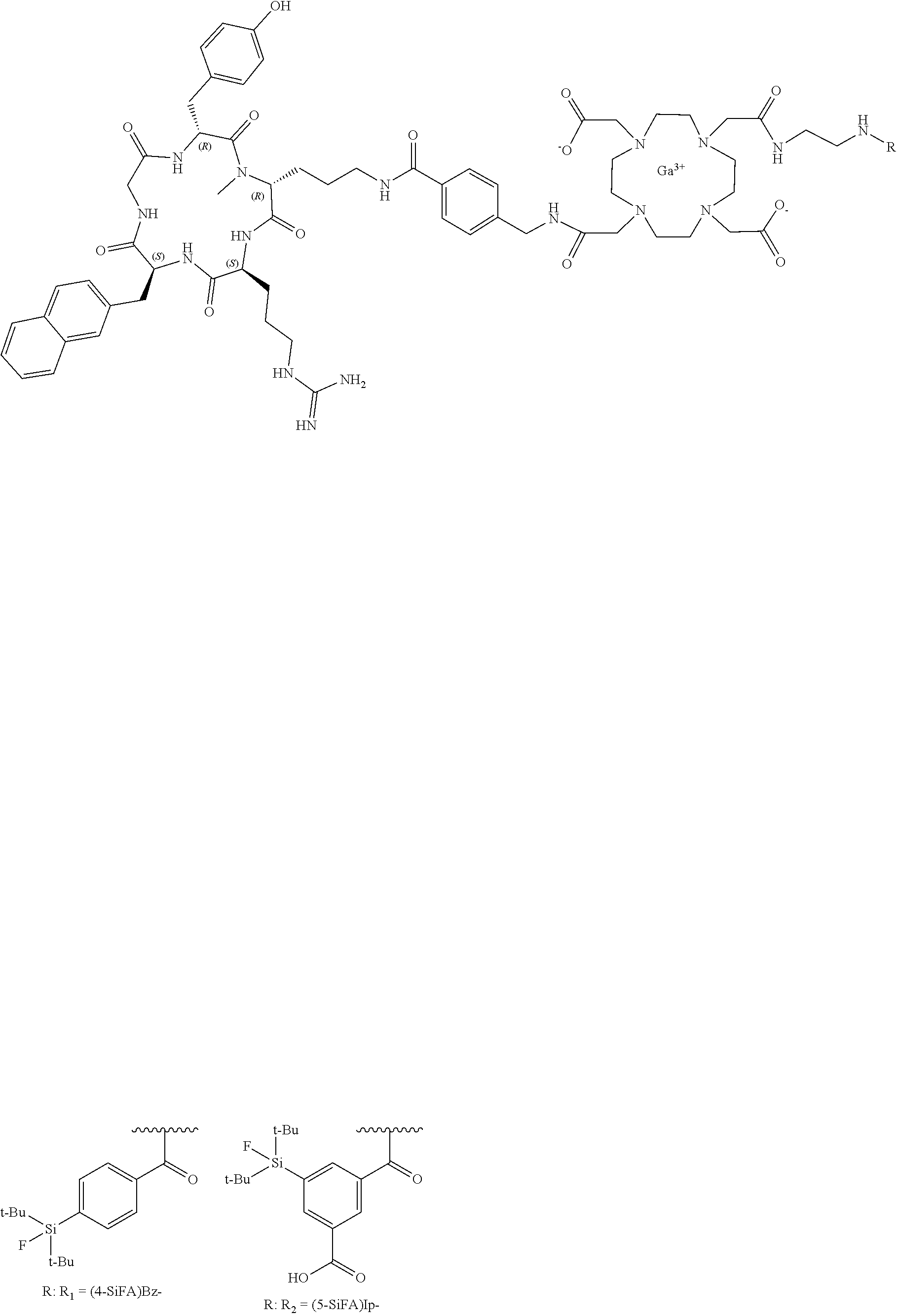

Structures of radiohybrid CXCR4 ligands sharing Pentixafor as lead structure modified with the gold-standard (4-SiFA)Bz- or the novel (5-SiFA)Ip-moiety attached via an EDA-linker at the chelator.

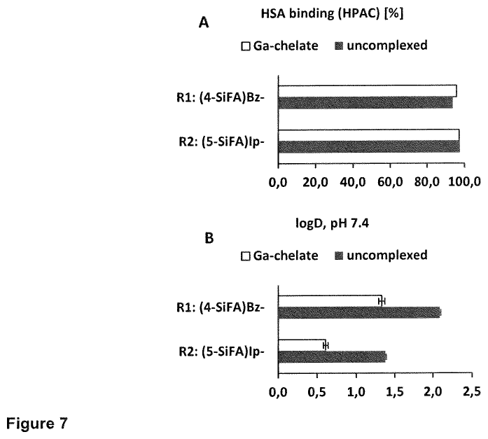

FIG. 7 A, B shows the HSA binding and n-octanol-water partition coefficient (log D_{7.4}, n=8, ¹⁸F-labeled compound) of radiohybrid CXCR4 ligands sharing Pentixafor as lead structure modified with the gold-standard (4-SiFA)Bz- or the novel (5-SiFA)Ip-moiety attached via an EDA-linker at the chelator.

The above depicted derivatives of the well-known CXCR4 ligand Pentixafor display the same expected trend of a significantly reduced lipophilicity attaching the (5-SiFA)Ip-moiety instead of the standard (4-SiFA)Bz-moiety. This effect is even obvious in the presence of—and not dominated by—the lipophilic amino acids installed in the pharmacophore, namely NaI and Tyr, as well as the 4-Abz linker-moiety. The HSA binding is seemingly not influenced by the change of the prosthetic group probably indicating a dominating interaction of the aromatic amino acids of the pharmacophore towards HSA.

### 2.3. Somatostatin Receptor (SSTR) Ligands

Similar to the above shown CXCR4 ligands, derived from Pentixafor as shown in 2.2 above, the cyclic octapeptide Octreotate (TATE) constitutes a rather lipophilic pharmacophore. Thus, consisting of a pharmacophore and a prosthetic group both separately already being lipophilic, it is not surprising to likewise observe a high lipophilicity for the (4-SiFA)Bz-conjugates (Table 4, Entry 1, 2).

As opposed to that, both (5-SiFA)Ip-derivatives display a gain in hydrophilicity of 1.2-1.5 logarithmic units (Table 4, Entry 3, 4) compared to the corresponding (4-SiFA)Bz-conjugates. Interestingly, the installation of a carboxylic acid directly at the SiFA-moiety (Table 4, Entry 4) leads to a significantly superior increase in hydrophilicity compared to the (4-SiFA)Bz-derivative with the carboxylic acid shifted to the linker (Table 4, Entry 1). This finding clearly demonstrates the superior efficiency and utility of the novel (5-SiFA)Ip-moiety. Furthermore, additional modifications can be combined with the (5-SiFA)-moiety to achieve an even higher boost in hydrophilicity (Table 4, Entry 3).

Analogously to the CXCR4 ligands mentioned above, the HSA binding is barely influenced by the investigated modifications, as the interactions with HSA seem to be dominated by the strongly binding aromatic amino acids within the cyclic pharmacophore.

TABLE 4
Structures, HSA binding, SSTR affinity (IC₅₀) and n-octanol-water partition coefficient (logD_{7.4}, n = 6, ¹⁸F-
labeled compound) of radiohybrid SSTR ligands sharing the ^{nat}Ga-DOTATATE as pharmacophore comparatively
modified with the gold-standard (4-SiFA)Bz- and the novel (5-SiFA)Ip-moiety.

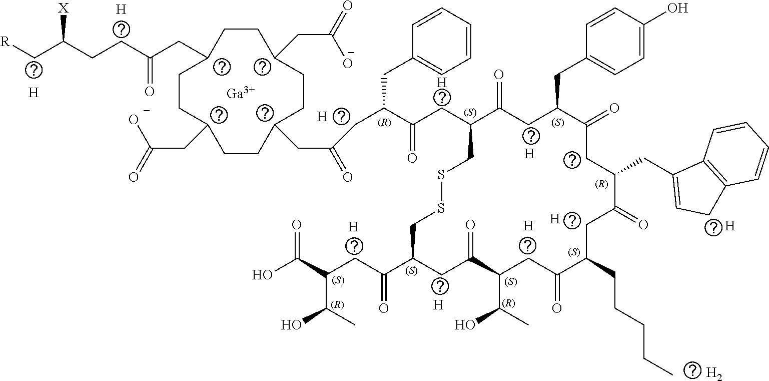

X: X₁ = −COOH
X: X₂ = −H
R: R₁ = (4-SiFA)Bz-

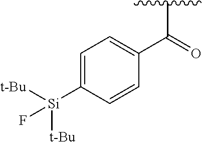 [SMILES: *C(=O)c1ccc([Si](F)(C(C)(C)C)C(C)(C)C)cc1]

R: R₂ = (5-SiFA)Ip-

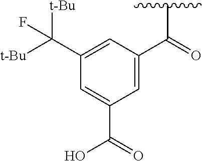 [SMILES: *C(=O)c1cc(C(=O)O)cc(C(F)(C(C)(C)C)C(C)(C)C)c1]

| | | HSA binding (HPAC) | | |
|---|---|---|---|---|
| Entry | Modifications R, X | [%] | IC₅₀ [nM] (n = 3) | logD_{7.4} (n = 6) |
| 1** | R₁: (4-SiFA)Bz-, X₁: —COOH | 98.6 | 4.6 ± 0.2 | −0.14 ± 0.05 |
| 2** | R₂: (4-SiFA)Bz-, X₂: —H | 96.4 | 6.7 ± 1.8 | 0.61 ± 0.03 |
| 3 | R₁: (5-SiFA)Ip-, X₁: —COOH | 96.2 | 4.3 ± 1.3 | −1.72 ± 0.03 |
| 4 | R₂: (5-SiFA)Ip-, X₂: —H | 97.4 | 4.6 ± 1.5 | −0.67 ± 0.03 |

**comparative data

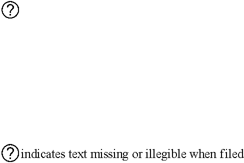

### 2.4. Cholecystokinin B Receptor (CCK-2R) Ligands

An additional example is constituted by ligands addressing the CCK-2 receptor. Again, likewise to the CXCR4 and SSTR ligands the pharmacophore contains several lipophilic aromatic amino acids (NaI, Trp and Tyr). Therefore, a high HSA binding as well as a high lipophilicity is expected. The conjugation of the hydrophilic DOTA chelator for Lu-complexation resulting in the reference compound Lu-DOTA-MGS5 compensates this lipophilicity to a log D value of −2.25 (8 D). Not surprisingly, the introduction of the (4-SiFA)Bz- or (3-SiFA)Bz-moiety via the trifunctional linker D-Dap leads to a drastic deterioration of the hydrophilicity with an increase of the log D values of about 2-2.5. In contrast, this shift can be efficiently minimized by the use of the novel (5-SiFA)Ip-moiety instead. In conclusion, another example reinforces the importance of the second carboxylic acid attached directly at the aromatic unit within the (5-SiFA)Ip-moiety to minimize the lipophilicity added by the introduction of a SiFA-based prosthetic group.

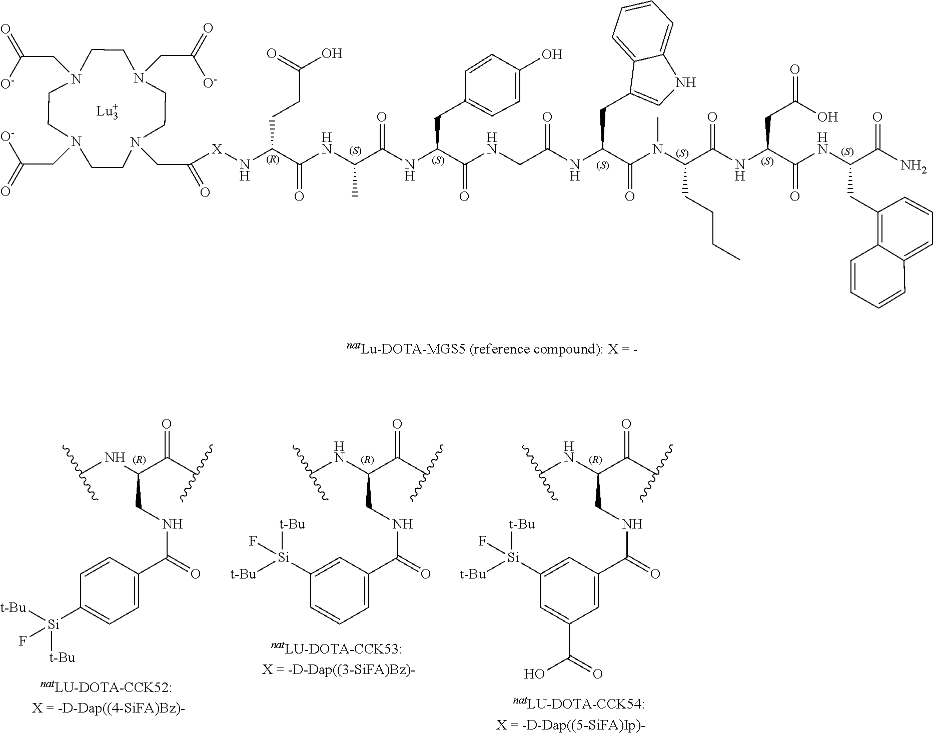

Structures of CCK-2R ligands, namely the reference compound ^{nat}Lu-DOTA-MGS5 and radiohybrid derivatives thereof sharing the same pharmacophore comparatively modified with the gold-standard (4-SiFA)Bz-, (3-SiFA)Bz- and the novel (5-SiFA)Ip-moiety.

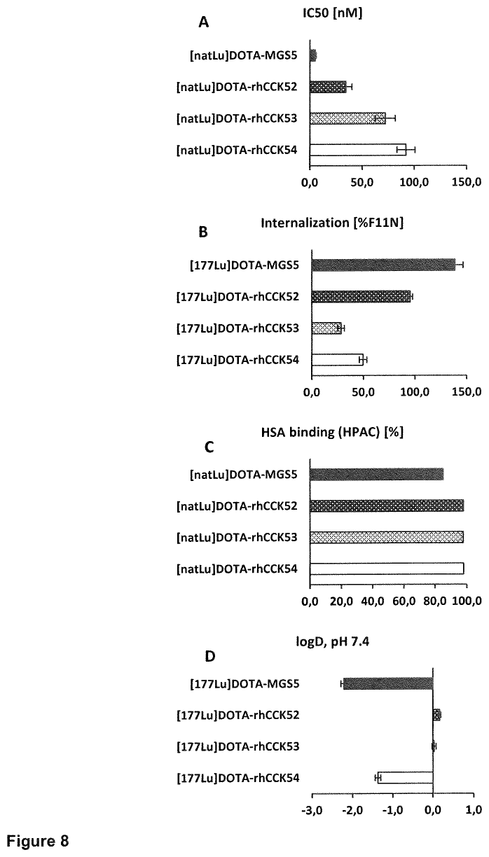

FIG. 8 A-D shows the CCK-2R affinity (IC₅₀, n=3), internalization (n=3) in % of the co-evaluated reference F11N, HSA binding (HPAC) and n-octanol-water partition coefficient (log D_{7.4}, n≥6, ¹⁷⁷Lu-labeled compound) of CCK-2R ligands, namely the reference compound ^{nat}Lu-DOTA-MGS5 and radiohybrid derivatives thereof sharing the same pharmacophore comparatively modified with the gold-standard (4-SiFA)Bz-, (3-SiFA)Bz- and the novel (5-SiFA)Ip-moiety attached via the trifunctional linker -D-Dap-.

### 2.5. Prostate-Specific Membrane Antigen (PSMA) Ligands

Further investigations were conducted using ligands addressing the prostate-specific membrane antigen (PSMA). Table 5 summarizes data of simplistic ligands comprised of the EuK-binding motif attached to the varied SiFA-moieties, namely (4-SiFA)Bz- (first synthesized by Wirtz (1), but herein independently re-evaluated), (3-SiFA)Bz- and (5-SiFA)Ip-. The observed values for HSA binding are nearly equal.

The determined IC₅₀-values state a positive example for a structural-activity relationship where the shift of the silyl group at the aromatic moiety is clearly beneficial for a superior affinity. The simple shift of the silyl group from para- (PSMA-1) to meta-position (PSMA-2) causes a more than 20-fold increase in affinity which can be further improved by a factor of two attaching a carboxylic acid (PSMA-3).

Additionally, with this dataset a further example was found clarifying that the additional carboxylic acid directly attached to the SiFA-moiety drastically boosts the hydrophilicity as a difference of about one logarithmic unit is observed comparing Entry 1 and 2 with Entry 3.

TABLE 5
Structures, HSA binding, PSMA affinity (IC₅₀) and n-octanol-water partition coefficient (logD_{7.4}, n ≥ 6) of small
molecule, EuK-based PSMA ligands comparing the gold-standard (4-SiFA)Bz- with the (3-SiFA)Bz- and the novel (5-
SiFA)Ip-moety.

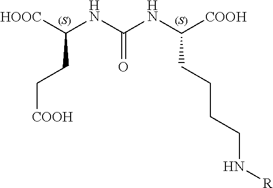

PSMA-1
R: R₁ = (4-SiFA)Bz-

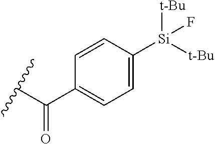 [SMILES: *C(=O)c1ccc([Si](F)(C(C)(C)C)C(C)(C)C)cc1]

PSMA-2
R: R₂ = (3-SiFA)Bz-

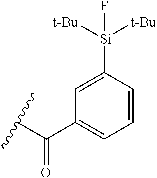 [SMILES: *C(=O)c1cccc([Si](F)(C(C)(C)C)C(C)(C)C)c1]

PSMA-3
R: R₃ = (5-SiFA)Ip-

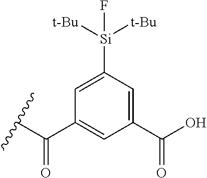 [SMILES: *C(=O)c1cc(C(=O)O)cc([Si](F)(C(C)(C)C)C(C)(C)C)c1]

| | | HSA binding (HPAC) | | |
|---|---|---|---|---|
| Entry | Modification R | [%] | IC₅₀ [nM] (n = 3) | logD_{7.4} (n = 6) |
| 1** | R₁: (4-SiFA)Bz- | 98.7 | 327 ± 73 | −1.95 ± 0.09 |
| 2 | R₂: (3-SiFA)Bz- | 98.2 | 13.7 ± 2.0 | −2.28 ± 0.06 |
| 3 | R₃: (5-SiFA)Ip- | 98.7 | 6.5 ± 1.6 | −3.12 ± 0.01 |

**comparative data

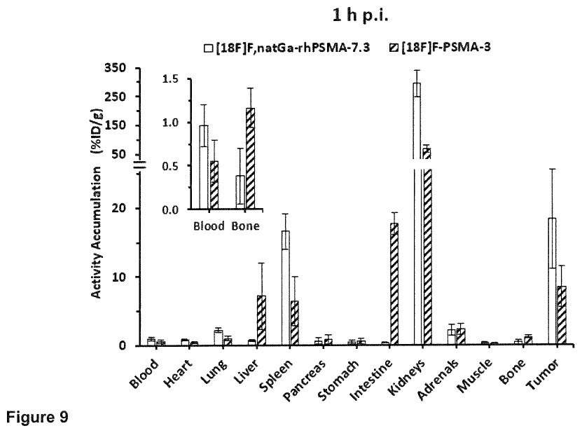

In vivo data were determined for [¹⁸F]F-PSMA-3 and compared with previously published data of [¹⁸F]F, ^{nat}Ga-rhPSMA-7.3 (2) (FIG. 9), not only validating the effect on pharmacokinetics, but especially indicating the stability of the respective [¹⁸F]F-Si-bond by comparison of the bone uptake.

FIG. 9 shows the biodistribution of the compounds [¹⁸F]F, ^{nat}Ga-rhPSMA-7.3 (n=4) and [¹⁸F]F-PSMA-3 (n=5) at 1 h p.i. in male tumor-bearing CB17-SCID mice. Data expressed as percentage of the injected dose per gram (% ID/g), mean±standard deviation. Data of [18F]F, ^{nat}Ga-rhPSMA-7.3 were previously published by our group. (2)

As depicted in FIG. 9, already 1 h p.i. [¹⁸F]F-PSMA-3 shows a faster blood clearance with a blood value of 0.55% ID/g compared to 0.96% ID/g for [¹⁸F]F, ^{nat}Ga-rhPSMA-7.3.

Obviously, the low kidney value (67.1±13.9% ID/g) of [¹⁸F]F-PSMA-3 is not directly comparable as this derivative seems to suffer from insufficient hydrophilicity in the absence of the hydrophilic DOTAGA chelate resulting in hepatobiliary excretion as indicated by increased accumulation in liver (7.2±4.8% ID/g) and intestine(17.7±1.6% ID/g).

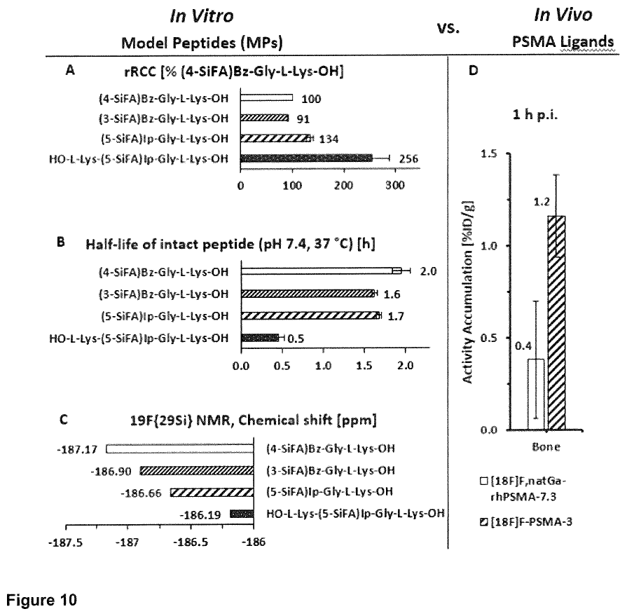

Although pharmacokinetics of these derivatives are clearly not identical resulting in unequal plasma half-lives and thus, deviating incubation conditions faced by the [¹⁸F]F-Si-bond within the derivatives, the values of bone uptake, assumed to be predominantly caused by cleaved [¹⁸F]Fluoride, reflect the trend in reactivity also found for model peptides in aforementioned results of in vitro studies (FIG. 10)

FIG. 10 shows the comparison of in vitro vs. in vivo data regarding reactivity and stability of the [¹⁸F]F—Si-bond as part of the different SiFA-moieties [(4-SiFA)Bz-, (3-SiFA)Bz-, terminal (5-SiFA)Ip- and bridging -(5-SiFA)Ip-]: A: Reactivity of model peptides (MPs) from the Lys-series under anhydrous [¹⁸F]F-labeling conditions expressed as relative radiochemical conversion (rRCC); B: Half-life of intact [¹⁸F]F-labeled MPs from the Lys-Series incubated in an aqueous K₂CO₃-solution (pH 7.4, 37° C.; simulated blood conditions); C: Chemical Shifts [ppm] of ¹⁹F{²⁹Si}NMR spectra of the Lys-series; D: Activity accumulation (% ID/g) in CB17-SCID mice (n≥4) 1 h p.i. of the ligands [¹⁸F]F, ^{nat}Ga-rhPSMA-7.3 and [¹⁸F]F-PSMA-3. Data of [¹⁸F]F,^{nat}Ga-rhPSMA-7.3 were previously published by our group. (2)

In vitro results of the Lys-MP series, depicted in FIG. 10A, display a trend in reactivity with a relative radiochemical conversion (rRCC) of the (3-SiFA)Bz-MP similar to the normalized value of the (4-SiFA)Bz-MP, a slightly higher rRCC for the terminal (5-SiFA)Ip-derivative by a factor of 1.3 and a clearly higher rRCC for the bridging (5-SiFA)Ip-derivative by a factor of about 2.6.

The increased reactivity was also found to be accompanied by a lower stability (FIG. 10B) with a half-life of the [¹⁸F]F-Si-bond within the MPs reduced by 15% attaching the (5-SiFA)Ip-moiety terminally and divided by a factor of 4.0 attaching a (5-SiFA)Ip-moiety in a bridging construct instead of the classical (4-SiFA)Bz-moiety, respectively.

Furthermore, there seems to be a correlation of enhanced reactivity and decreased stability with a less negative ¹⁹F NMR-shift (FIG. 10C), representing a lower electron density at the fluor atom.

Although the in vitro data (FIG. 10A-C) were not determined for the PSMA ligands, nevertheless, the found data of the MPs seem to have a predictive value for the trend of in vivo stability of the [¹⁸F]F-Si-bonds within the PSMA ligands indicated by activity accumulation in the bones, mainly due to dissociated [¹⁸F]Fluoride (FIG. 10D). Using the (5-SiFA)Ip-moiety terminally in [¹⁸F]F-PSMA-3, leaded to a bone uptake of 1.16±0.22% ID/g. Comparatively, the (4-SiFA)Bz-moiety utilized in [18F]F,^{nat}Ga-rhPSMA-7.3 proofed a higher stability with a bone uptake of 0.38±0.32% ID/g. (2)

Nevertheless, clinical translation of a (5-SiFA)Ip-moiety seems feasible despite of an increased [¹⁸F]Fluoride release in mice comparing the found bone uptake with results published about [¹⁸F]F-SiFAlin-TATE (Femur: 1.9±0.6% ID/g at 60 min p.i. (estimated by figure); 1.31±0.31% ID/g at 90 min p.i.). (3) First clinical results about [¹⁸F]F-SiFAlin-TATE in direct comparison with [⁶⁸Ga]Ga-DOTA-TOC in 13 patients lead to the conclusion of favorable ‘characteristics’. (4) In this study, a slightly higher bone uptake was found for [¹⁸F]F-SiFAlin-TATE compared to the non-fluorinated [¹⁸Ga]Ga-DOTA-TOC (SUVₘₐₓ: 1.2±0.5 vs. 0.7±0.2; SUVₘₑₐₙ: 0.6±0.3 vs. 0.4±0.1). Thus, one can suppose that a certain instability of SiFA-compounds is manageable in clinical translation.

### 3. CONCLUSION

Aim of this study was the development and investigation of a novel prosthetic group for ¹⁸F-labeling preserving the beneficial labeling properties and sufficient stability of the gold-standard (4-SiFA)Bz-moiety but contrarily offering an alternative by modifications leading to ligands with superior pharmacokinetic properties.

The requirement of highest priority for such an alternative is a substantial reduction of the excessive lipophilicity as a limiting factor induced by the so far used tBu₂FSi-Ar group. Furthermore, the SiFA-moiety displays a structural element also related to strong HSA binding while easy and effective modulation of binding strength plays a crucial role in optimizing a ligand's pharmacokinetic. Both could be successfully accomplished with the development of the (5-SiFA)Ip-moiety and readily modified derivatives thereof.

### 4. Material and Methods

### 4.1. General Information

The protected amino acid analogs were purchased from Bachem (Bubendorf, Switzerland), Carbolution Chemicals (St. Ingbert, Germany) or Iris Biotech (Marktredwitz, Germany). The 2-chlorotritylchloride (2-CTC) resin was obtained from Sigma-Aldrich (Steinheim, Germany). Chematech (Dijon, France) delivered the chelators DOTA, DOTA-GA and derivatives thereof. All necessary solvents and other organic reagents were purchased from either, Alfa Aesar (Karlsruhe, Germany), Sigma-Aldrich (Steinheim, Germany), Fluorochem (Hadfield, United Kingdom) or VWR (Darmstadt, Germany). Solid phase synthesis of the peptides was carried out by manual operation using an Intelli-Mixer syringe shaker (Neolab, Heidelberg, Germany). Analytical and preparative reversed-phase high-pressure chromatography (RP-HPLC) was performed using Shimadzu gradient systems (Shimadzu, Neufahrn, Germany), each equipped with an SPD-20A UV/Vis detector (220 nm). A MultoKrom 100 C18 (150×4.6 mm, 5 μm particle size) column (CS Chromatographie Service, Langerwehe, Germany) was used for analytical measurements at a flow rate of 1 mL/min. Both specific gradients and the corresponding retention times t_{R} are cited in the text. Preparative RP-HPLC purification was done with a MultoKrom 100 C18 (250×20 mm, 5 μm particle size) column (CS Chromatographie Service, Langerwehe, Germany) at a constant flow rate of 10 mL/min. Analytical and preparative radio-RP-HPLC was performed using a MultoKrom 100 C18 (150×4.6 mm, 5 μm particle size) column or Multokrom 100 C18 (125×4.6 mm, 5 μm particle size) column (CS Chromatographie Service, Langerwehe, Germany). Radioactivity was detected through connection of the outlet of the UV-photometer to a HERM LB 500 NaI detector (Berthold Technologies, Bad Wildbad, Germany) or Flowstar² LB 514 (Berthold Technologies, Bad Wildbad, Germany). Reversed-phase high performance flash chromatography (RP-HPFC) was performed on a Biotage® SP HPFC System (Biotage, Charlottesville, VA USA) using Biotage SNAP cartridges (KP-C18-HS, 12 g). Eluents for all HPLC operations were water (solvent A) and acetonitrile (solvent B), both containing 0.1% trifluoroacetic acid. Electrospray ionization-mass spectra for characterization of the substances were acquired on an expression^{L} CMS mass spectrometer (Advion, Harlow, United Kingdom). NMR spectra were recorded on a Bruker (Billerica, United States) AVHD-300, AVHD-400 or AVHD-500 spectrometer at 300 K. Chemical shifts are given in δ-values [ppm] and are referenced to the residual proton signal of the used deuterated solvent, or in cases of no solvent signal, e.g., for ¹⁹F or ²⁹Si NMR spectra, the shifts are calibrated to the internal standards CFCl₃ and tetramethylsilane, respectively. pH values were measured with a SevenEasy pH-meter (Mettler Toledo, Gießen, Germany). Activity quantification was performed using a 2480 WIZARD² automatic g-counter (PerkinElmer, Waltham, United States) or a CRCR-55tR dose calibrator (Mirion Technologies, Ramsey, NJ, USA). Radio-thin layer chromatography (TLC) was carried out with a Scan-RAM detector (LabLogic Systems, Sheffield, United Kingdom).

### 4.2. Solid Phase Peptide Synthesis

### 4.2.1. General Procedures (GPs)

### Standard 2-CTC Resin Loading (GP1a)

Loading of the 2-chlorotritylchloride (2-CTC) resin with a Fmoc-protected amino acid (AA) was carried out by stirring a solution of the 2-CTC-resin (1.60 mmol/g) and Fmoc-AA-OH (1.5 eq.) in DMF with DIPEA (3.0 eq.) at room temperature for 2 h. Remaining 2-chlorotritylchloride was capped by the addition of methanol (2 mL/g resin) for 15 min. Subsequently the resin was filtered and washed with DMF (8×5 mL/g resin) and DCM (3×5 mL/g resin) and dried in vacuo. Final loading l of Fmoc-AA-OH was determined by the following equation:

$$l [ \frac{m mol}{g} ] = \frac{( {m}_{2} - {m}_{1} ) \times 1000}{( {M}_{w} - {M}_{HCl} ) {m}_{2}}$$

$${m}_{2} = mass of loaded resin [ g ]$$

$${m}_{1} = mass of unloaded resin [ g ]$$

$${M}_{w} = molecular weight of AA [ g / mol ]$$

$${M}_{HCl} = molecular weight of HCl [ g / mol ]$$

### Standard H-Rink Amide ChemMatrix® Re,

Loading of the H-Rink amide ChemMatrix® resin was achieved using the procedure for amide bond formation (GP2). The used equivalents were referred to the initial resin loading given by the manufacturer (1.0 eq.=0.5 mmol/g resin). NOTE: Except for the Dde-deprotection step, in every GP, NMP was applied in the place of DMF when using the H-Rink amide ChemMatrix®-resin.

### Alternative 2-CTC Resin Loading (GP1c)

Loading of the 2-chlorotritylchloride (2-CTC) resin (GP1a) was carried out by stirring a solution of the 2-CTC-resin (1.60 mmol/g) and R—CO₂H (1.5 eq.) in DCM with DIPEA (3.0 eq.) at room temperature overnight (18h). Remaining 2-chlorotritylchloride was capped by the addition of methanol (2 mL/g resin) for 15 min. Subsequently the resin was filtered and washed with DCM (8×5 mL/g resin) and dried in vacuo. Final loading of the compound was determined in analogy to GP1a.

### Amide Bond Formation by Coupling to a Resin-Bound Amine (GP2a)

For conjugation of a building block to the resin-bound peptide, a mixture of TBTU (1.5 eq.) with HOAt (1.5 eq.) was used for pre-activation of the carboxylic acid (1.5 eq.) with DIPEA (4.5 eq.) or, in the case of pre-activating diamino propionic acid and derivatives thereof, 2,4,6-trimethylpyridine (6.0 eq.) as a base in DMF (10 mL/g resin). After 10 min at rt, the solution was added to the swollen resin. If not otherwise stated in the respective synthesis protocols, the conjugation step was carried out at rt for a period of 2 h. After the reaction, the resin was washed with DMF (6×5 mL/g resin).

### Amide Bond Formation by Coupling to a Resin-Bound Carboxylic Acid (GP2b)

For conjugation of a building block to the resin-bound peptide, a mixture of HATU (1.0 eq.) with HOAt (1.0 eq.) was used for pre-activation of the resin bound carboxylic (1.0 eq.) with DIPEA (3.0 eq.) for 30 min in DMF (10 mL/g resin). Next, the free amine (3.0 eq.) dissolved in DMF was added to the preactivated resin. If not otherwise stated in the respective synthesis protocols, the conjugation step was carried out for a period of 2 h at rt. After the reaction, the resin was washed with DMF (6×5 mL/g resin).

### Amide Bond Formation by Coupling Succinic Anhydride to a Resin-Bound Amine (GP2c)

For conjugation of succinic anhydride to a resin-bound peptide with an unprotected amine, a mixture of succinic anhydride (7.0 eq.) with DIPEA (7.0 eq.) in DMF (10 mL/g resin) was added to the resin. After 3 h at rt, the resin was washed with DMF (6×5 mL/g resin).

### Urea Bond Formation by Coupling to a Resin-Bound Amine (GP2d)

For binding motifs deviant from Glu-urea-Glu compounds, the synthesis of the Glu-urea-X compound, e.g., Glu-urea-Lys, was carried out in a similar way as an on-resin approach. A solution of (S)-di-tert-butyl-2-(1H-imidazole-1-carboxamido)pentanedioate in 1,2-dichloroethane (DCE, 5 mL/g resin) was added to the resin-bound peptide. The suspension was cooled on ice for 30 min and TEA (3.0 eq.) was added. The suspension was heated to 40° C. and stirred gently overnight. After reaction, the resin was washed with DMF (6×5 mL/g resin).

### On-Resin Fmoc-Deprotection (GP3)

The resin-bound Fmoc-peptide was treated with 20% piperidine in DMF (v/v, 8 mL/g resin) for 5 min and subsequently for 15 min. Afterwards, the resin was washed thoroughly with DMF (8×5 mL/g resin).

On-Resin Dde-Deprotection with Absent Fmoc-Groups (GP4a)

The Dde-protected peptide was dissolved in a solution of 2% hydrazine monohydrate in DMF (v/v, 5 mL/g resin) and shaken for 20 min. After deprotection the resin was washed with DMF (8×5 mL/g resin).

On-Resin Dde-Deprotection with Present Fmoc-Groups (GP4b)

In the case of present Fmoc-groups, Dde-deprotection of peptides bound to the 2-CTC resin was performed by adding a solution of imidazole (0.92 g/g resin), hydroxylamine hydrochloride (1.26 g/g resin) in NMP (5.0 mL/g resin) and DMF (1.0 mL/g resin) for 3 h at room temperature (GP4b). For the Dde-deprotection of peptides bound to the H-Rink amide ChemMatrix®-resin, the same amount of DCM was used instead of DMF. After deprotection the resin was washed with DMF (8×5 mL/g resin).

Peptide Cleavage Off the Resin with Concomitant Cleavage of Acid-Labile Protecting Groups (GP5a)

To cleave the peptide from the resin with concomitant cleavage of acid-labile protecting groups (GP5a), the fully protected resin-bound peptide was dissolved in a mixture of TFA/TIPS/water (v/v/v; 95/2.5/2.5) and shaken for 30 min. The solution is filtered off and the resin is treated in the same way for another 30 min. Both filtrates were combined and stirred for additional 1-24 h at rt. Product formation was monitored by HPLC. After removing TFA under a stream of nitrogen, the residue was dissolved in a mixture of tert-butanol and water and freeze-dried.

### Peptide Cleavage Off the Resin Under Preservation of Acid-Labile Protecting Groups (GP5b)

To cleave the peptide from the resin under preservation of acid-labile protecting groups (GP5b), the fully protected resin-bound peptide was dissolved in a mixture of 1,1,1,3,3,3-hexafluoro-2-propanol/DCM (v/v; 1/4) and shaken for 1 h. The solution was filtered off and the resin was treated in the same way for another 1 h. Both filtrates were combined and concentrated under a stream of nitrogen. The residue was dissolved in a mixture of tert-butanol and water and freeze-dried.

### On-Resin Allyl-/Alloc-Deprotection (GP6)

The allyl-/alloc-protected, resin-bound peptide was dissolved in a mixture of phenylsilane (24 eq) and Pd(PPh₃)₄ (0.1 eq.) in DCM (5 mL/g resin) and kept dark during the reaction for 20 min. The solution was filtered off and the resin was treated in the same way for another 20 min. To remove residual black palladium, the resin was washed alternately with sodium diethyldithiocarbamate in DMF (0.5 wt %, 5 mL/g resin) and 0.5% DIPEA in DMF (5 mL/g resin), each solution 3×5 min. Afterwards the resin was further washed with DMF (8×5 mL/g resin).

p-Nosyl-Protection (GP7)

The resin bound peptide was dissolved in a solution of p-Nosylchloride (5.0 eq.) and 2,4,6-Collidine (10.0 eq.) in NMP (10 mL/g resin) and shaken for 30 min at rt. After deprotection, the resin was washed with NMP (6×10 mL/g resin).

p-Nosyl-Deprotection (GP8)

A solution of DBU (5.0 eq.) in NMP (10 mL/g resin) was added to the p-Nosyl protected resin-bound peptide. After incubation over 5 min, p-Mercaptoethanol (10 eq.) was added and shaken for 15 min at rt. The procedure was repeated once. Afterwards, the resin was washed with NMP (6×10 mL/g resin).

### N-Methylation (GP9)

The resin was suspended in a solution of PPh₃ (0.3 M in anhydrous THF, 5.0 eq.) and MeOH (10 eq.) and the resin was shaken for 1 min at RT. Thereafter, DIAD (5.0 eq.) was added and the reaction mixture was shaken over 30 min at rt. After repetition of the previous procedure, the resin was washed with NMP (6×10 mL/g resin).

### Amide Bond Formation in Solution (GP10)

For conjugation of a building block to the unbound peptide, a mixture of HATU (1.1 eq.) with HOAt (1.1 eq.) was used for pre-activation of the carboxylic (1.1 eq.) with DIPEA (3.0 eq.) as a base in DMF (10 mL/g resin). After 5 min at rt, the mixture was added to free amine peptide dissolved in DMF. If not otherwise stated in the respective synthesis protocols, the conjugation step was carried out at rt for a period of 2 h. After the reaction, the solvent was removed under reduced pressure.

### Fmoc-Deprotection in Solution (GP11)

The unbound Fmoc-peptide was treated with 20% piperidine in DMF (v/v) for 20 min before removing the solvent under reduced pressure.

### Cyclization of Side Chain Protected Peptides in Solution (GP12)

To a solution of HATU (0.1 eq) and DIPEA (6.0 eq) in DMF (100 μL/μmol Pep), Pep. (1.0 eq) in DMF (100 μL/μmol) and HATU (3.0 eq) in DMF (100 μL/μmol Pep) were added dropwise each under vigorous stirring. Once the addition was completed, the reaction was stirred for additional 15 minutes before removing the solvent under reduced pressure.

### Removal of Acid-Labile Side Chain Protecting Groups in Solution (GP13)

The peptide was dissolved in a solution of TFA and water (95:5) (v:v), and stirred for 1-2 hours before removing the solvents under nitrogen flow. When bearing Trt-protecting groups a solution of TFA/H₂O/TIPS (v/v/v=95:2.5:2.5) was used.

### Allyl-Deprotection in Solution (GP14)

The allyl-protected peptide was dissolved in a mixture of phenylsilane (24 eq.) and Pd(PPh₃)₄ (0.1 eq.) in DCM (5 mL/g resin) and kept dark during the reaction for 1 hour at rt. The solvent was removed under reduced pressure. If not stated otherwise, residual Pd was quenched with the addition of TFA/H₂O (v/v=95/5) inducing additional sidechain deprotection.

Acm-Deprotection with Concomitant Disulfide-Bridge Formation (GP15)

The resin-bound, Cys(Acm)-containing peptide was incubated with Tl(TFA)₃ (4.0 eq.) in DMF (8 mL/g resin). After 45 min at rt, the solution was discarded, and the procedure was repeated once with fresh solution. The resin is then washed with DMF (6×5 mL/g resin).

### 4.2.2. Synthesis of SiFA-Based Building Blocks

4-(Di-tert-butylfluorosilyl)benzoic Acid ((4-SiFA)Bz-OH)
(prepared according to Wurzer et al. with minor modifications(5))

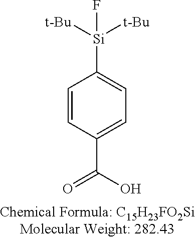 [SMILES: CC(C)(C)[Si](F)(c1ccc(C(=O)O)cc1)C(C)(C)C]

### ((4-Bromobenzyl)oxy)(tert-butyl)dimethylsilane (i)

To a stirred solution of 4-bromobenzylalcohol (4.68 g, 25.0 mmol, 1.0 eq.) in anhydrous DMF (70 mL) imidazole (2.04 g, 30.0 mmol, 1.2 eq.) and TBDMSCl (4.52 g, 30.0 mmol, 1.2 eq.) were added and the resulting mixture was stirred at room temperature (rt) for 16 h. The mixture was then poured into ice-cold H₂O (250 mL) and extracted with Et₂O (5×50 mL). The combined organic fractions were washed with sat. aq. NaHCO₃ (2×100 mL) and brine (100 mL), dried, filtered and concentrated in vacuo to give the crude product which was purified by flash column chromatography (silica, 5% EtOAc/petrol) to give i as a colorless oil (7.18 g, 95%). RP-HPLC (50 to 100% B in 15 min): t_{R}=15 min. K′=7.43.

### Di-tert-butyl[4-((tert-butyldimethylsilyloxy)methyl)phenyl]fluorosilane (ii)

At −78° C. under magnetic stirring, a solution of tBuLi in pentane (7.29 mL, 1.7 mol/L, 12.4 mmol 2.4 eq.) was added to a solution of ((4-bromobenzyl)oxy)(tert-butyl)dimethylsilane (i) (1.56 g, 5.18 mmol, 1.0 eq.) in dry THF (15 mL). After the reaction mixture had been stirred for 30 min at −78° C., the suspension obtained was added dropwise over a period of 30 min to a cooled (−78° C.) solution of di-tert-butyldifluorosilane (1.12 g, 6.23 mmol, 1.2 eq.) in dry THE (10 mL). The reaction mixture was allowed to warm to room temperature over a period of 12 h and then hydrolyzed with saturated aqueous NaCl solution (100 mL). The organic layer was separated and the aqueous layer was extracted with diethyl ether (3×50 mL). The combined organic layers were dried over magnesium sulfate and filtered. The filtrate was concentrated in vacuo to afford ii as a yellowish oil (1.88 g, 95%). It was used for subsequent reactions without further purification. RP-HPLC (50 to 100% B in 20 min): t_{R}=19 min. K′=9.67.

### 4-(Di-tert-butylfluorosilanyl)benzyl Alcohol (iii)

A catalytic amount of concentrated aqueous HCl (0.5 mL) was added to a suspension of ii (1.88 g, 4.92 mmol, 1.0 eq.) in methanol (50 mL). The reaction mixture was stirred for 18 h at room temperature and then the solvent and the volatiles were removed under reduced pressure. The residue was redissolved in diethyl ether (40 mL) and the solution was washed with saturated aqueous NaHCO₃ solution. The aqueous layer was extracted with diethyl ether (3×50 mL). The combined organic layers were dried over magnesium sulfate and filtered. The filtrate was concentrated in vacuo to afford iii as a yellowish oil (1.29 g, 98%) that solidified. The product was used without further purification. RP-HPLC (50 to 100% B in 15 min): t_{R}=8.2 min. K′=3.61.

### 4-(Di-tert-butylfluorosilyl)benzoic acid ((4-SiFA)Bz-OH, iv)

At rt, 5.8 g KMnO₄ (36.7 mmol, 1.5 eq.) were dissolved in H₂O and added to a solution of of iii (6.61 g, 24.6 mmol, 1.0 eq.), tert-butanol (65 mL), dichloromethane (9 mL), and 1.25 M NaH₂PO₄—H₂O buffer (36 mL) at pH 4.0-4.5. After the mixture had been stirred for 25 min, it was cooled on ice for 10 min, whereupon excess KMnO₄ (7.8 g, 49.2 mmol, 2.0 eq.) was added. The reaction mixture was stirred for 2 h on ice and subsequently allowed to warm to rt for 30 min. The reaction was then quenched by the addition of saturated aqueous Na₂SO₃ solution (50 mL). Upon addition of 2 M aqueous HCl, all of the MnO₂ dissolved. The resulting solution was extracted with diethyl ether (3×100 mL). The combined organic layers were washed with saturated aqueous NaHCO₃ solution, dried over MgSO₄, filtered, and concentrated under reduced pressure to provide a white solid, which was purified by recrystallization from Et₂O/n-hexane (1:3, for 12 h) to give iv (2.57 g, 37%). ¹H NMR (300 MHz, CDCl₃): δ [ppm]=8.10 (d, 2H³J(¹H,¹H)=8.1 Hz; Hₘ), 7.74 (d, 2H, ³J(¹H,¹H)=8.1 Hz; Ho), 1.07 (s, 18H; CCH₃); ¹³C{¹H}NMR (101 MHz, DMSO-D₆): δ [ppm]=167.2 (s; COOH), 138.3 (d, ²J(¹³C,¹⁹F)=14 Hz; Cp), 133.8 (d, ³J(¹³C,¹⁹F)=4 Hz; Cₘ), 132.1 (s; Cᵢ), 128.3 (s; Cₒ), 26.9 (s; CCH₃), 19.7 (d, ²J(¹³C,¹⁹F)=12 Hz; CCH₃); ¹⁹F{²⁹Si}NMR (376 MHz, DMSO-De): δ [ppm]=−187.2; ²⁹Si{¹H}INEPT NMR (79 MHz, DMSO-De): δ [ppm]=14.1 (d, ¹J(¹⁹F,²⁹Si)=299 Hz). RP-HPLC (50 to 100% B in 15 min): t_{R}=8.5 min. K′=3.78. ESI-MS (positive): calculated monoisotopic mass (C₁₅H₂₃FO₂Si): 282.15. found: m/z=283.2 [M+H]⁺, 265.2 [M−H₂O+H]⁺.

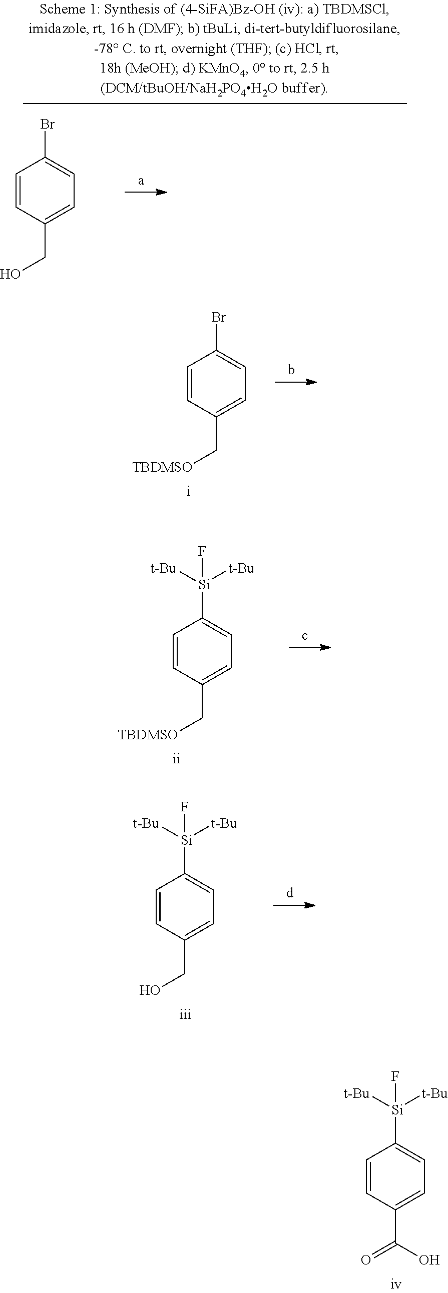 [SMILES: OCc1ccc(Br)cc1 ; CC(C)(C)[Si](C)(C)OCc1ccc(Br)cc1 ; CC(C)(C)[Si](C)(C)OCc1ccc([Si](F)(C(C)(C)C)C(C)(C)C)cc1 ; CC(C)(C)[Si](F)(c1ccc(CO)cc1)C(C)(C)C ; CC(C)(C)[Si](F)(c1ccc(C(=O)O)cc1)C(C)(C)C]

### 3-(Di-tert-butylfluorosilyl)benzoic acid ((3-SiFA)Bz-OH)

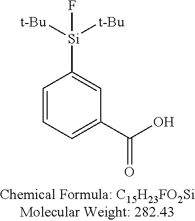 [SMILES: CC(C)(C)[Si](F)(c1cccc(C(=O)O)c1)C(C)(C)C]

### Di-tert-butylfluoro(3-tolyl)silane (i)

A solution of 342 mg 3-bromo-toluene (2.0 mmol, 1.0 eq.) in 6 mL dry THE was cooled to −78° C. and 2.59 mL of tBuLi (4.40 mmol, 1.6 M in pentane, 2.2 eq.) were added dropwise and stirred for 30 min at −78° C. The reaction mixture was then added to a solution of 397 mg of di-tert-butyldifluorosilane (2.20 mmol, 1.1 eq.) in 4.0 mL THF at −78° C. and stirred overnight allowing to warm to rt under pressure control. The reaction was stopped by addition of 40 mL brine. The aqueous layer was extracted with Et₂O (3×20 mL), combined organic phases were dried over MgSO₄ and the solvent was removed under reduced pressure to obtain the crude product as yellow oil. The mixture was used in the next step without further purification.

### 3-(Di-tert-butylfluorosilyl)benzoic acid ((3-SiFA)Bz-OH, ii)

To a solution of the complete crude product (i) (assumption: 2.0 mmol, 1.0 eq.) in 6.4 mL tBuOH/DCM (v/v=7/1) were added 8.1 mL of a NaH₂PO₄·H₂O solution (20.0 mmol, 1.25 M in H₂O, 10.0 eq.). To the solution 1.89 g KMnO₄ (12.0 mmol, 6.0 eq.) were added at rt, the reaction was carefully heated stepwise to 75° C. and stirred for 24 h. The reaction was quenched by the addition of a saturated aqueous NaSO₃ solution (15 mL). Concentrated aqueous HCl (5 mL) was added to completely dissolve MnO₂. The solution was extracted with Et₂O (3×40 mL), the combined organic phases were dried over MgSO₄ and the solvent removed under reduced pressure. The crude product was purified by HPFC and lyophilized to obtain 140 mg 3-(di-tert-butylfluorosilyl)benzoic acid (0.5 mmol, 25% over 2 steps) as a colorless solid. ¹H NMR (400 MHz, CDCl₃): δ [ppm]=8.36 (s, 1H; H_{Ar-2}), 8.16 (dt, 1H, ³J(¹H,¹H)=7.8 Hz, ⁴J(¹H,¹H)=1.6 Hz; H_{Ar-6}), 7.86 (dt, 1H, ³J(¹H,¹H)=7.4 Hz, ⁴J(¹H,¹H)=1.3 Hz; H_{Ar-4}), 7.51 (t, 1H, ³J(¹H,¹H)=7.6 Hz; H_{Ar-5}), 1.08 (s, 18H; CCH₃); ¹³C{¹H}NMR (101 MHz, DMSO-D₆): δ [ppm]=167.3 (s; COOH), 137.8 (d, ³J(¹³C,¹⁹F)=4 Hz; C_{Ar-4}), 134.1 (d, ³J(¹³C,¹⁹F)=4 Hz; C_{Ar-2}), 133.1 (d, ²J(¹³C,¹⁹F)=14 Hz; C_{Ar-3}), 130.7 (s; C_{Ar-6}), 130.1 (s; C_{Ar-5}), 128.2 (s; C_{Ar-1}), 26.9 (s; CCH₃), 19.7 (d, ²J(¹³C,¹⁹F)=12 Hz; CCH₃); ¹⁹F{²⁹Si}NMR (376 MHz, DMSO-D₆): δ [ppm]=−187.3; ²⁹Si{¹H}INEPT NMR (79 MHz, DMSO-De): δ [ppm]=13.8 (d, ¹J(¹⁹F,²¹Si)=298 Hz). RP-HPLC (50 to 100% B in 15 min): t_{R}=9.2 min.

K′=4.17. ESI-MS (positive): calculated monoisotopic mass (C₁₅H₂₃FO₂Si): 282.15. found: m/z=283.3 [M+H]⁺, 266.3 [M−H₂O+H]⁺.

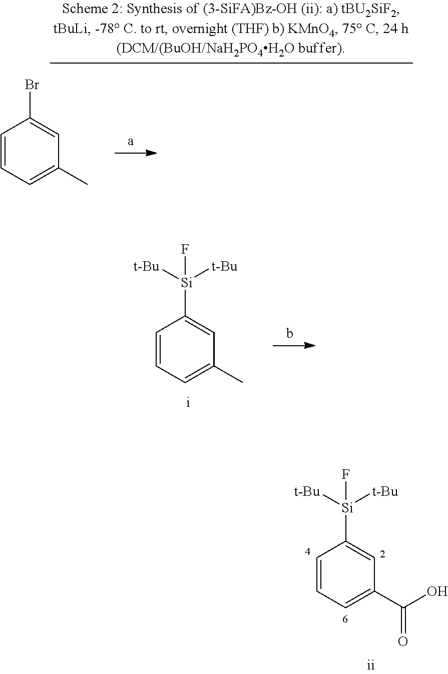

### 3-(Di-tert-butylfluorosilyl)-5-((allyloxy)carbonyl)benzoic acid (AIIO-(5-SiFA)Ip-OH)

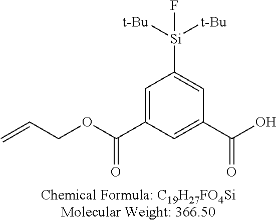 [SMILES: C=CCOC(=O)c1cc(C(=O)O)cc([Si](F)(C(C)(C)C)C(C)(C)C)c1]

### Di-tert-butyl(3,5-dimethylphenyl)fluorosilane (i)

A solution of 4.54 g 1-bromo-3,5-dimethylbenzene (25.1 mmol, 1.0 eq.) in 73.1 mL dry THF was cooled to −78° C. and 34.7 mL of tBuLi (55.5 mmol, 1.6 M in pentane, 2.2 eq.) were added dropwise and stirred for 30 min at −78° C. The reaction mixture was then added to a solution of 5.0 g of di-tert-butyldifluorosilane (27.7 mmol, 1.1 eq.) in 49.1 mL THF at −78° C. and stirred overnight while allowing to warm to rt under pressure control. The reaction was stopped by addition of 100 mL brine. The aqueous layer was extracted with Et₂O (3×100 mL), combined organic phases were dried over MgSO₄ and the solvent was removed under reduced pressure to obtain 6.6 g di-tert-butyl(3,5-dimethylphenyl)fluorosilane (24.8 mmol, 99%) as a colorless solid. ¹H NMR (500 MHz, CDCl₃): δ [ppm]=7.19 (s, 2H; Ho), 7.04 (s, 1H; Hₚ), 2.33 (s, 6H; CH₃), 1.06 (s, 18H; CCH₃). RP-HPLC (50 to 100% B in 15 min, 100% B for 10 min): t_{R}=16.4 min. K′=8.21.

### 5-(Di-tert-butylfluorosilyl)isophthalic acid ((5-SiFA)Ip-OH, ii)

To a solution of 1.1 g di-tert-butyl(3,5-dimethylphenyl)fluorosilane (i) (4.0 mmol, 1.0 eq.) in 16.8 mL tBuOH/DCM (v/v=3.5/1) were added 16.0 mL of a NaH₂PO₄·H₂O solution (40.0 mmol, 2.5 M in H₂O, 10.0 eq.). To the solution 7.6 g KMnO₄ (48.0 mmol, 12.0 eq.) were added at rt, the reaction was carefully heated stepwise to 75° C. and stirred for 24 h. The reaction was quenched by the addition of a saturated aqueous NaSO₃ solution (50 mL). Concentrated aqueous HCl (10 mL) was added to completely dissolve MnO₂. The solution was extracted with Et₂O (3×100 mL), the combined organic phases were dried over MgSO₄ and the solvent removed under reduced pressure to obtain ii (1.3 g, 4.0 mmol, 100%) as a colorless solid. ¹H NMR (400 MHz, DMSO-De): δ ppm=8.53 (t, 1H, ⁴J(¹H,¹H)=1.7 Hz; H_{Ar-2}), 8.32 (d, 2H, ⁴J(¹H,¹H)=1.6 Hz; H_{Ar-4,-6}), 1.03 (s, 18H; CH₃); ¹³C{¹H}NMR (101 MHz, DMSO-D₆): δ [ppm]=166.5 (s; COOH), 137.9 (d, ³J(¹³C,¹⁹F)=4 Hz; C_{Ar-4,-6}), 134.0 (d, ²J(¹³C,¹⁹F)=14 Hz; C_{Ar-5}), 131.4 (s; C_{Ar-2}), 130.8 (s; C_{Ar-1,-3}), 26.8 (s; CCH₃), 19.7 (d, ²J(¹³C,¹⁹F)=12 Hz; CCH₃); ¹⁹F{²⁹Si}NMR (376 MHz, DMSO-De): δ [ppm]=−187.1; ²⁹Si{¹H}INEPT NMR (79 MHz, DMSO-D₆): δ [ppm]=13.8 (d, ¹J(¹⁹F,²⁹Si)=299 Hz). RP-HPLC (50 to 100% B in 15 min): t_{R}=5.7 min. K′=2.20. ESI-MS (positive): calculated monoisotopic mass (C₁₆H₂₃FO₄Si): 326.13. found: m/z=327.2 [M+H]⁺, 309.2 [M−H₂O+H]⁺.

### 3-(Di-tert-butylfluorosilyl)-5-((allyloxy)carbonyl)benzoic acid (AIIO-(5-SiFA)Ip-OH, iii)

A suspension of 1.0 g 5-(di-tert-butylfluorosilyl)isophthalic acid (ii) (3.1 mmol, 1.0 eq.) and 1.3 g K₂CO₃ (9.2 mmol, 3.0 eq.) in 300 mL DMF was cooled to 0° C. and a solution of 185.0 mg Allylbromide (132.0 μL, 1.5 mmol, 0.5 eq.) in 10.0 mL DMF was slowly added over 20 min. The mixture was stirred overnight at rt. Subsequently, the mixture was filtered and all volatiles of the filtrate were removed under reduced pressure. The residue was dissolved in 100 mL HCl (1 M) and the aqueous layer was extracted with Et₂O (3×200 mL). The combined organic phases were dried over MgSO₄ and the solvent was removed under reduced pressure. The crude product was purified by HPFC and lyophilized to yield iii as colorless solid (250.9 mg, 684.6 mmol, 19%). ¹H NMR (400 MHz, DMSO-De): δ [ppm]=8.57 (t, 1H, ⁴J(¹H,¹H)=1.5 Hz; H_{Ar-6}), 8.36 (t, 1H, ⁴J(¹H,¹H)=1.5 Hz; H_{Ar-2}), 8.34 (t, 1H, ⁴J(¹H,¹H)=1.5 Hz; H_{Ar-4}), 6.07 (ddt, 1H, ³J(¹H,¹H)=17.3, 10.7, 5.5 Hz; CH₂—CH═CH₂), 5.41 (dd, 1H, ³J(¹H,¹H)=17.2 Hz, ²J(¹H,¹H)=1.7 Hz; CH₂—CH═CH₂ (E)), 5.30 (dd, 1H, ³J(¹H,¹H)=10.3 Hz, ²J(¹H,¹H)=1.6 Hz; CH₂—CH═CH₂ (Z)), 4.85 (d, 2H, ³J(¹H,¹H)=5.6 Hz; CH₂CH═CH₂), 1.03 (s, 18H; CCH₃); ¹³C{¹H}NMR (101 MHz, DMSO-D₆): δ [ppm]=166.3 (COOH), 164.6 (CO-OAII), 138.3 (d, ³J(¹³C,¹⁹F)=4 Hz; C_{Ar-4}), 137.6 (d, ³J(¹³C,¹⁹F)=4 Hz; C_{Ar-6}), 134.3 (d, ²J(¹³C,¹⁹F)=14 Hz; C_{Ar-5}), 132.4 (CH₂CH═CH₂), 131.2 (C_{Ar-2}), 131.0 (C_{Ar-1}), 129.7 (C_{Ar-3}), 118.2 (CH₂—CH═CH₂), 65.6 (CH₂—CH═CH₂), 26.8 (s; CCH₃), 19.7 (d, ²J(¹³C,¹⁹F)=12 Hz; CCH₃); ¹⁹F{²⁹Si}NMR (376 MHz, DMSO-D₆): δ [ppm]=−187.0; ²⁹Si{¹H}INEPT NMR (79 MHz, DMSO-D₆): δ [ppm]=13.8 (d, ¹J(¹⁹F,²⁹Si)=299 Hz). RP-HPLC (50 to 100% B in 15 min): t_{R}=10.4 min. K′=4.84.

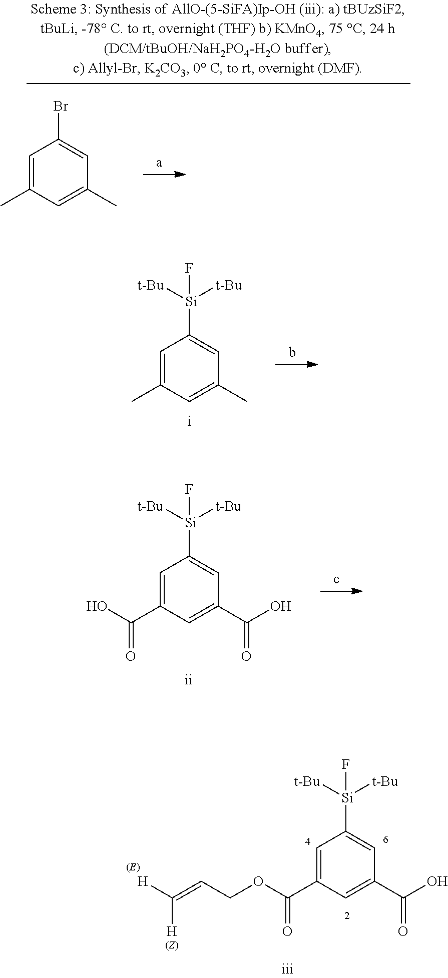

### 4.2.3. Synthesis of Model Peptides (MPs)

### (4-SiFA)Bz-Gly-L-Lys-OH (1a)

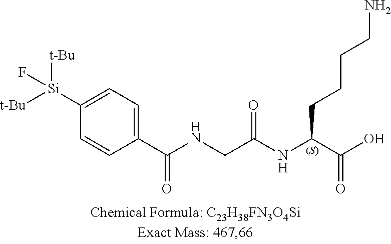

2-CTC-resin was loaded with Fmoc-L-Lys(Boc)-OH (GP1a). After Fmoc-deprotection (GP3), Fmoc-Gly-OH was coupled to the resin-bound amino acid (GP2a). After another Fmoc-deprotection (GP3), (4-SiFA)Bz-OH was conjugated to the free amine (GP2a). The peptide was cleaved from the resin under concomitant deprotection (GP5a) and isolated after lyophilization as colorless solid (77%). ¹⁹F{²⁹Si}NMR (376 MHz, DMSO-D₆): δ [ppm]=−187.17; ²⁹Si{¹H}INEPT NMR (79 MHz, DMSO-D₆): δ [ppm]=14.08 (d, ¹J(¹⁹F,²⁹Si)=299 Hz). RP-HPLC (30 to 90% B in 15 min): t_{R}=7.4 min. K′=3.16. ESI-MS (positive): calculated monoisotopic mass (C₂₃H₃₈FN₃O₄Si): 467.26. found: m/z=468.4 [M+H]⁺.

### (4-SiFA)Bz-Gly-L-Asp-OH (1b)

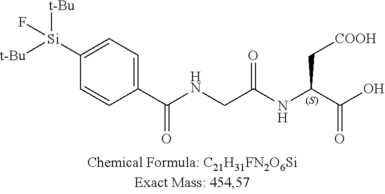

2-CTC-resin was loaded with Fmoc-L-Asp(tBu)-OH (GP1a). After Fmoc-deprotection (GP3), Fmoc-Gly-OH was coupled to the resin-bound amino acid (GP2a). After another Fmoc-deprotection (GP3), (4-SiFA)Bz-OH was conjugated to the free amine (GP2a). The peptide was cleaved from the resin under concomitant deprotection (GP5a) and isolated after lyophilization as colorless solid (86%). ¹⁹F{²⁹Si}NMR (376 MHz, DMSO-D₆): δ [ppm]=−187.17; ²⁹Si{¹H}INEPT NMR (79 MHz, DMSO-D₆): δ [ppm]=14.08 (d, ¹J(¹⁹F,²⁹Si)=298 Hz). RP-HPLC (30 to 90% B in 15 min): t_{R}=9.1 min. K′=4.11. ESI-MS (positive): calculated monoisotopic mass (C₂₁H₃₁FN₂O₆Si): 454.19. found: m/z=455.3 [M+H]⁺.

### (4-SiFA)Bz-Gly-L-Tyr-OH (1c)

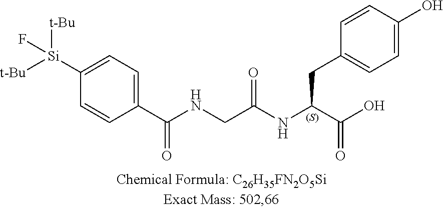

2-CTC-resin was loaded with Fmoc-L-Tyr(OtBu)-OH (GP1a). After Fmoc-deprotection (GP3), Fmoc-Gly-OH was coupled to the resin-bound amino acid (GP2a). After another Fmoc-deprotection (GP3), (4-SiFA)Bz-OH was conjugated to the free amine (GP2a). The peptide was cleaved from the resin under concomitant deprotection (GP5a) and isolated after lyophilization as colorless solid (90%). ¹⁹F{²⁹Si}NMR (376 MHz, DMSO-De): δ [ppm]=−187.12; ²⁹Si{¹H}INEPT NMR (79 MHz, DMSO-D₆): δ [ppm]=14.09 (d, ¹J(¹⁹F,²⁹Si)=298 Hz). RP-HPLC (30 to 90% B in 15 min): t_{R}=10.2 min. K′=4.73. ESI-MS (positive): calculated monoisotopic mass (C₂₆H₃₅FN₂O₅Si): 502.23. found: m/z=503.3 [M+H]⁺.

### (3-SiFA)Bz-Gly-L-Lys-OH (2a)

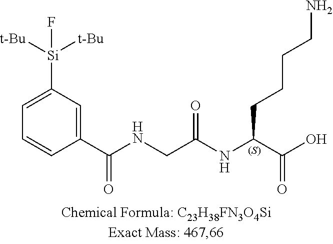

2-CTC-resin was loaded with Fmoc-L-Lys(Boc)-OH (GP1a). After Fmoc-deprotection (GP3), Fmoc-Gly-OH was coupled to the resin-bound amino acid (GP2a). After another Fmoc-deprotection (GP3), (3-SiFA)Bz-OH was conjugated to the free amine (GP2a). The peptide was cleaved from the resin under concomitant deprotection (GP5a) and isolated after lyophilization as colorless solid (95%). ¹⁹F{²⁹Si}NMR (376 MHz, DMSO-D₆): δ [ppm]=−186.90; ²⁹Si{¹H}INEPT NMR (79 MHz, DMSO-D₆): δ [ppm]=13.98 (d, ¹J(¹⁹F,²⁹Si)=298 Hz). RP-HPLC (20 to 90% B in 15 min): t_{R}=11.3 min. K′=5.35. ESI-MS (positive): calculated monoisotopic mass (C₂₃H₃₈FN₃O₄Si): 467.26. found: m/z=468.5 [M+H]⁺.

### (3-SiFA)Bz-Gly-L-Asp-OH (2b)

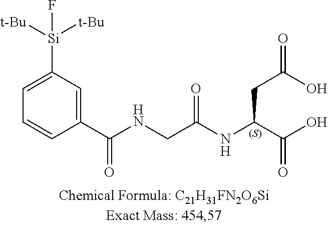

2-CTC-resin was loaded with Fmoc-L-Asp(OtBu)-OH (GP1a). After Fmoc-deprotection (GP3), Fmoc-Gly-OH was coupled to the resin-bound amino acid (GP2a). After another Fmoc-deprotection (GP3), (3-SiFA)Bz-OH was conjugated to the free amine (GP2a). The peptide was cleaved from the resin under concomitant deprotection (GP5a) and isolated after lyophilization as colorless solid (79%). ¹⁹F{²⁹Si}NMR (376 MHz, DMSO-De): δ [ppm]=−186.90; ²⁹Si{¹H}INEPT NMR (79 MHz, DMSO-De): δ [ppm]=13.98 (d, ¹J(¹⁹F,²⁹Si)=298 Hz). RP-HPLC (20 to 90% B in 15 min): t_{R}=10.3 min. K′=4.79. ESI-MS (positive): calculated monoisotopic mass (C₂₁H₃₁FN₂O₆Si): 454.19. found: m/z=455.4 [M+H]⁺.

### (3-SiFA)Bz-Gly-L-Tyr-OH (2c)

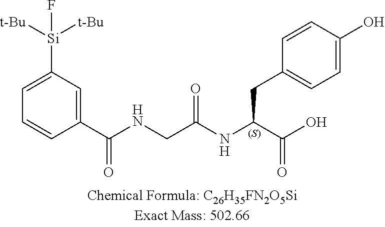

2-CTC-resin was loaded with Fmoc-L-Tyr(OtBu)-OH (GP1a). After Fmoc-deprotection (GP3), Fmoc-Gly-OH was coupled to the resin-bound amino acid (GP2a). After another Fmoc-deprotection (GP3), (3-SiFA)Bz-OH was conjugated to the free amine (GP2a). The peptide was cleaved from the resin under concomitant deprotection (GP5a) and isolated after lyophilization as colorless solid (77%). ¹⁹F{²⁹Si}NMR (376 MHz, DMSO-D₆): δ [ppm]=−186.86; ²⁹Si{¹H}INEPT NMR (79 MHz, DMSO-D₆): δ [ppm]=13.99 (d, ¹J(¹⁹F,²⁹Si)=298 Hz). RP-HPLC (20 to 90% B in 15 min): t_{R}=8.8 min. K′=3.94. ESI-MS (positive): calculated monoisotopic mass (C₂₆H₃₅FN₂O₅Si): 502.23. found: m/z=503.8 [M+H]⁺.

### (5-SiFA)Ip-Gly-L-Lys-OH (3a)

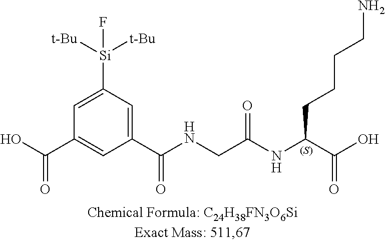

2-CTC-resin was loaded with Fmoc-L-Lys(Boc)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6), the peptide was cleaved from the resin under concomitant deprotection (GP5a) and isolated after HPFC-based purification and lyophilization as colorless solid (25%). ¹⁹F{²⁹Si}NMR (376 MHz, DMSO-D₆): δ [ppm]=−186.66; ²⁹Si{¹H}INEPT NMR (79 MHz, DMSO-D₆): δ [ppm]=13.96 (d, ¹J(¹⁹F,²⁹Si)=299 Hz). RP-HPLC (20 to 90% B in 15 min): t_{R}=9.6 min. K′=4.39. ESI-MS (positive): calculated monoisotopic mass (C₂₄H₃₈FN₃O₆Si): 511.25. found: m/z=512.1 [M+H]⁺.

### (5-SiFA)Ip-Gly-L-Asp-OH (3b)

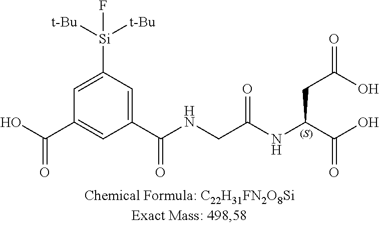

2-CTC-resin was loaded with Fmoc-L-Asp(OtBu)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6), the peptide was cleaved from the resin under concomitant deprotection (GP5a) and isolated after HPFC-based purification and lyophilization as colorless solid (38%). ¹⁹F{²⁹Si}NMR (376 MHz, DMSO-D₆): δ [ppm]=−186.65; ²⁹Si{¹H}INEPT NMR (79 MHz, DMSO-D₆): δ [ppm]=13.94 (d, ¹J(¹⁹F,²⁹Si)=299 Hz). RP-HPLC (20 to 90% B in 15 min): t_{R}=8.6 min. K′=3.83. ESI-MS (positive): calculated monoisotopic mass (C₂₂H₃₁FN₂O₈Si): 498.18. found: m/z=499.1 [M+H]⁺.

### (5-SiFA)Ip-Gly-L-Tyr-OH (3c)

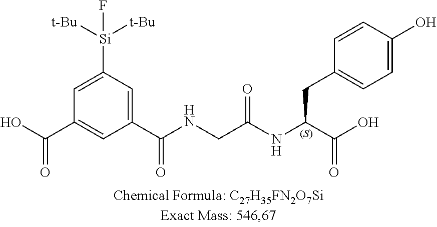

2-CTC-resin was loaded with Fmoc-L-Tyr(OtBu)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6), the peptide was cleaved from the resin under concomitant deprotection (GP5a) and isolated after HPFC-based purification and lyophilization as colorless solid (18%). ¹⁹F{²⁹Si}NMR (376 MHz, DMSO-De): δ [ppm]=−186.62; ²⁹Si{¹H}INEPT NMR (79 MHz, DMSO-D₆): δ [ppm]=13.96 (d, ¹J(¹⁹F,²⁹Si)=299 Hz). RP-HPLC (20 to 90% B in 15 min): t_{R}=9.6 min. K′=4.39. ESI-MS (positive): calculated monoisotopic mass (C₂₇H₃₅FN₂O₇Si): 546.22. found: m/z=547.0 [M+H]⁺.

### HO-L-Lys-(5-SiFA)Ip-Gly-L-Lys-OH (4a)

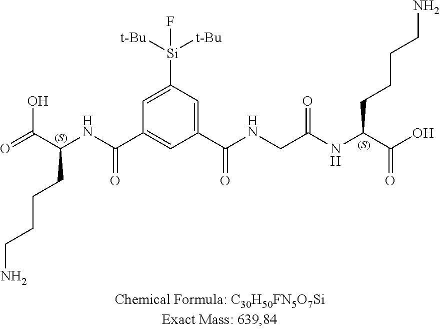

2-CTC-resin was loaded with Fmoc-L-Lys(Boc)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6) and H-L-Lys(Boc)-OtBu-HCl was coupled to the deprotected carboxylic acid with a modified GP2, pre-activating the resin-coupled peptide for 20 min under incubation with HOAt (1.5 eq.), TBTU (1.5 eq.) and DIPEA (6.0 eq.) in DMF followed by the addition of the amino acid (1.5 eq.) dissolved in DMF at rt. The peptide was cleaved from the resin under concomitant deprotection (GP5a) and isolated after HPFC-based purification and lyophilization as colorless solid (16%). ¹⁹F{²⁹Si}NMR (376 MHz, DMSO-De): δ [ppm]=−186.19; ²⁹Si{¹H}INEPT NMR (79 MHz, DMSO-D₆): δ [ppm]=14.09 (d, ¹J(¹⁹F,²⁹Si)=299 Hz). RP-HPLC (20 to 90% B in 15 min): t_{R}=5.7 min. K′=2.20. ESI-MS (positive): calculated monoisotopic mass (C₃₀H₅₀FN₅O₇Si): 639.35. found: m/z=640.6 [M+H]⁺.

### HO-L-Lys-(5-SiFA)Ip-Gly-L-Asp-OH (4b)

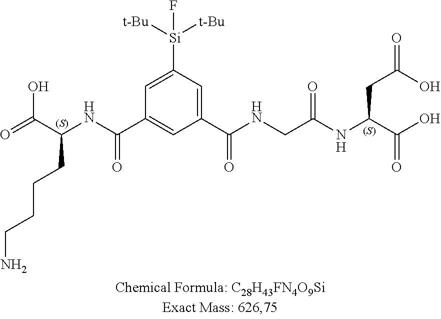

2-CTC-resin was loaded with Fmoc-L-Asp(OtBu)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6) and H-L-Lys(Boc)-OtBu-HCl was coupled to the deprotected carboxylic acid with a modified GP2, pre-activating the resin-coupled peptide for 20 min under incubation with HOAt (1.5 eq.), TBTU (1.5 eq.) and DIPEA (6.0 eq.) in DMF followed by the addition of the amino acid (1.5 eq.) dissolved in DMF at rt. The peptide was cleaved from the resin under concomitant deprotection (GP5a) and isolated after HPFC-based purification and lyophilization as colorless solid (21%). ¹⁹F{²⁹Si}NMR (376 MHz, DMSO-D₆): δ [ppm]=−186.22; ²⁹Si{¹H}INEPT NMR (79 MHz, DMSO-D₆): δ [ppm]=14.09 (d, ¹J(¹⁹F,²⁹Si)=299 Hz). RP-HPLC (20 to 90% B in 15 min): t_{R}=6.6 min. K′=2.71. ESI-MS (positive): calculated monoisotopic mass (C₂₈H₄₃FN₄O₉Si): 626.28. found: m/z=627.5 [M+H]⁺.

### HO-L-Lys-(5-SiFA)Ip-Gly-L-Tyr-OH (4c)

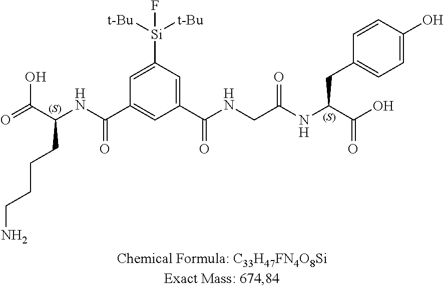

2-CTC-resin was loaded with Fmoc-L-Tyr(OtBu)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6) and H-L-Lys(Boc)-OtBu-HCl was coupled to the deprotected carboxylic acid with a modified GP2, pre-activating the resin-coupled peptide for 20 min under incubation with HOAt (1.5 eq.), TBTU (1.5 eq.) and DIPEA (6.0 eq.) in DMF followed by the addition of the amino acid (1.5 eq.) dissolved in DMF at rt. The peptide was cleaved from the resin under concomitant deprotection (GP5a) and isolated after HPFC-based purification and lyophilization as colorless solid (9%). ¹⁹F{²⁹Si}NMR (376 MHz, DMSO-D₆): δ [ppm]=−186.28; ²⁹Si{¹H}INEPT NMR (79 MHz, DMSO-De): δ [ppm]=14.06 (d, ¹J(¹⁹F,²⁹Si)=299 Hz). RP-HPLC (20 to 90% B in 15 min): t_{R}=7.3 min. K′=3.10. ESI-MS (positive): calculated monoisotopic mass (C₃₃H₄₇FN₄O₈Si): 674.31. found: m/z=675.5 [M+H]⁺.

### HO-L-Glu-(5-SiFA)Ip-Gly-L-Lys-OH (5a)

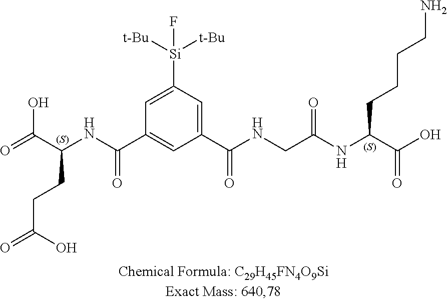

2-CTC-resin was loaded with Fmoc-L-Lys(Boc)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6) and H-L-Glu(OtBu)-OtBu-HCl was coupled to the deprotected carboxylic acid with a modified GP2, pre-activating the resin-coupled peptide for 20 min under incubation with HOAt (1.5 eq.), TBTU (1.5 eq.) and DIPEA (6.0 eq.) in DMF followed by the addition of the amino acid (1.5 eq.) dissolved in DMF at rt. The peptide was cleaved from the resin under concomitant deprotection (GP5a) and isolated after HPFC-based purification and lyophilization as colorless solid (12%). RP-HPLC (20 to 90% B in 15 min): t_{R}=6.7 min. K′=2.76. ESI-MS (positive): calculated monoisotopic mass (C₂₉H₄₅FN₄O₉Si): 640.29. found: m/z=640.7 [M+H]⁺.

### HO-L-Glu-(5-SiFA)Ip-Gly-L-Asp-OH (5b)

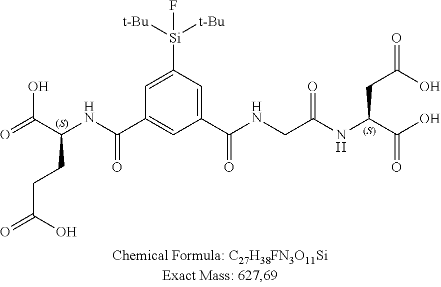

2-CTC-resin was loaded with Fmoc-L-Asp(OtBu)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6) and H-L-Glu(OtBu)-OtBu-HCl was coupled to the deprotected carboxylic acid with a modified GP2, pre-activating the resin-coupled peptide for 20 min under incubation with HOAt (1.5 eq.), TBTU (1.5 eq.) and DIPEA (6.0 eq.) in DMF followed by the addition of the amino acid (1.5 eq.) dissolved in DMF at rt. The peptide was cleaved from the resin under concomitant deprotection (GP5a) and isolated after HPFC-based purification and lyophilization as colorless solid (23%). RP-HPLC (20 to 90% B in 15 min): t_{R}=7.5 min. K′=3.21. ESI-MS (positive): calculated monoisotopic mass (C₂₇H₃₈FN₃O₁₁Si): 627.23. found: m/z=627.7 [M+H]⁺.

### HO-L-Glu-(5-SiFA)Ip-Gly-L-Tyr-OH (5c)

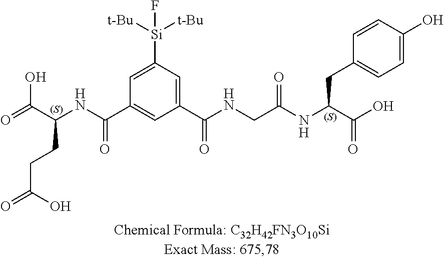

2-CTC-resin was loaded with Fmoc-L-Tyr(OtBu)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6) and H-L-Glu(OtBu)-OtBu-HCl was coupled to the deprotected carboxylic acid with a modified GP2, pre-activating the resin-coupled peptide for 20 min under incubation with HOAt (1.5 eq.), TBTU (1.5 eq.) and DIPEA (6.0 eq.) in DMF followed by the addition of the amino acid (1.5 eq.) dissolved in DMF at rt. The peptide was cleaved from the resin under concomitant deprotection (GP5a) and isolated after HPFC-based purification and lyophilization as colorless solid (15%). RP-HPLC (20 to 90% B in 15 min): t_{R}=8.3 min. K′=3.66. ESI-MS (positive): calculated monoisotopic mass (C₃₂H₄₂FN₃O₁₀Si): 675.26. found: m/z=675.7 [M+H]⁺.

### (5-SiFA)Ip-L-Lys-Gly-OH (3a*)

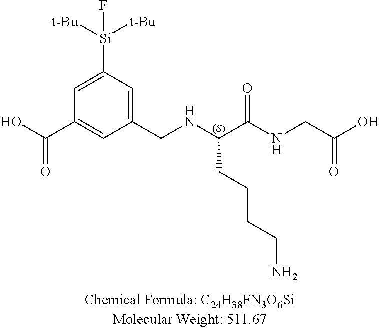

2-CTC-resin was loaded with Fmoc-Gly-OH (GP1a). After Fmoc-deprotection (GP3), Fmoc-L-Lys(Boc)-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6), the peptide was cleaved from the resin under concomitant deprotection (GP5a) and isolated after RP-HPLC-based purification and lyophilization as colorless solid (26%). RP-HPLC (20 to 90% B in 15 min): t_{R}=7.6 min. K′=3.27. ESI-MS (positive): calculated monoisotopic mass (C₂₄H₃₈FN₃O₆Si): 511.25. found: m/z=511.6 [M+H]⁺.

### HOOC-PEG₈-(5-SiFA)Ip-Gly-L-Lys-OH (6a)

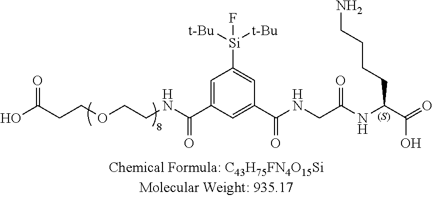

For the preparation of the model peptide 6a, a combined solution phase and solid phase peptide synthesis strategy was used. At first, the dipeptide H-Gly-L-Lys(Boc)-OtBu-TFA was synthesized by pre-activation of 178 mg Fmoc-Gly-OH (0.6 mmol, 1.2 eq.) with 82 mg HOAt (0.6 mmol, 1.2 eq.), 228 mg HATU (0.6 mmol, 1.2 eq.) in 5 mL DMF and addition of 510 μL DIPEA (3.0 mmol, 5.0 eq.). After 15 min at rt, 1 mL of a solution of 169 mg H-L-Lys(Boc)-OtBu (0.5 mmol, 1.0 eq.) in DMF was added and stirred for 4 h at rt. For subsequent Fmoc-deprotection with 30 vol.-% piperidine in DMF, 2.5 mL piperidine were added to the solution. After 30 min, the solvents were removed under reduced pressure at 60° C. HPFC-purification and subsequent lyophilization of the dipeptide yielded 184 mg of a colorless solid (78%). RP-HPLC (10 to 90% B in 15 min): t_{R}=8.9 min. K′=4.00. ESI-MS (positive): calculated monoisotopic mass (C₁₇H₃₃N₃O₅): 359.24. found: m/z=359.8 [M+H]⁺.

2-CTC-resin was loaded with Fmoc-NH-PEG₈-CH₂—CH₂—COOH (GP1a). After Fmoc-deprotection (GP3), AIIO-(5-SiFA)Ip-OH was conjugated to the free amine (GP2a). Allyl-deprotection was conducted (GP6) and H-Gly-L-Lys(Boc)-OtBu-TFA was coupled to the deprotected carboxylic acid with a modified GP2, pre-activating the resin-coupled peptide for 20 min under incubation with HOAt (1.5 eq.), TBTU (1.5 eq.) and DIPEA (6.0 eq.) in DMF followed by the addition of the amino acid (1.5 eq.) dissolved in DMF at rt. The peptide was cleaved from the resin under concomitant deprotection (GP5a) and isolated after RP-HPLC-based purification and lyophilization as colorless solid (25%). RP-HPLC (20 to 90% B in 15 min): t_{R}=7.5 min. K′=3.21. ESI-MS (positive): calculated monoisotopic mass (C₄₃H₇₅FN₄O₁₅Si): 934.50. found: m/z=934.4 [M+H]⁺.

### Gal6N-(5-SiFA)Ip-Gly-L-Lys-OH (7a)

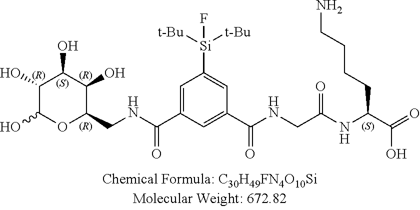

2-CTC-resin was loaded with Fmoc-L-Lys(Boc)-OH (GP1a). After Fmoc-deprotection (GP3), Fmoc-Gly-OH was coupled to the resin-bound amino acid (GP2a). After another Fmoc-deprotection (GP3), AIIO-(5-SiFA)Ip-OH was conjugated to the free amine (GP2a). Allyl-deprotection was conducted (GP6) and for the next coupling step a modified version of GP2 was used, pre-activating the resin-coupled peptide for 20 min under incubation with HOAt (1.5 eq.), TBTU (1.5 eq.) and DIPEA (6.0 eq.) in DMF followed by the addition of 6-amino-6-deoxy-1,2;3,4-di-O-isopropylidene-D-galactopyranoside (1.5 eq.) dissolved in DMF at rt. The peptide was cleaved from the resin under concomitant deprotection (GP5a) and isolated after RP-HPLC-based purification and lyophilization as colorless solid (17%). RP-HPLC (20 to 90% B in 15 min): t_{R}=5.8 min. K′=2.26. ESI-MS (positive): calculated monoisotopic mass (C₃₀H₄₉FN₄O₁₀Si): 672.32. found: m/z=672.8 [M+H]⁺.

### 4.2.4. Synthesis of Vector-Based Ligands

### Synthesis of GRPR Ligands

Fmoc-D-Dap(NH₂)-EDA-DOTA(tBu)₂-[α-Me-Trp⁸]MJ9(PG) (1)

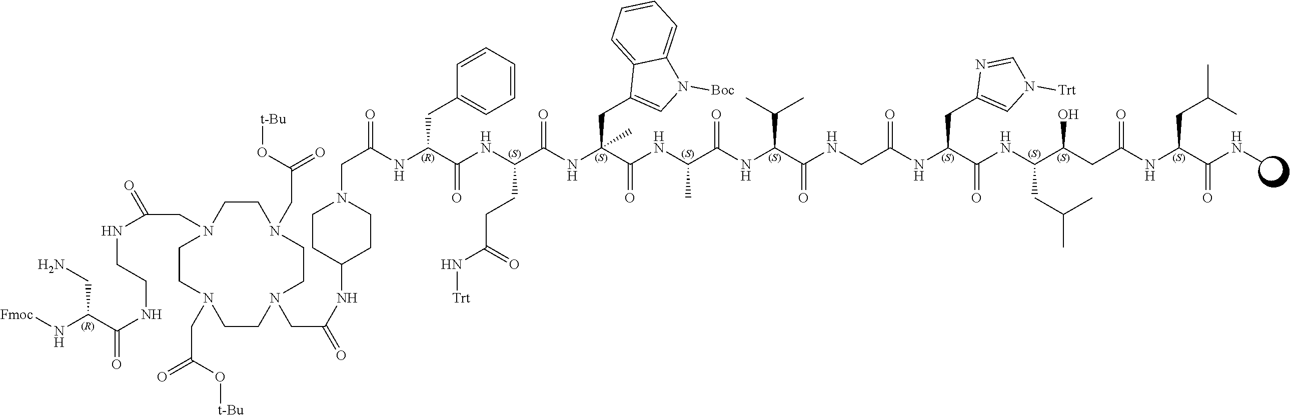

All GRPR-targeted compounds were synthesized by standard Fmoc-based SPPS (GP1 b, GP2a, GP3) using a H-Rink amide ChemMatrix® resin (35-100 mesh particle size, 0.4-0.6 mmol/g loading). The resin was loaded with Fmoc-L-Leu-OH according to GP1b. Subsequently, the amino acids Fmoc-Sta-OH, Fmoc-L-His(Trt)-OH, Fmoc-Gly-OH, Fmoc-L-Val-OH, Fmoc-L-Ala-OH, Fmoc-a-Me-L-Trp(Boc)-OH, Fmoc-L-Gln(Trt)-OH, Fmoc-D-Phe-OH and Fmoc-4-APipAc-OH were alternately coupled (GP2a) and Fmoc-deprotected (GP3). DOTA-di(tBu)-OH was coupled to the resin following GP2a. Fmoc-EDA-NH₂ was coupled in analogy to GP2b and Fmoc-deprotection was done following GP3. On-resin coupling of Fmoc-D-Dap(Dde)-OH was performed using 2,4,6-trimethylpyridine (6.0 eq.) as a base for its pre-activation (GP2a). The sidechain was Dde-deprotected under preservation of Fmoc (GP4b) to complete the resin-bound precursor used in the synthesis of the following GRPR ligands.

### DOTAGA-D-Dap[(4-SiFA)Bz]-EDA-DOTA-[α-Me-Trp⁸]MJ9 (2)

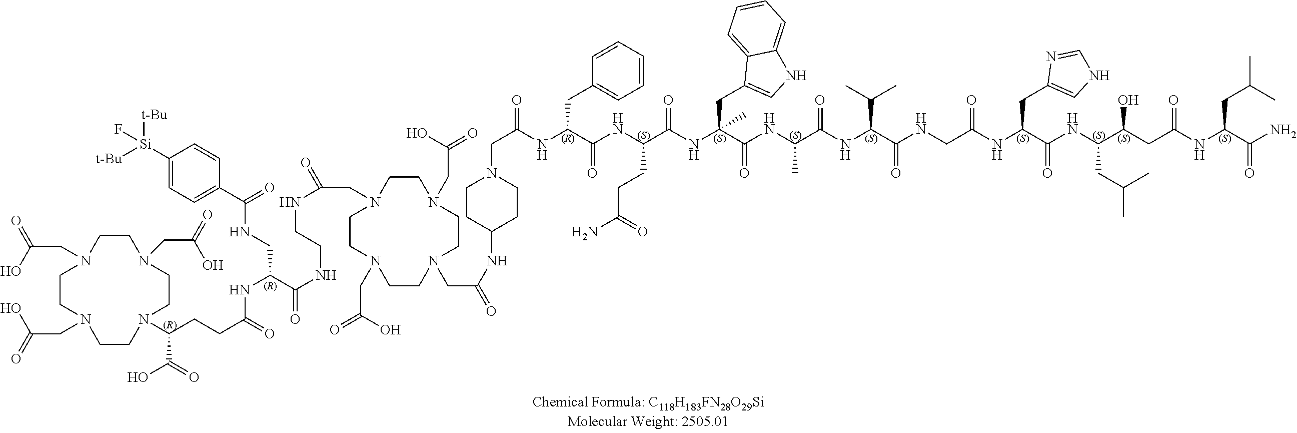

(4-SiFA)Bz-OH was coupled to the unprotected Dap-sidechain. Subsequently, the Fmoc protecting group was removed (GP3) and DOTAGA(tBu)₄ was coupled for a prolonged time of 6 h at rt (modified GP2a). The peptide was cleaved off the resin under concomitant deprotection (GP5a) and isolated after HPLC-based purification and lyophilization as colorless solid (8%). RP-HPLC (10 to 90% B in 15 min): t_{R}=11.6 min. K′=6.25. ESI-MS (positive): calculated monoisotopic mass (C₁₁₈H₁₈₃FN₂₈O₂₉Si): 2505.0. found: m/z=1253.7 [M+2H]²⁺, 836.3 [M+3H]³⁺, 627.5 [M+4H]⁴⁺.

### DOTAGA-D-Dap[(3-SiFA)Bz]-EDA-DOTA-[α-Me-Trp⁸]MJ9 (3)

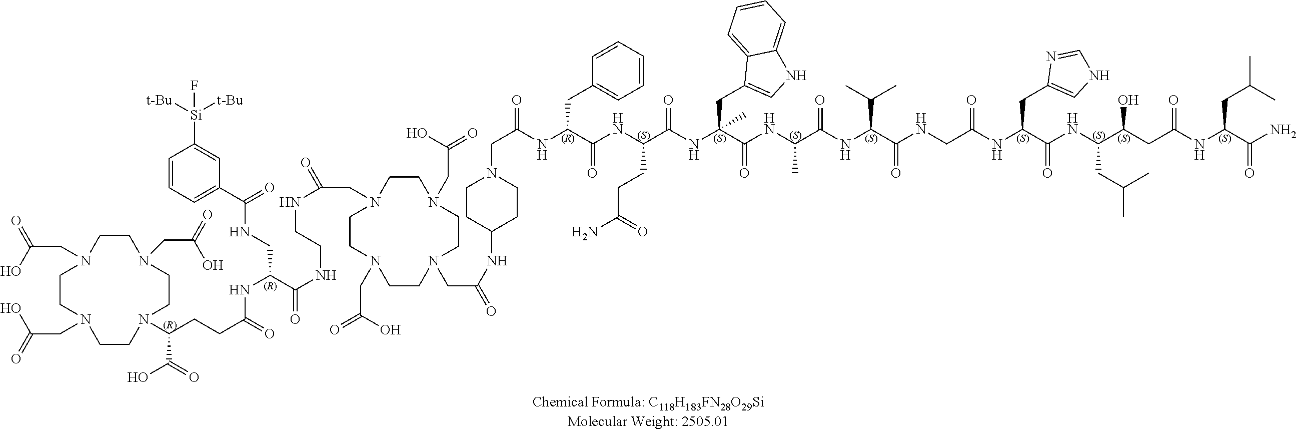

(3-SiFA)Bz-OH was coupled to the unprotected Dap-sidechain. Subsequently, the Fmoc protecting group was removed (GP3) and DOTAGA(tBu)₄ was coupled for a prolonged time of 6 h at rt (modified GP2a). The peptide was cleaved off the resin under concomitant deprotection (GP5a) and isolated after HPLC-based purification and lyophilization as colorless solid (7%). RP-HPLC (10 to 90% B in 15 min): t_{R}=11.3 min. K′=6.06. ESI-MS (positive): calculated monoisotopic mass (C₁₁₈H₁₈₃FN₂₈O₂₉Si): 2505.0. found: m/z=836.3 [M+3H]³⁺, 627.5 [M+4H]⁴⁺.

### DOTAGA-D-Dap[(5-SiFA)Ip]-EDA-DOTA-[α-Me-Trp⁸]MJ9 (4)

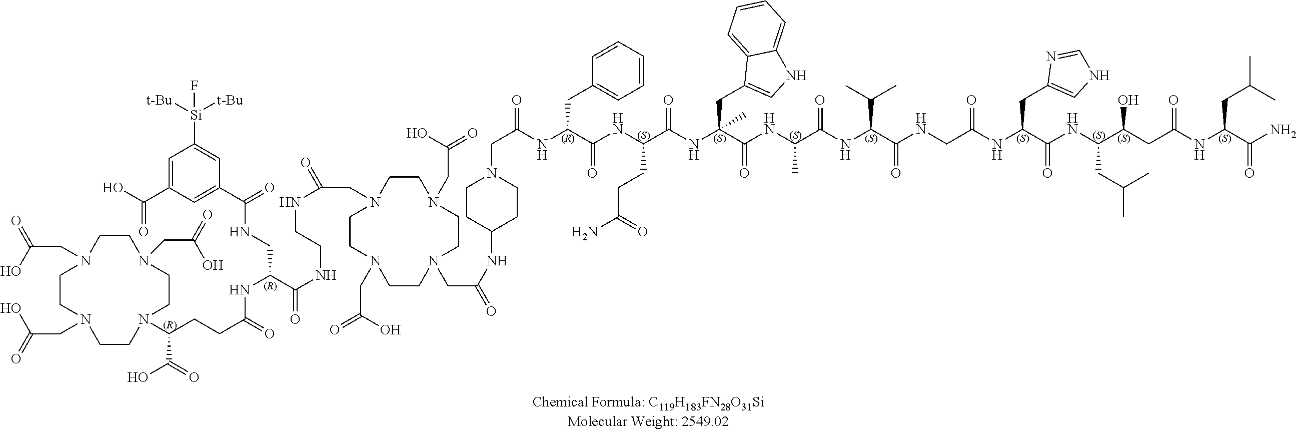

AIIO-(5-SiFA)Ip-OH was coupled to the unprotected Dap-sidechain. Subsequently, the Fmoc protecting group was removed (GP3) and DOTAGA(tBu)₄ was coupled for a prolonged time of 6 h at rt (modified GP2a). The peptide was Allyl-deprotected (GP6), cleaved off the resin under concomitant deprotection (GP5a) and isolated after HPLC-based purification and lyophilization as colorless solid (5%). RP-HPLC (10 to 90% B in 15 min): t_{R}=10.6 min. K′=5.63. ESI-MS (positive): calculated monoisotopic mass (C₁₁₉H₁₈₃FN₂₈O₃₁Si): 2549.0. found: m/z=851.0 [M+3H]³⁺, 638.5 [M+4H]⁴⁺.

### Synthesis of CXCR4 Ligands

CPCR4 (cyclo[Gly-L-2NaI-L-Arg-N(Me)-D-Orn-D-Tyr]) (1)

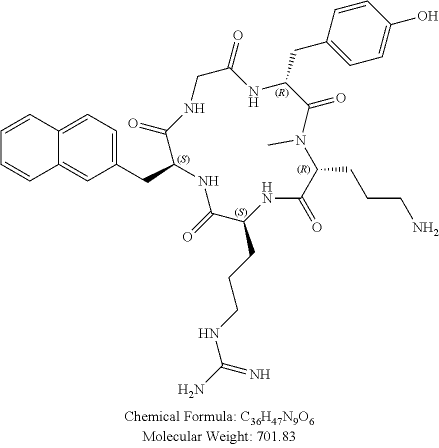

1 was synthesized using solid phase peptide synthesis following the general procedures (GPs). In short, Fmoc-Gly-OH was loaded on the resin (GP1a) und Fmoc-deprotected (GP3). Consecutively, Fmoc-L-2NaI-OH, Fmoc-L-Arg(Pbf)-OH and Fmoc-D-Orn(Boc)-OH were coupled following GP2a for the coupling steps and GP3 for Fmoc-deprotection, alternately. p-Nosyl protection followed by N-Methylation and p-Nosyl deprotection were performed following GP7-9. Fmoc-D-Tyr(tBu)-OH was coupled and Fmoc-deprotected following GP2a and GP3. Cleavage of the resin under retention of protecting groups was performed in analogy to GP5b. The peptide was cyclized (GP12) and side chain deprotected (GP13) in a final step. 1 was obtained as colourless solid (68.3%) and was used without further purification. RP-HPLC (10 to 90% B in 15 min): t_{R}=6.2 min, K′=1.48. ESI-MS (positive): calculated monoisotopic mass (C₃₆H₄₇N₉O₆): 701.36. found: m/z=702.6 [M+H]⁺.

### H₂N-Amba-CPCR4 (2)

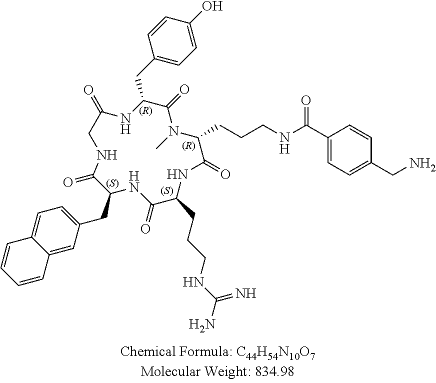

Fmoc-Amba-OH was coupled to 1 following GP10. After reaction completion, the solvent was removed under reduced pressure and Fmoc-deprotected following GP11. 2 was obtained after HPFC-purification and lyophilization as colourless solid (38%). RP-HPLC (10 to 90% B in 15 min): t_{R}=6.3 min, K′=1.86. ESI-MS (positive): calculated monoisotopic mass (C₄₄H₅₄N₁₀O₇): 834.42. found: m/z=835.4 [M+H]⁺.

### (4-SiFA)Bz-EDA-DOTA(tBu)₂-OH (3)

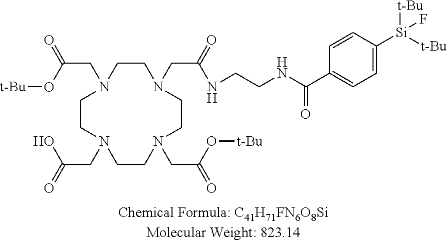 [SMILES: CC(C)(C)OC(=O)CN1CCN(CC(=O)O)CCN(CC(=O)OC(C)(C)C)CCN(CC(=O)NCCNC(=O)c2ccc([Si](F)(C(C)(C)C)C(C)(C)C)cc2)CC1]

DOTA-di(tBu)-OH was coupled to the resin following GP1c. Fmoc-EDA-NH₂ was coupled in analogy to GP2b and Fmoc-deprotected following GP3. (4-SiFA)Bz-OH was coupled following GP2a and cleaved off the resin under retention of protecting groups (GP5b). 3 was obtained after HPFC-purification and lyophilization as colourless solid (36%). RP-HPLC (10 to 90% B in 15 min): t_{R}=11.8 min, K′=4.13. ESI-MS (positive): calculated monoisotopic mass (C₄₁H₇₁FN₆O₈Si): 822.51. found: m/z=823.7 [M+H]⁺.

### AIIO-(5-SiFA)Ip-EDA-DOTA(tBu)₂-OH (4)

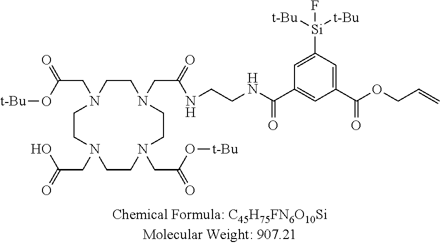 [SMILES: C=CCOC(=O)c1cc(C(=O)NCCNC(=O)CN2CCN(CC(=O)OC(C)(C)C)CCN(CC(=O)O)CCN(CC(=O)OC(C)(C)C)CC2)cc([Si](F)(C(C)(C)C)C(C)(C)C)c1]

DOTA-di(tBu)-OH was coupled to the resin following GP1c. Fmoc-EDA-NH₂ was coupled in analogy to GP2b and Fmoc-deprotected following GP3. AIIO-(5-SiFA)Ip-OH was coupled following GP2a and cleaved off the resin under retention of protecting groups (GP5b). After HPFC-purification and lyophilization 4 was obtained as a colourless solid (23%). RP-HPLC (10 to 90% B in 15 min): t_{R}=12.5 min, K′=4.00. ESI-MS (positive): calculated monoisotopic mass (C₄₅H₇₅FN₆O₁₀Si): 906.53. found: m/z=907.6 [M+H]⁺.

### (4-SiFA)Bz-EDA-DOTA-Amba-CPCR4 (5)

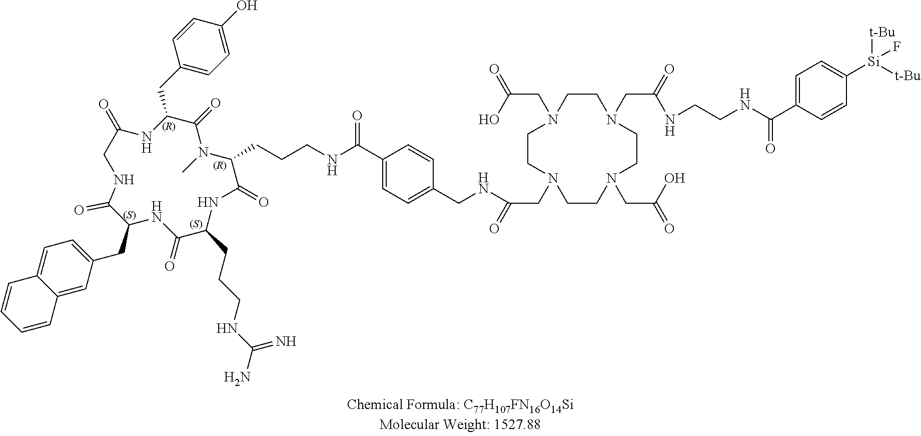

2 was coupled to 3 following GP10 and side chain deprotected following GP13. 5 was obtained after purification via RP-HPLC and lyophilisation as colourless solid (40%). RP-HPLC (10 to 90% B in 15 min): t_{R}=9.80 min, k′=3.08. ESI-MS (positive): calculated monoisotopic mass (C₇₇H₁₀₇FN₁₆O₁₄Si): 1526.79. found: m/z=1528.7 [M+H]⁺, 764.8 [M+2H]²⁺.

### (5-SiFA)Ip-EDA-DOTA-Amba-CPCR4 (6)

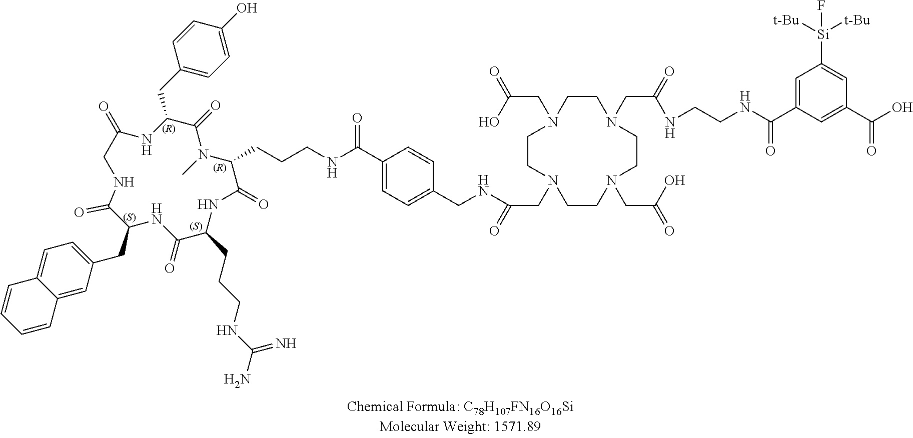

2 was coupled to 4 following GP10 and subsequently Allyl- and side chain-deprotected following GP14 and GP13, respectively. 6 was obtained after purification via RP-HPLC and lyophilisation as colourless solid (19%). RP-HPLC (10 to 90% B in 15 min): t_{R}=9.00 min, K′=3.09. ESI-MS (positive): calculated monoisotopic mass (C₇₈H₁₀₇FN₁₆O₁₆Si): 1570.78. found: m/z=1572.2 [M+H]⁺, 786.9 [M+2H]²⁺.

### Synthesis of SSTR Ligands

Fmoc-D-Phe-cyclo[L-Cys-L-Tyr(tBu)-D-Trp(Boc)-L-Lys(Boc)-L-Thr(tBu)-L-Cys]-L-Thr(tBu)-2-CTC resin
(Fmoc-TATE(PG)-2-CTC resin) (1)

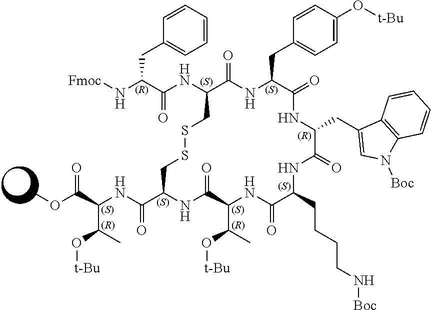

The synthesis of all herein described SSTR ligands started with the same resin-bound precursor H-TATE(PG)-2-CTC resin. In a first step, 2-CTC resin was loaded with Fmoc-L-Thr(tBu)-OH (GP1a). Subsequently, the peptide scaffold was built by alternately coupling the corresponding Fmoc-protected amino acids (GP2a), namely Fmoc-L-Cys(Acm)-OH, Fmoc-L-Thr(tBu)-OH, Fmoc-L-Lys(Boc)-OH, Fmoc-D-Trp(Boc)-OH, Fmoc-L-Tyr(tBu)-OH, Fmoc-L-Cys(Acm)-OH and Fmoc-D-Phe-OH, and their consecutive Fmoc-deprotection (GP3), respectively. The so far completely protected, linear peptide sequence was Acm-deprotected and cyclized (GP15). To confirm the identity of the precursor, a small sample of the resin was treated according to GP5a. RP-HPLC (10 to 90% B in 15 min): t_{R}=10.8 min. K′=5.07. ESI-MS (positive): calculated monoisotopic mass for Fmoc-D-Phe-cyclo[L-Cys-L-Tyr-D-Trp-L-Lys-L-Thr-L-Cys]-L-Thr-OH (C₆₄H₇₄N₁₀O₁₄S₂): 1270.5. found: m/z=1273.4 [M+H]⁺.

### (4-SiFA)Bz-EDA-DOTATATE (2)

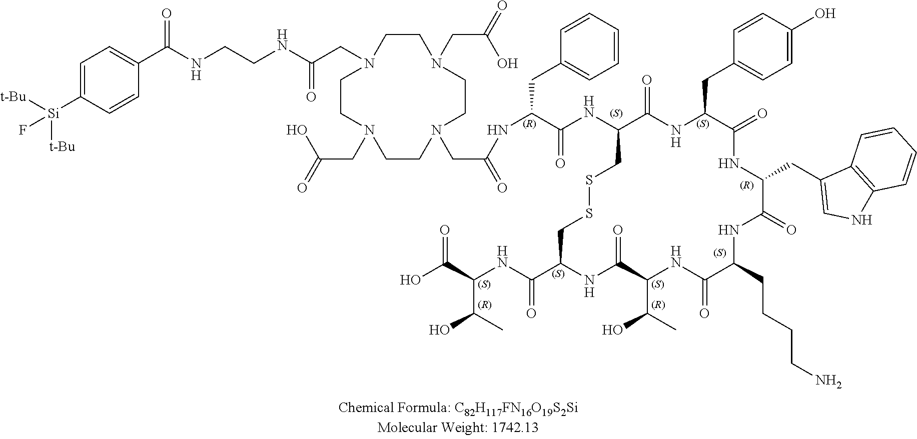

The Fmoc-TATE(PG)-loaded resin (1) was treated according to GP3 for Fmoc-deprotection. DOTA(tBu)₂ was coupled to the resin following GP2a. Fmoc-EDA-NH₂ was coupled in analogy to GP2b and Fmoc-deprotection was done following GP3. (4-SiFA)Bz-OH was coupled following GP2a and the peptide was cleaved off the resin under concomitant cleavage of acid-labile protecting groups (GP5a). The product was obtained after RP-HPLC-purification and lyophilization as a colourless solid (5%). RP-HPLC (10 to 90% B in 15 min): t_{R}=9.8 min, K′=3.00. ESI-MS (positive): calculated monoisotopic mass (C₈₂H₁₁₇FN₁₆O₁₉S₂Si): 1740.8. found: m/z=1741.9 [M+H]⁺, 871.2 [M+2H]²⁺, 581.2 [M+3H]³⁺.

### (4-SiFA)Bz-D-Dap(DOTATATE) (3)

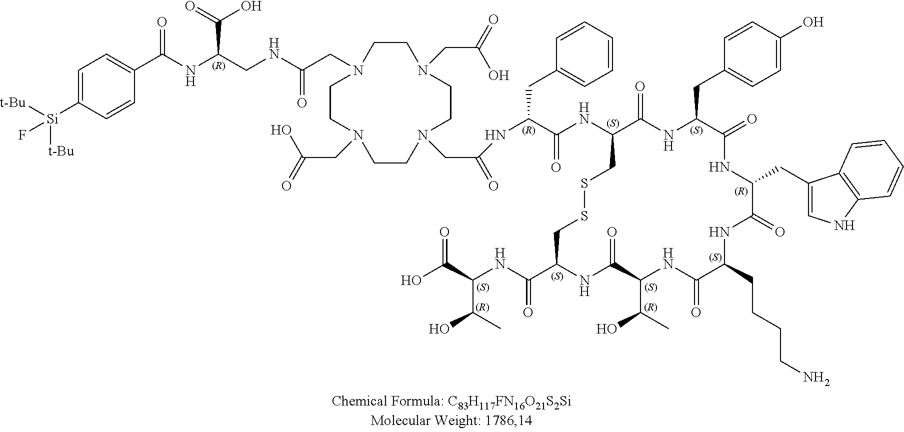

The Fmoc-TATE(PG)-loaded resin (1) was treated according to GP3 for Fmoc-deprotection. DOTA(tBu)₂ was coupled to the resin following GP2a. Fmoc-D-Dap-OtBu-HCl was coupled in analogy to GP2b and Fmoc-deprotection was done following GP3. (4-SiFA)Bz-OH was coupled following GP2a and the peptide was cleaved off the resin under concomitant cleavage of acid-labile protecting groups (GP5a). The product was obtained after RP-HPLC-purification and lyophilization as a colourless solid (8%). RP-HPLC (10 to 90% B in 15 min): t_{R}=9.7 min, K′=2.96. ESI-MS (positive): calculated monoisotopic mass (C₈₃H₁₁₇FN₁₆O₂₁S₂Si): 1784.8. found: m/z=1786.8 [M+H]⁺, 892.9 [M+2H]2+.

### (5-SiFA)Ip-EDA-DOTATATE (4)

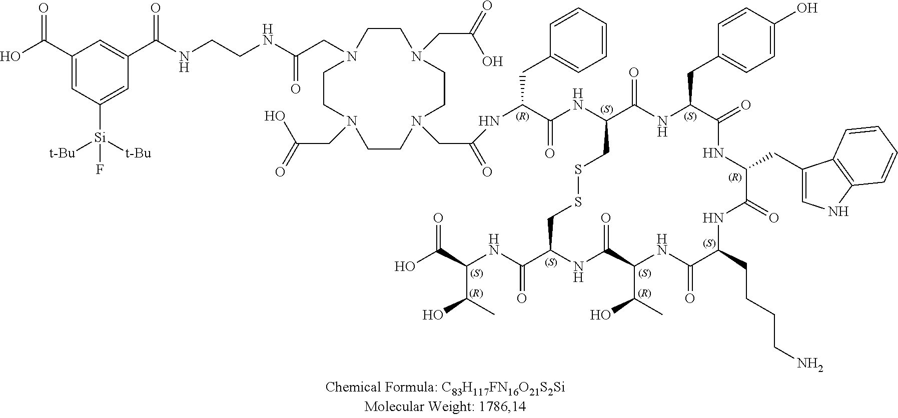

The Fmoc-TATE(PG)-loaded resin (1) was treated according to GP3 for Fmoc-deprotection. DOTA(tBu)₂ was coupled to the resin following GP2a. Fmoc-EDA-NH₂ was coupled in analogy to GP2b and Fmoc-deprotection was done following GP3. (5-SiFA)Ip-OH was coupled following GP2a. The peptide was allyl-deprotected (GP6) and cleaved off the resin under concomitant cleavage of acid-labile protecting groups (GP5a). The product was obtained after RP-HPLC-purification and lyophilization as a colourless solid (4%). RP-HPLC (10 to 90% B in 15 min): t_{R}=9.1 min, K′=2.71. ESI-MS (positive): calculated monoisotopic mass (C₈₃H₁₁₇FN₁₆O₂₁S₂Si): 1784.8. found: m/z=1787.4 [M+H]⁺, 894.1 [M+2H]²⁺.

### (5-SiFA)Ip-D-Dap(DOTATATE) (5)

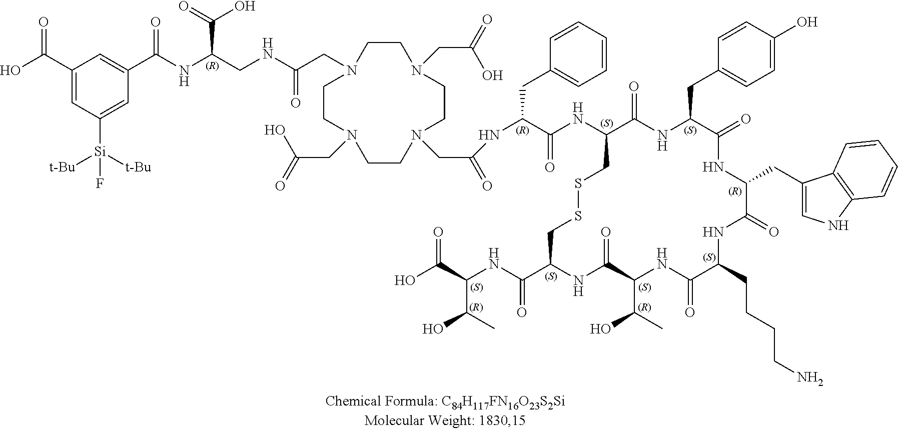

The Fmoc-TATE(PG)-loaded resin (1) was treated according to GP3 for Fmoc-deprotection DOTA(tBu)₂ was coupled to the resin following GP2a. Fmoc-D-Dap-OtBu-HCl was coupled in analogy to GP2b and Fmoc-deprotection was done following GP3. (5-SiFA)Ip-OH was coupled following GP2a. The peptide was allyl-deprotected (GP6) and cleaved off the resin under concomitant cleavage of acid-labile protecting groups (GP5a). The product was obtained after RP-HPLC-purification and lyophilization as a colourless solid (9%). RP-HPLC (10 to 90% B in 15 min): t_{R}=9.0 min, K′=2.67. ESI-MS (positive): calculated monoisotopic mass (C₈₄H₁₁₇FN₁₆O₂₃S₂Si): 1828.8. found: m/z=1832.0 [M+H]⁺, 915.9 [M+2H]²⁺, 611.0 [M+3H]³⁺.

### Synthesis of CCK-2R Ligands

### Fmoc-D-Dap-D-Glu(tBu)-L-Ala-L-Tyr(tBu)-Gly-L-Trp(Boc)-L-Nle-L-Asp(tBU)-L-1-NaI—NH₂ (1)

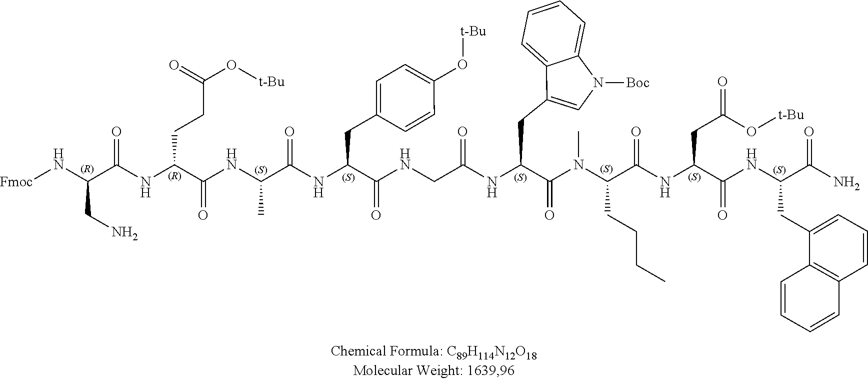

1 was synthesized using solid phase peptide synthesis following the general procedures (GPs). In short, Fmoc-L-1-NaI—OH was coupled to a preloaded H-Rink Amide resin (GP1b), followed by an Fmoc-deprotection (GP3). Afterwards, Fmoc-L-Asp(tBu)-OH and Fmoc-L-Nle-OH were coupled (GP2a) accordingly and the Fmoc-protecting group was cleaved (GP3) after each coupling step. The free N-terminus of Nle was p-Nosyl protected (GP7), N-methylated (GP9) and p-Nosyl deprotected (GP8). Then, Fmoc-L-Trp(Boc)-OH, Fmoc-Gly-OH, Fmoc-L-Tyr(tBu)-OH, Fmoc-L-Ala-OH and Fmoc-D-glu(tBu)-OH were coupled according to GP2a with respective subsequent Fmoc-deprotections (GP3. Fmoc-D-dap(Dde)-OH was coupled to the free amine using 2,4,6-trimethylpyridine as base (GP2a), followed by Dde-deprotection und Fmoc-preservation (GP4b). The resin-bound peptide was used for synthesis without further purification and analytics.

### DOTA-rhCCK-52 (DOTA-D-Dap[(4-SiFA)Bz]-D-Glu-L-Ala-L-Tyr-Gly-L-Trp-L-Nle-L-Asp-L-1-NaI) (2)

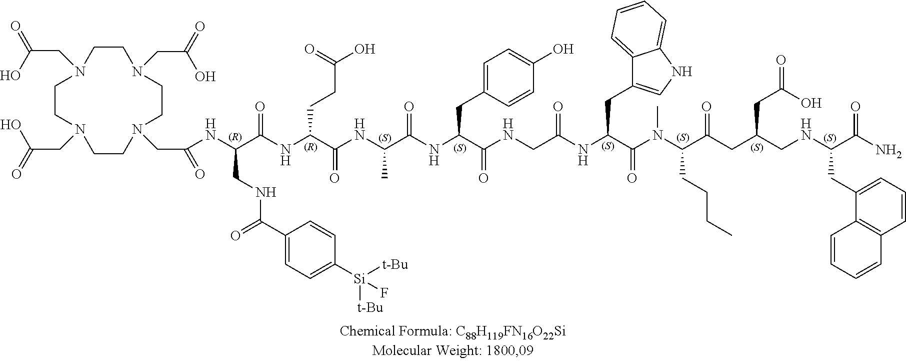

(4-SiFA)Bz-OH was coupled to 1 (GP2a), the Fmoc-protecting group was cleaved (GP3) and DOTA(tBu)₃ was coupled (GP2a). Afterwards the peptide was cleaved from the resin under concomitant deprotection (GP5a) and isolated after RP-HPLC-based purification and lyophilization as colorless solid (6%). RP-HPLC (10 to 70% B in 15 min,): t_{R}=14.5 min, K′=6.25. ESI-MS (positive): calculated monoisotopic mass (C₈₈H₁₁₉FN₁₆O₂₂Si): 1800.1. found: m/z=1800.3 [M+H]⁺, 900.3 [M+2H]²⁺.

### DOTA-rhCCK-53 (DOTA-D-Dap[(3-SiFA)Bz]-D-Glu-L-Ala-L-Tyr-Gly-L-Trp-L-Nle-L-Asp-L-1-NaI) (3)

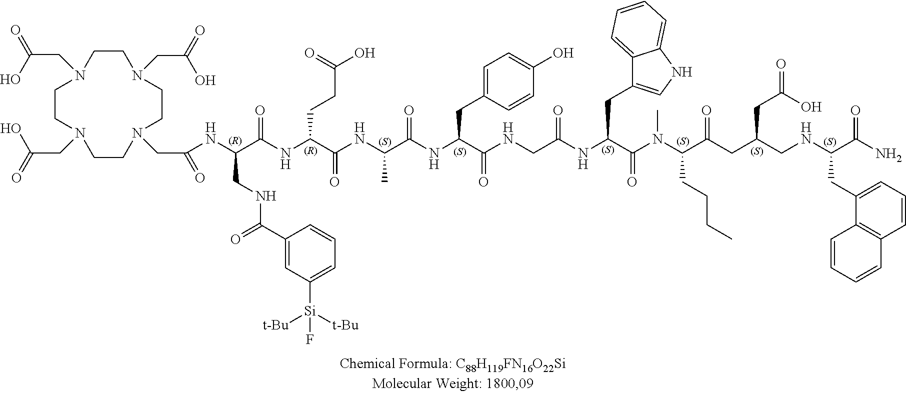

(3-SiFA)Bz-OH was coupled to 1 (GP2a), the Fmoc-protecting group was cleaved (GP3) and DOTA(tBu)₃ was coupled (GP2a). Afterwards the peptide was cleaved from the resin under concomitant deprotection (GP5a) and isolated after RP-HPLC-based purification and lyophilization as colorless solid (9%). RP-HPLC (10 to 70% B in 15 min): t_{R}=14.3 min, K′=6.15. ESI-MS (positive): calculated monoisotopic mass (C₈₈H₁₁₉FN₁₆O₂₂Si): 1800.1. found: m/z=1800.5 [M+H]⁺, 900.3 [M+2H]²⁺.

### DOTA-rhCCK-54 (DOTA-D-Dap[(5-SiFA)Ip]-D-glu-L-Ala-L-Tyr-Gly-L-Trp-L-Nle-L-Asp-L-1-NaI) (4)

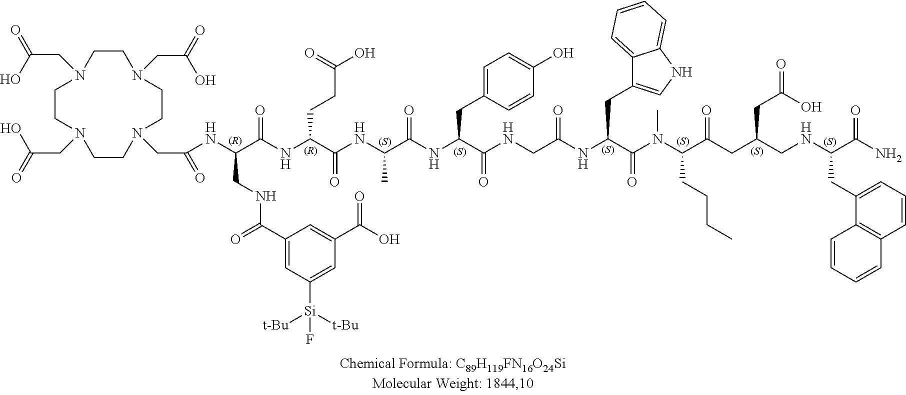

AIIO-(5-SiFA)Ip-OH was conjugated to 1 (GP2a), the Fmoc-protecting group was cleaved (GP3), DOTA(tBu)₃was coupled (GP2a) and the Allyl-protection group was cleaved off (GP6). Afterwards the peptide was cleaved from the resin under concomitant deprotection (GP5a) and isolated after RP-HPLC-based purification and lyophilization as colorless solid (5%). RP-HPLC (10 to 70% B in 15 min): t_{R}=13.6 min, K′=5.80. ESI-MS (positive): calculated monoisotopic mass (C₈₈H₁₁₉FN₁₆O₂₂Si): 1800.1. found: m/z=1845.1 [M+H]⁺, 923.0 [M+2H]²⁺.

### Synthesis of PSMA Ligands

(S)-5-(tert-Butoxy)-4-(3-((S)-1,5-di-tert-butoxy-1,5-dioxopentan-2-yl)ureido)-5-oxopentanoic acid ((tBuO)EuE(OtBu)₂) (prepared according to Wurzer et al. (5))

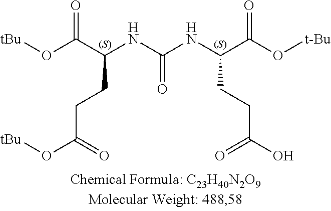

Di-tert-butyl (1H-imidazole-1-carbonyl)-L-glutamate (i)

A solution of 20 mL DCM containing 2.0 g (7.71 mmol, 1.0 eq.) di-tert-butyl-L-glutamate-HCl was cooled on ice for 30 min and afterwards treated with 2.69 mL TEA (19.28 mmol, 2.5 eq.) and 3.3 mg (0.3 mmol, 0.04 eq.) DMAP. After additional stirring for 5 min, 1.38 g (8.84 mmol, 1.1 eq.) of 1,1′-carbonyldiimidazole (CDI) dissolved in DCM were slowly added over a period of 30 min. The reaction mixture was further stirred overnight and enabled to warm to rt. The reaction was stopped using 8 mL saturated NaHCO₃with concomitant washing steps of water (2×30 mL) and brine (2×30 mL) and dried over Na₂SO₄. The remaining solvent was removed in vacuo and the crude product (S)-di-tert-butyl 2-(1H-imidazole-1-carboxamido) pentanedioate (i) was used without further purification.

5-Benzyl 1-(tert-butyl) (((S)-1,5-di-tert-butoxy-1,5-dioxopentan-2-yl)carbamoyl)-L-glutamate (ii)

In 20 mL 1,2-dichloroethane (DCE) 2.72 g (7.71 mmol, 1.0 eq.) of the crude product (S)-di-tert-butyl-2-(1H-imidazole-1-carboxamido)pentanedioate (i) were dissolved and cooled on ice for 30 min. To this solution were added 2.15 mL (15.42 mmol, 2.0 eq.) TEA and 2.54 g (7.71 mmol, 1.0 eq.) H-L-Glu(OBzl)-OtBu·HCl and the solution was stirred overnight at 40° C. The remaining solvent was evaporated and the crude product purified using silica gel flash-chromatography with an eluent mixture containing ethyl acetate/hexane/TEA (v/v/v=500:500:0.8). After removal of the solvent, 5-benzyl-1-(tert-butyl)-(((S)-1,5-di-tert-butoxy-1,5-dioxopentan-2-yl)carbamoyl)-L-glutamate (ii) was obtained as a colorless oil.

(tBuO)EuE(OtBu)₂ (iii)

To synthesize (tBuO)EuE(OtBu)₂, 3.17 g (5.47 mmol, 1.0 eq.) of 5-benzyl-1-(tert-butyl)-(((S)-1,5-di-tert-butoxy-1,5-dioxopentan-2-yl)carbamoyl)-L-glutamate (ii) were dissolved in 75 mL EtOH and 0.34 g (0.57 mmol, 0.1 eq.) palladium on activated charcoal (10%) were given to this solution. The flask containing the reaction mixture was initially purged with H₂ and the solution was stirred over night at rt under light H₂-pressure (balloon). The crude product was filtered through celite and the solvent evaporated in vacuo. The product (iii) was obtained as a hygroscopic solid (84%). RP-HPLC (10 to 90% B in 15 min): t_{R}=11.3 min. ESI-MS (positive): calculated monoisotopic mass (C₂₃H₄₀N₂O₉): 488.3. found: m/z=489.4 [M+H]⁺, 516.4 [M+Na]⁺.

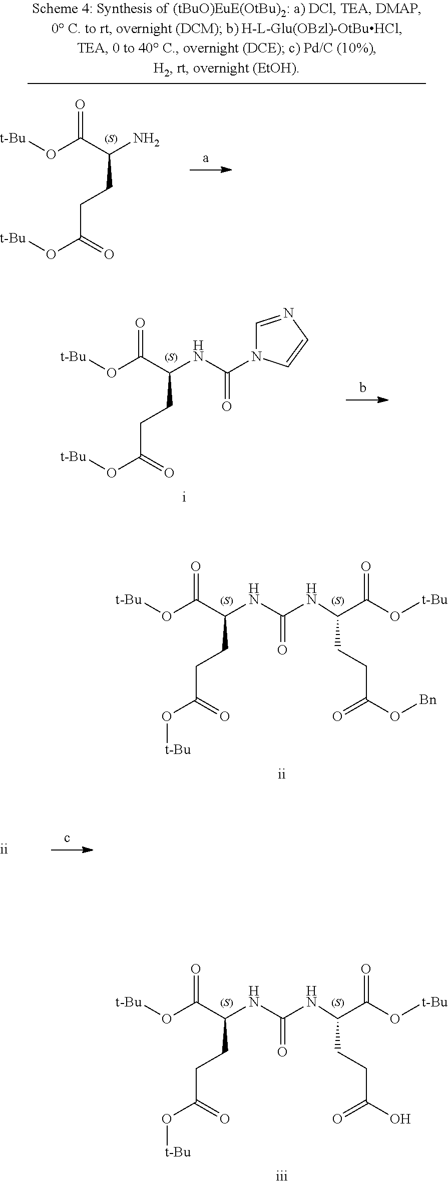

(tBuO)KuE(OtBu)₂ (prepared according to Weineisen et al. (6))

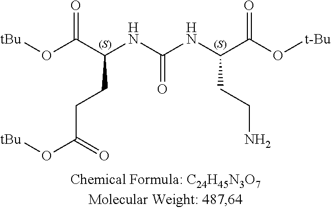

6-(Benzyloxy)carbonyl-1-(tert-butyl) (((S)-1,5-di-tert-butoxy-1,5-dioxopentan-2-yl)carbamoyl)-L-lysinate (i)

In 20 mL 1,2-dichloroethane (DCE) 2.72 g (7.71 mmol, 1.0 eq.) of the crude product (S)-di-tert-butyl-2-(1H-imidazole-1-carboxamido)pentanedioate (see synthesis of (tBuO)EuE(OtBu)₂) were dissolved and cooled on ice for 30 min. To this solution were added 2.15 mL (15.42 mmol, 2.0 eq.) TEA and 2.87 g (7.71 mmol, 1.0 eq.) H-L-Lys(Z)-OtBu-HCl and the solution was stirred overnight at 40° C. The remaining solvent was evaporated and the crude product purified using silica gel flash-chromatography with an eluent mixture containing ethyl acetate/hexane/TEA (v/v/v=500:500:0.8). After removal of the solvent, (i) was obtained as a colorless oil.

(tBuO)KuE(OtBu)₂ (ii)

To synthesize (tBuO)KuE(OtBu)₂, 3.40 g (5.47 mmol, 1.0 eq.) of (i) were dissolved in 75 mL EtOH and 0.34 g (0.57 mmol, 0.1 eq.) palladium on activated charcoal (10%) were given to the solution. The flask containing the reaction mixture was initially purged with H₂ and the solution was stirred over night at rt under light H₂-pressure (balloon). The crude product was filtered through celite and the solvent evaporated in vacuo. The product (ii) was obtained as a hygroscopic solid (92%). RP-HPLC (10 to 90% B in 15 min): t_{R}=12.6 min. K′=6.41. ESI-MS (positive): calculated monoisotopic mass (C₂₄H₄₅N₃O₇): 487.6. found: m/z=488.3 [M+H]⁺, 510.3 [M+Na]⁺.

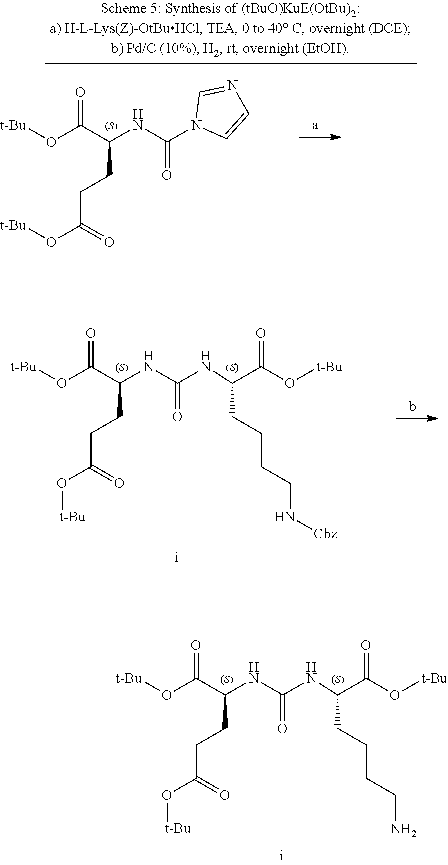

### PSMA-1 (1)

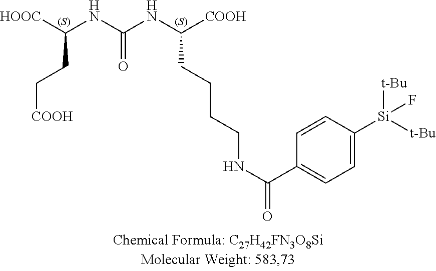

(4-SiFA)Bz-OH (4.2 mg, 14.9 μmol, 1.0 eq.) was pre-activated with HOAt (2.0 mg, 14.9 μmol, 1.0 eq.), TBTU (4.8 mg, 14.9 μmol, 1.0 eq.) and DIPEA (4.5 eq.) in 1 mL DMF for 15 min at rt. For coupling, 500 μL of a solution of (tBuO)KuE(OtBu)₂ (1.0 eq.) in DMF were added to the pre-activated SiFA-building block and stirred for 2 h at rt. Next, the solvent was evaporated under reduced pressure at 60° C. The residue was dissolved in 1 mL of TFA/TIPS/DCM (v/v/v=95:2.5:2.5) and left to react for 1 h at rt. Subsequently, the volatiles were removed under a stream of nitrogen, the dried crude product was purified by RP-HPLC and lyophilized to obtain a colorless solid (3.24 mg, 5.55 mmol, 37%). RP-HPLC (10 to 70% B in 15 min): t_{R}=11.7 min. K′=5.57. ESI-MS (positive): calculated monoisotopic mass (C₂₇H₄₂FN₃O₈Si): 583.3. found: m/z=583.8 [M+H]⁺.

### PSMA-2

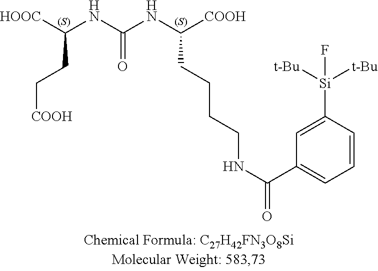

(3-SiFA)Bz-OH (4.2 mg, 14.9 μmol, 1.0 eq.) was pre-activated with HOAt (2.0 mg, 14.9 μmol, 1.0 eq.), TBTU (4.8 mg, 14.9 μmol, 1.0 eq.) and DIPEA (4.5 eq.) in 1 mL DMF for 15 min at rt. For coupling, 500 μL of a solution of (tBuO)KuE(OtBu)₂ (1.0 eq.) in DMF were added to the pre-activated SiFA-building block and stirred for 2 h at rt. Next, the solvent was evaporated under reduced pressure at 60° C. The residue was dissolved in 1 mL of TFA/TIPS/DCM (v/v/v=95:2.5:2.5) and left to react for 1 h at rt. Subsequently, the volatiles were removed under a stream of nitrogen, the dried crude product was purified by RP-HPLC and lyophilized to obtain a colorless solid (7.92 mg, 13.57 μmol, 91%). RP-HPLC (10 to 70% B in 15 min): t_{R}=11.5 min. K′=5.46. ESI-MS (positive): calculated monoisotopic mass (C₂₇H₄₂FN₃O₈Si): 583.3. found: m/z=584.4 [M+H]⁺.

### PSMA-3

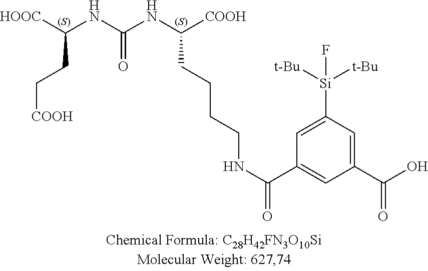

2-CTC resin was loaded with Fmoc-L-Lys(Dde)-OH (GP1). The resin-bound amino acid was Fmoc-deprotected (GP3). On-resin urea-bond formation was conducted with (S)-di-tert-butyl-2-(1H-imidazole-1-carboxamido)pentanedioate according to GP7. The Lys-sidechain was Dde-deprotected (GP4a) and AIIO-(5-SiFA)Ip-OH was coupled (GP2a). The peptide was Allyl-deprotected (GP6) followed by cleaving off the resin with concomitant deprotection of acid-labile protecting groups (GP5a). The cleaved, dried crude product was purified by RP-HPLC and lyophilized to obtain a colorless solid (41%). RP-HPLC (10 to 70% B in 15 min): t_{R}=10.0 min. K′=4.62. ESI-MS (positive): calculated monoisotopic mass (C₂₈H₄₂FN₃O₁₀Si): 627.3. found: m/z=627.8 [M+H]⁺.

rhPSMA-7.3

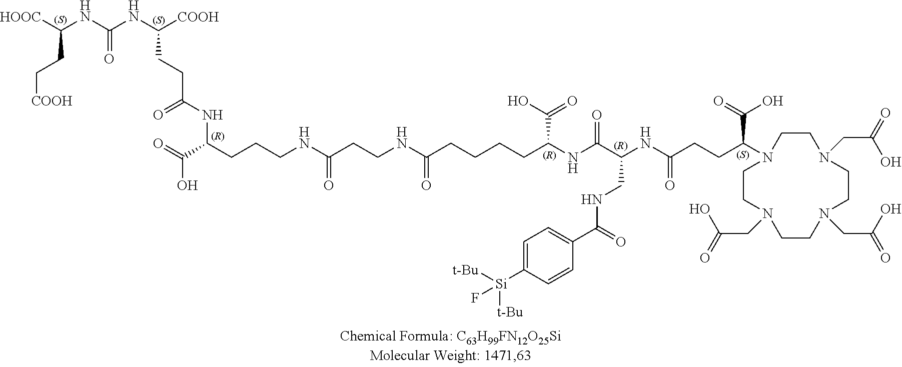

The reference ligandrhPSMA-7.3 was prepared according to the published protocol. (5) RP-HPLC (10 to 70% B in 15 min): t_{R}=10.0 min. K′=4.67. ESI-MS (positive): calculated monoisotopic mass (C₆₃H₉₉FN₁₂O₂₅Si): 1470.7. found: m/z=1471.4 [M+H]⁺, 736.7 [M+2H]²⁺.

### 4.3. Synthesis of Cold Metal Complexes

4.3.1. ^{nat}Ga-Complexes
^{nat}Ga-SSTR Ligands

^{nat}Ga-complexation of SSTR ligands: The purified chelator-containing SSTR ligand (2 mM in DMSO, 1.0 eq.) and ^{nat}Ga(NO₃)₃ (20 mM in Tracepur® H₂O, 3.0 eq.) were diluted with Tracepur® H₂O to a final concentration of 1 mM and heated to 70° C. for 1 h. After cooling, the ^{nat}Ga-chelate formation was confirmed by RP-HPLC/ESI-MS.

^{nat}Ga-(4-SiFA)Bz-EDA-DOTATATE: RP-HPLC (10 to 90% B in 15 min): t_{R}=12.8 min, K′=4.22. ESI-MS (positive): calculated monoisotopic mass (C₈₂H₁₁₅FGaN₁₆O₁₉S₂Si): 1808.1. found: m/z=1809.1 [M+H]⁺, 1206.7 [2M+3H]³⁺, 904.7 [M+2H]²⁺.

^{nat}Ga-(4-SiFA)Bz-D-Dap(DOTATATE): RP-HPLC (10 to 90% B in 15 min): t_{R}=10.1 min, K′=3.12. ESI-MS (positive): calculated monoisotopic mass (C₈₃H₁₁₅FGaN₁₆O₂₁S₂Si): 1852.2. found: m/z=1853.2 [M+H]⁺, 1235.3 [2M+3H]³⁺, 927.0 [M+2H]²⁺.

^{nat}Ga-(5-SiFA)Ip-EDA-DOTATATE: RP-HPLC (10 to 90% B in 15 min): t_{R}=9.6 min, K′=3.00. ESI-MS (positive): calculated monoisotopic mass (C₃₃H₁₁₅FGaN₁₈O₂₁S₂Si): 1851.6. found: m/z=1854.2 [M+H]⁺, 1236.6 [2M+3H]³⁺, 927.3 [M+2H]²⁺.

^{nat}Ga-(5-SiFA)Ip-D-Dap(DOTATATE): RP-HPLC (10 to 90% B in 15 min): t_{R}=9.5 min, K′=2.96. ESI-MS (positive): calculated monoisotopic mass (C₈₄H₁₁₅FGaN₁₆O₂₃S₂Si): 1895.7. found: m/z=1897.6 [M+H]⁺, 949.7 [M+2H]²⁺.

^{nat}Ga-CXCR4 ligands

^{nat}Ga-(4-SiFA)Bz-EDA-DOTA-Amba-CPCR4: RP-HPLC (10 to 60% B in 15 min): t_{R}=14.4 min, K′=5.26. ESI-MS (positive): calculated monoisotopic mass (C₇₇H₁₀₇FGaN₁₆O₁₄Si): 1595.7. found: m/z=798.6 [M+2H]²⁺.

^{nat}Ga-(4-SiFA)Bz-EDA-DOTA-Amba-CPCR4: RP-HPLC (10 to 60% B in 15 min): t_{R}=13.8 min, K′=5.00. ESI-MS (positive): calculated monoisotopic mass (C₇₈H₁₀₅FGaN₁₆O₁₆Si): 1637.7. found: m/z=820.3 [M+2H]²⁺.

4.3.2. ^{nat}Lu-Complexes
^{nat}Lu-GRPR Ligands

^{nat}Lu-complexation of GRPR ligands: The purified chelator-containing GRPR ligand (10⁻³ M in Tracepur® H₂O, 1.0 eq.) and ^{nat}LuCl₃ (20 mM in Tracepur® H₂O, 2.5 eq.) were diluted with Tracepur® H₂O to a final concentration of 10⁻⁴ M and heated to 95° C. for 30 min. After cooling, the ^{nat}Lu-chelate formation was confirmed by RP-HPLC/ESI-MS.

^{nat}Lu-DOTAGA-D-Dap((4-SiFA)Bz)EDA-transDOTA-[α-Me-Trp⁸]MJ9: RP-HPLC (10 to 90% B in 15 min): t_{R}=11.4 min, K′=6.13. ESI-MS (positive): calculated monoisotopic mass (C₁₁₈H₁₈₀FLuN₂₈O₂₉Si): 2677.0. found: m/z=951.0 [M+Lu+3H]³⁺.

^{nat}Lu-DOTAGA-D-Dap((3-SiFA)Bz)EDA-transDOTA-[α-Me-Trp⁸]MJ9: RP-HPLC (10 to 90% B in 15 min): t_{R}=11.2 min, K′=6.00; ESI-MS (positive): calculated monoisotopic mass (C₁₁₈H₁₈₀FLuN₂₈O₂₉Si): 2677.0. found: m/z=951.0 [M+Lu+3H]³⁺.

^{nat}Lu-DOTAGA-D-Dap((5-SiFA)Ip)EDA-transDOTA-[α-Me-Trp⁸]MJ9: RP-HPLC (10 to 90% B in 15 min): t_{R}=10.5 min, K′=5.56; ESI-MS (positive): calculated monoisotopic mass (C₁₁₉H¹⁸⁰FLuN₂₈O₃₁Si): 2721.0. found: m/z=965.5 [M+Lu+3H]³⁺.

^{nat}Lu-CCK-2R Ligands

^{nat}Lu-complexation of CCK-2R ligands: The corresponding ^{nat}Lu-complexes of CCK-2R ligands were prepared from a 2 mM solution of the ligand (1.0 eq.) in DMSO with a 20 mM aqueous solution of ^{nat}LuCl₃ (2.5 eq.), heated to 95° C. for 30 min. After cooling, the ^{nat}Lu-chelate formation was confirmed by RP-HPLC/ESI-MS.

^{nat}Lu-DOTA-rhCCK-52: RP-HPLC (10 to 90% B in 15 min): t_{R}=12.7 min, K′=7.88. ESI-MS (positive): calculated monoisotopic mass (C₈₈H₁₁₆FLuN₁₆O₂₂Si): 1972.0. found: m/z=1972.6 [M+H]⁺, 986.8 [M+2H]²⁺.

^{nat}Lu-DOTA-rhCCK-53: RP-HPLC (10 to 90% B in 15 min): t_{R}=12.5 min, K′=7.74; ESI-MS (positive): calculated monoisotopic mass (C₈₈H₁₁₆FLuN₁₆O₂₂Si): 1972.0. found: m/z=1973.2 [M+H]⁺, 986.9 [M+2H]²⁺.

^{nat}Lu-DOTA-rhCCK-54: RP-HPLC (10 to 90% B in 15 min): t_{R}=14.1 min, K′=8.86; ESI-MS (positive): calculated monoisotopic mass (C₈₉H₁₁₆FLuN₁₆O₂₄Si): 2016.1. found: m/z=1008.8 [M+2H]²⁺.

### 4.4. Radiolabeling

### 4.4.1. ¹⁸F-Labeling

The given two steps for the [¹⁸F]F-labeling procedure were previously optimized in our group by Daniel Di Carlo (unpublished data).

### Drying of Aqueous [¹⁸F]Fluoride Solution

An aqueous [¹⁸F]fluoride solution (in vitro-studies: 100-300 MBq, biodistribution study: up to 1.7 GBq; approximately 0.6-2.0 GBq/mL by the time of use; obtained from the Klinikum Rechts der Isar, Munich, Germany), was passed through an SAX cartridge (Sep-Pak Accell Plus QMA Carbonate Plus Light, 46 mg, 40 μm, Waters), which was preconditioned with 10 mL of ultrapure water. The [¹⁸F]fluoride-loaded cartridge was dried by rinsing with 8 mL anhydrous DMSO followed by 10 mL air. For elution with an extraction efficiency of 78±9%, a solution of 40 mg of ammonium formate in 500 μL anhydrous DMSO was slowly passed through the cartridge followed by 5 mL air.

Labeling of SiFA-Modified Peptides with the Dried [¹⁸F]fluoride Solution

For labeling, 125-500 μL of the eluted [¹⁸F]fluoride solution were added to a proportional amount of precursor solution (7.5-30 nmol, 1 mM in anhydrous DMSO). After 5 min at rt, a sample was taken to determine the radiochemical conversion (RCC) by thin-layer chromatography (TLC). The labeling mixture was diluted with 10 mL PBS* (pH 3, adjusted with 1 M aq. HCl) and passed through an Oasis HLB Plus Light cartridge (30 mg, 30 μm, freshly preconditioned with 10 mL EtOH and 10 mL ultrapure H₂O, Waters). followed by 10 mL PBS* and 10 mL air. The mixture of ¹⁸F-labeled peptide and its chemically identical, cold precursor was eluted with 300 μL EtOH/PBS* (v/v=7:3). The radiochemical purity of the ¹⁸F-labeled compound was determined by radio-RP-HPLC and radio-TLC (silica gel 60 F₂₅₄, mobile phase: MeCN/PBS (v/v=3:2), +10% of NaOAc solution (2 M in H₂O), +1% TFA). The radiochemical yield (RCY) was determined using a dose calibrator and calculated considering decay-correction. *NOTE: In cases of subsequent stability studies, the PBS used for purification was substituted by ultrapure H₂O. Only the PBS used for TLC was not substituted as the TLC analysis after the cartridge-based purification displayed a proportion of less than 1% free [¹⁸F]fluoride for all labeled compounds.

### 4.4.2. ¹²⁵I-Labeling

¹²⁵I-Labeling of the Reference Compound [3-[¹²⁵I]I-Tyr⁶]MJ9 for GRPR-Based Assays

The reference ligand for IC₅₀ studies ([3-[¹²⁵I]I-tyr⁶]MJ9) was prepared according to a previously published procedure. (7) which was slightly modified by our group. Briefly, 0.2 mg of the precursor ([tyr⁶]MJ9) was dissolved in 20 μL Tracepur® H2O and 280 μL TRIS buffer (25 mM TRIS HCl, 0.4 M NaCl, pH=7.9). After addition of the solution to a vial containing 150 μg Iodo-Gen® (1,3,4,6-Tetrachloro-3α,6α-diphenylglycouril, surface-bound, Merck KGaA, Darmstadt, Germany), 5.0 μL (16 MBq) [125I]NaI (74 TBq/mmol, 3.1 GBq/mL, 40 mm NaOH, Hartmann Analytic, Braunschweig, Germany) were added. The reaction solution was incubated for 15 min at rt and purified by RP-HPLC. Immediately after purification, sodium ascorbate (0.1 M in Tracepur® H₂O, 10 vol.-%) was added to prevent radiolysis. RP-HPLC (20 to 35% B in 20 min): t_{R}=18.9 min, K′=10.46 ([3-[¹²⁵I]I-tyr⁶]MJ9).

¹²⁵I-Labeling of the Reference Compound [¹²⁵I]I-TOC for SSTR-Based Assays

The iodination was conducted similar to a previously published procedure. (8) For the ¹²⁵I-labeling of TOC, 50-150 μg of TOC were dissolved in 20 μL of DMSO in a 1.5 mL Eppendorf reaction tube (Protein LowBind) and 280 μL of TRIS buffer (25 mM TRIS-HCl, 0.4 mM NaCl, pH 7.5) were added. The solution was transferred to a reaction tube (1.5 mL, Protein LowBind) coated with Iodogen® (150 μg) and 5.00 μL (10-20 MBq) [¹²⁵I]NaI solution (74 TBq/mmol, 40 mM NaOH, Hartmann Analytic, Braunschweig, Germany) were added. After 15 min at rt, the reaction was stopped by separation from the oxidant (Iodogen®). The crude product [¹²⁵I]I-TOC was purified by analytical RP-HPLC. To prevent radiolysis, 10 vol. % Na-ascorbate solution (100 mM in H₂O) were added to the resulting product solution. RP-HPLC (20 to 50% B in 15 min): t_{R}=8.2 min. K′=3.97 ([¹²⁵I]I-TOC).

¹²⁵l-Labeling of the Reference Compound [¹²⁵I]-FC131 for CXCR4-Based Assays

The iodination was conducted according to a previously published procedure. (9) Prior to the iodination step the reaction vial was coated by dissolving 1.5 mg Iodogen (1,3,4,6-tetrachloro-3R,6R-diphenylglycoluril, Pierce, Rockford, IL) in 1.0 mL dry DCM and apportioning in 10 Eppendorf Caps (100 μL iodogen solution each cap). DCM was evaporated under nitrogen flow and the Iodogen-coated Eppendorf caps were stored under nitrogen at −20° C. until further application. In such a prepared reaction vial approximately 0.5 mg (0.5 μmol) of FC131 were added and dissolved in 20 μL DMSO and 200 μL of Tris buffer (25 mM in H₂O). To the solution, 5 μL (23.1 MBq) [¹²⁵I]NaI (74 TBq/mmol, 3.1 GBq/mL 40 mM NaOH, Hartmann Analytic, Braunschweig, Germany) were added and incubated for 15 min at rt. The crude product was isolated from unlabeled FC131 via analytical RP-HPLC to afford the desired product. RP-HPLC (20 to 40% B in 20 min); t_{R}=18.0 min, K′=9.0 ([¹²⁵I]I-FC131).

[¹²⁵I]I-Labeling of the Reference Compound [¹²⁵I]-BA-KuE for PSMA-Based Assays

reference ligand ([¹²⁵I]I-BA)KuE for in vitro studies was prepared according to a previously published procedure. (6,10) For the iodination, the used peracetic acid solution was freshly prepared by the incubation of 50 μL acetic acid in 130 μL of 30 vol.-% H₂O₂ in H₂O for 2 h at rt. Approximately 0.1 mg of the stannylated precursor Sn(n-Bu)₃-BA-(tBuO)KuE(OtBu)₂ was dissolved in a mixture of 20 μL peracetic acid, 5.0 μL [¹²⁵I]NaI (20±5 MBq, 74 TBq/mmol, 40 mM in NaOH, Hartmann Analytic, Braunschweig, Germany), 20 μL MeCN and 10 μL acetic acid. The reaction solution was incubated for 15 min at rt, diluted in 10 mL H₂O and loaded on a Sep-Pak C18-Plus Short cartridge (360 mg, 55-105 μm, Waters, preconditioned with 10 mL MeOH followed by 10 mL H₂O). After purging the cartridge with 10 mL H₂O followed by 10 mL air, the peptide was eluted with 1.5 mL EtOH/MeCN (v/v=1:1). The eluate was evaporated to dryness under a gentle nitrogen stream at 70° C. and treated with 500 μL TFA for 45 min. After removing TFA in a stream of nitrogen, the crude product was purified by radio-RP-HPLC, yielding ([¹²⁵I]I-BA)KuE (10±2 MBq). HPLC (20 to 40% B in 20 min): t_{R}=13.0 min.

### 4.4.3. ¹⁷⁷Lu-Labeling

For ¹⁷⁷Lu-labeling, a previously published procedure was applied with minor modifications. (11) The labeling precursor (1.0 nmol, 5 μL, 0.2 mM in DMSO) was added to 10 μL of AcOH/NaOAc buffer (1.0 M in H₂O, pH 5.5, 1.0 M). Subsequently, 10 to 110 MBq ¹⁷⁷LuCl₃ (Specific Activity (S_{A})>3000 GBq/mg, 740 MBq/mL, 0.04 M HCl, ITM, Garching, Germany) were added and the mixture was filled up to 100 μL with 0.04 M aq. HCl. The reaction mixture was heated for 20 min at 90° C. and after addition of sodium ascorbate (0.1 M in PBS) the radiochemical purity was determined using radio-HPLC and radio-TLC (0.1 M sodium citrate buffer and 1.0 M NH₄OAc/DMF buffer (1/1; v/v)).

### 4.5. In Vitro Experiments

### 4.5.1. Cell Culture

### Cultivation of GRPR⁺ PC-3 Cells

GRPR⁺ PC-3 cells (Merck KGaA, Darmstadt, Germany) were cultivated in Dulbecco's modified eagle's medium/Ham's F-12 (DMEM/F-12, v/v=1/1, with stable glutamine, Biochrom GmbH, Berlin, Germany) supplemented with fetal bovine serum (10%, FBS Superior, Biochrom GmbH, Berlin, Germany) at 37° C. in a humidified 5% CO₂ atmosphere. A mixture of trypsin and ethylenediaminetetraacetic acid (0.05%, 0.02%) in PBS (Biochrom GmbH, Berlin, Germany) was used to harvest cells. Cells were counted with a Neubauer hemocytometer (Paul Marienfeld, Lauda-Königshofen, Germany).

### Cultivation of Jurkat Cells

hCXCR4 positive Jurkat human T-cell leukemia (Merck Millipore, Darmstadt, Germany) were cultivated in RPMI 1640 medium (2.0 g/L NaHCO₃, w/o L-Glutamine, low endotoxin, Biochrom, Berlin, Germany) containing 10% (v:v) FBS Superior (Biochrom GmBH, Berlin, Germany) and kept at 37° C. in a humidified CO₂ atmosphere (5%). For splitting, Jurkat suspension cells were harvested by centrifugation (1300 rpm, 3 min) and resuspended in the cultivation medium. The individual cell cultures were split around two times a week, depending on the growth rate of the cells. The cells were counted using a Neubauer hemocytometer (Paul Marienfeld, Lauda-Königshofen, Germany). All operations were performed under sterile conditions using an MSC-Advantage safety workbench from Thermo Fisher Scientific Inc.

### Cultivation of CHO/Sst2(a) Cells

Stabely Sst2(a)-transfected Chinese hamster ovary (CHO/Sst2(a)) cells were cultivated in monolayers in CELLSTAR® cell culture flasks acquired from Greiner Bio-One GmbH (Frickenhausen, Germany) at 37° C. in a humidified atmosphere (5% CO₂) using a HERAcell 150i-Incubator from Thermo Fisher Scientific Inc. (Waltham, United States). As nutrient medium was used DMEM/F12 GlutaMax medium, supplemented 10% FBS Superior (Biochrom GmbH, Berlin, Germany). Furthermore, a Dulbecco's PBS solution with 0.05% trypsin and 0.1% EDTA (v/v) was applied, to detach the cells for cell passaging. The detached cells were counted using a Neubauer hemocytometer (Paul Marienfeld, Lauda-Königshofen, Germany). For optimal growth, cells were harvested at about 80% confluence. In addition, all operations under sterile conditions were accomplished using an MSC-Advantage safety workbench from Thermo Fisher Scientific Inc.

### Cultivation of AR42J Cells

CCK-2R expressing rat pancreatic cancer cells AR42J (CLS GmbH, Eppelheim, Germany) were cultivated in monolayers in CELLSTAR® cell culture flasks acquired from Greiner Bio-One GmbH (Frickenhausen, Germany) at 37° C. in a humidified atmosphere (5% CO₂) using a HERAcell 150i-Incubator from Thermo Fisher Scientific Inc. (Waltham, United States). As nutrient medium was used RPMI 1640 medium, supplemented with 5 mM L-Glu, 5 mL non-essential amino acids (100×) and 10% FBS Superior (Biochrom GmbH, Berlin, Germany). Furthermore, a Dulbecco's PBS solution with 0.05% trypsin and 0.1% EDTA (v/v) was applied, to detach the cells for cell passaging. The detached cells were counted using a Neubauer hemocytometer (Paul Marienfeld, Lauda-Königshofen, Germany). For optimal growth, cells were harvested at about 80% confluence. In addition, all operations under sterile conditions were accomplished using an MSC-Advantage safety workbench from Thermo Fisher Scientific Inc.

### Cultivation of LNCaP Cells

PSMA-positive LNCaP cells (ACC 256; DSMZ-German Collection of Microorganisms and Cell Cultures GmbH, Braunschweig, Germany) were cultivated in Dulbecco modified Eagle medium/Nutrition Mixture F-12 with Glutamax (1:1) (DMEM-F12, Biochrom GmbH, Berlin, Germany) supplemented with fetal bovine serum (10%, FBS Superior, Biochrom GmbH, Berlin, Germany) and kept at 37° C. in a humidified CO₂-atmosphere (5%). A mixture of trypsin and EDTA (0.05%, 0.02%) in PBS (Biochrom GmbH, Berlin, Germany) was used to harvest cells. Cells were counted with a Neubauer hemocytometer (Paul Marienfeld, Lauda-Königshofen, Germany).

### 4.5.2. Affinity Determinations (IC₅₀)

### GRPR Affinity Studies

For determination of the GRPR affinity on PC-3 cells (IC₅₀), cells were harvested 24±2 h before the experiment and seeded in 24-well plates (1.5×10⁵ cells in 1 mL/well).

After removal of the culture medium, the cells were washed once with 500 μL of HBSS (Hank's balanced salt solution, Biochrom GmbH, Berlin, Germany, with addition of 1% bovine serum albumin (BSA, v/v)) and left in 200 μL HBSS (1% BSA, v/v) for 9 min at room temperature for equilibration. Next, 25 μL per well of solutions, containing either HBSS (1% BSA, v/v) as control or the respective compound in increasing concentration (10⁻¹⁰-10⁻⁴ M in HBSS (1% BSA, v/v)), were added with subsequent addition of 25 μL of [3-[¹²⁵I]I-D-Tyr⁶]MJ9 (2.0 nM) in HBSS (1% BSA, v/v). All experiments were performed in triplicate for each concentration. After 2 h incubation at rt, the experiment was terminated by removal of the medium and consecutive rinsing with 300 μL of HBSS (1% BSA, v/v). The media of both steps were combined in one fraction and represent the amount of free [3-[¹²⁵I]I-tyr⁶]MJ9. Afterwards, the cells were lysed with 300 μL of 1 M NaOH for at least 15 min and united with the 300 μL NaOH of the following washing step. Quantification of bound and free 3-[¹²⁵I]I-D-Tyr⁶-MJ9 was accomplished in a γ-counter. IC₅₀ determination for each conjugate was repeated twice.

hCXCR4 Affinity Studies

For receptor binding assays Jurkat cells were counted, separated from the cultivation medium and resuspended in HBSS (Hanks' balanced salt solution, +1% BSA). 200 μL of the suspension (400.000 cells) were incubated with 25 μL of the reference radio ligand [¹²⁵I]I-FC131 (400.000 cpm/25 μL) and 25 μL of the competitor at different concentrations (10-4-10-10 M), 25 μL HBSS (1% BSA) for the control experiment, respectively. After 2 hours at 4-9° C. temperature, the incubation was terminated via centrifugation (1300 rpm, 3 min.). The cell pellets were washed with HBSS (w 0.35 g/L NaHCO₃, w Ca²⁺, w Mg²⁺, w/o Phenol red) and centrifuged, twice each. Radioactivity for both supernatant/wash and cell bound fraction was determined by using a gamma counter. The experiment was performed in triplicate with n=3 per concentration. IC₅₀ values were calculated using GraphPad PRISM software (GraphPad Software Inc., La Jolla, United States).

### SSTR Affinity Studies

To determine the SSTR affinity, CHO/Sst2(a) cells (1.0×10⁵) were seeded into 24-well plates 24±2 h prior to testing, using 1 mL of nutrient medium (DMEM/F12 GlutaMax medium, supplemented 10% FBS Superior) for the cell incubation at 37° C. in a humidified atmosphere (5% CO₂).

After removal of the medium, each well was washed with 300 μL HBSS (1% BSA, v/v). For the cellular assay, 200 μL of the assay medium HBSS (1% BSA, v/v), [¹²⁵]I-TOC (1 nM in HBSS (1% BSA), 25 μL, 0.3 μmol) as a radiolabeled reference and 25 μL of the peptide of interest in increasing concentrations (10⁻¹⁰ to 10⁻⁴ M) in triplicate were added to the cells. Thereafter, the assay was incubated for 1 h at rt and the supernatant was collected. The cells were washed with 300 μL ice-cold PBS and the collected fractions were unified. After lysis of the cells with NaOH (300 μL, 1 N) for 20 min, the respective wells were washed with NaOH (300 μL, 1 N) and both fractions were unified. The radioactivity of both, the supernatant and the lysed fractions were quantified using a γ-counter. The obtained data were evaluated via the GraphPad PRISM software (GraphPad Software Inc., La Jolla, United States), which calculates the halfmaximal inhibitory concentration (IC₅₀) of the peptides.

### CCK-2R Affinity Studies

To determine the receptor affinity of the numerous peptides, AR4-2J cells (2.0×10⁵) were seeded into 24-well plates 24±2 h prior to testing, using 1 mL of nutrient medium (RPMI 1640, 5 mM L-Glu, 5 mL non-essential amino acids (100×), 10% FCS) for the cell incubation at 37° C. in a humidified atmosphere (5% CO₂).

After removal of the medium, each well was washed with 500 μL PBS. For the cellular assay, 200 μL of nutrient medium (RPMI 1640, 5 mM L-Glu, 5 mL non-essential amino acids (100×), 10% FCS, 5% BSA), [¹⁷⁷Lu]Lu-DOTA-PP-F11N (25 μL, 0.3 μmol) as a radiolabeled reference and 25 μL of the peptide of interest in increasing concentrations (10⁻¹⁰ to 10⁻⁴ M) in triplicate were added to the cells. Thereafter, the assay was incubated for 3 h at 37° C. and the supernatant was collected. The cells were washed with 300 μL PBS and the collected fractions were unified. After lysis of the cells with NaOH (300 μL, 1 N) for 15 min, the respective wells were washed with NaOH (300 μL, 1 N) and both fractions were unified. The radioactivity of both, the supernatant and the lysed fractions were quantified using a γ-counter. The obtained data were evaluated via the GraphPad PRISM software (GraphPad Software Inc., La Jolla, United States), which calculates the halfmaximal inhibitory concentration (IC₅₀) of the peptides.

### PSMA Affinity Studies

For PSMA affinity (IC₅₀) determinations, the respective ligand was diluted (serial dilution 10⁻⁴ to 10⁻¹⁰M) in Hank's balanced salt solution (HBSS, Biochrom GmbH, Berlin, Germany), supplemented with 1% bovine serum albumin (BSA, Biowest, Nuaillé, France). In the case of metal-complexed ligands, the crude reaction mixture was diluted analogously, without further purification. Cells were harvested 24±2 hours prior to the experiment and seeded in 24-well plates (1.5×10⁵ cells in 1 mL/well). After removal of the culture medium, the cells were carefully washed with 500 μL of HBSS (1% BSA) and left 15 min on ice for equilibration in 200 μL HBSS (1% BSA). Next, 25 μL per well of solutions, containing either HBSS (1% BSA, control) or the respective test ligand in increasing concentration (10⁻¹⁰-10⁻⁴ M in HBSS) were added with subsequent addition of 25 μL of [¹²⁵I]I-BA-KuE (2.0 nM) as radio-labeled competitor in HBSS (1% BSA). For each concentration, the experiment was performed in triplicate. After incubation on ice for 60 min, the experiment was terminated by removal of the medium and consecutive rinsing with 200 μL of HBSS (1% BSA). The media of both steps were combined in one fraction and represent the amount of free radioligand. Afterwards, the cells were lysed with 250 μL of NaOH (1 M in H₂O) for at least 15 min. After a washing step with NaOH (250 μL, 1 M in H₂O), both fractions, representing the amount of bound ligand, were united. Quantification of all collected fractions was accomplished in a γ-counter. IC₅₀ values were calculated using GraphPad PRISM software (GraphPad Software Inc., La Jolla, United States). The whole PSMA-affinity determination was carried out at least three times per ligand.

### 4.5.3. Internalization Studies

### CCK-2R Internalization Studies

For the determination of the internalization kinetics of the various peptides, AR42J cells (3.0×10⁵) were seeded into poly-L-lysine coated 24-well plates adding 1 mL of nutrient medium (RPMI 1640, 5 mM L-Glu, 5 mL non-essential amino acids (100×), 10% FCS). Afterwards, the cells were incubated for 24±2 h at 37° C. in a humidified atmosphere (5% CO₂).

On the day of the experiment, the medium was removed, and each well was washed with incubation medium (RPMI 1640, 5 mM L-Glu, 5 mL non-essential amino acids (100×)) (300 μL). Afterwards 200 μL of nutrient medium (RPMI 1640, 5 mM L-Glu, 5 mL non-essential amino acids (100×), 10% FCS), 25 μL of the [¹⁷⁷Lu]Lu-labeled peptide (0.3 μmol, n=6) and 25 μL of nutrient medium (RPMI 1640, 5 mM L-Glu, 5 mL non-essential amino acids (100×), 10% FCS) for internalization studies (n=3) respective 25 μL of DOTA-PP-F11N (10 μmol) for blockage studies (n=3) were added. Thereafter, the assay was incubated for 6 h at 37° C. in a humidified atmosphere (5% CO₂). After incubation, the cells were put on ice and the supernatant was collected. Then, the cells were washed with an ice-cold incubation medium (RPMI 1640, 5 mM L-Glu, 5 mL non-essential amino acids (100×)) (300 μL) and both fractions were unified. To displace the peptides from the cell membrane, 300 μL of an ice-cold glycine buffer (1 M, pH 2.2) were added and the cells were incubated for 15 min on ice. Afterwards, the supernatant was collected, and the cells were washed with 300 μL of the ice-cold glycine buffer (1 M, pH 2.2). Both fractions were unified. After lysis of the cells with NaOH (300 μL, 1 N) for 15 min, the respective wells were washed with NaOH (300 μL, 1 N) and both fractions were unified. The radioactivity of the supernatant, the acid wash and the lysed fractions were quantified using a γ-counter.

4.5.4. Determination of the Relative Radiochemical Conversion (rRCC)

In a first step, the aqueous [¹⁸F]fluoride solution provided by the Klinikum Rechts der Isar was dried according to the aforementioned procedure (4.4.1 [¹⁸F]F-Labeling). For a competitive labeling reaction, 167 μL of the dried [¹⁸F]fluoride solution (30-50 MBq) in DMSO were added to 10 μL of an equimolar mixture of the test compound and the corresponding (4-SiFA)Bz-reference compound (5 nmol each in DMSO). After 5 min at rt, a sample was taken and instantly analyzed via radio-RP-HPLC. If necessary, the standard solvent gradient (10 to 90% B in 15 min) was modified to achieve baseline-separation ((3-SiFA)Bz-Gly-L-X vs. (4-SiFA)Bz-Gly-L-X with X=Tyr: 45 to 65% B in 15 min; X=Asp: 40 to 60% B in 15 min; X=Lys: 35 to 55% B in 15 min). In these cases, a mixture of the cold precursors with an approximate 3-fold excess of (3-SiFA)Bz-compound was analyzed with the corresponding gradients for unambiguous peak assignment. The relative radiochemical conversion (rRCC) was calculated by peak integration and is expressed as the ratio of test-to-reference-compound [%]. The experiment was repeated (n=3).

4.5.5. Determination of the n-Octanol-Water Partition Coefficient (log D_{7.4})

Approximately 1 MBq of the [¹⁷⁷Lu]Lu-labeled tracer or 0.5 MBq of the [¹⁸F]F-labeled tracer was dissolved in 1 mL of n-octanol/PBS (v/v=1:1) in a reaction vial (1.5 mL). After vigorous mixing of the suspension for 3 min at rt, the vial was centrifuged at 15000 g for 5 min (Biofuge 15, Heraus Sepatech, Osterode, Germany) and 100 μL aliquots of both layers were measured in a γ-counter. The experiment was repeated (n≥6).

### 4.5.6. Determination of Human Serum Albumin (HSA) Binding by High Performance Affinity Chromatography (HPAC)

HSA binding of the PSMA-addressing ligands by HPAC was determined according to a previously published procedure via HPLC. (12) A Chiralpak HSA column (50×3 mm, 5 μm, H13H-2433, Daicel, Tokyo, Japan) was used at a constant flow rate of 0. mL/min at rt. Mobile phase A was a freshly prepared 50 mM aqueous solution of NH₄OAc (pH 6.9) and mobile phase B was isopropanol (HPLC grade, VWR). The applied gradient for all experiments was 100% A (0 to 3 min), followed by 80% A (3 to 40 min). Prior to the experiment, the column was calibrated using nine reference substances with a HSA binding, known from literature, in the range of 13 to 99%. (12,13). All substances were dissolved in a 1:1 mixture (v/v) of isopropanol and a 50 mM aqueous solution of NH₄OAc (pH 6.9) with a final concentration of 0.5 mg/mL. Non-linear regression was established with the OriginPro 2016G software (Northampton, United States).

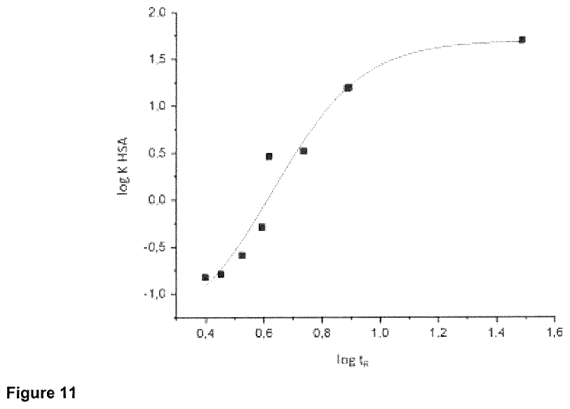

FIG. 11 shows an exemplary sigmoidal plot, showing the correlation between human serum albumin (HSA) binding of selected reference substances and retention time (t_{R}). The values of HSA binding as shown in the following table were obtained from literature (lit. HSA [%]). (12,13)

TABLE 6
Exemplary table with data of reference compounds used for calibration:
Log t_{R}: logarithmic value of experimentally determined retention time
t_{R}; lit. HSA [%]: HSA binding values obtained from literature
(12, 13) Log K HSA: logarithmic value of HSA binding values.

| Reference | t_{R} [min] | Log t_{R} | lit. HSA [%] | Log K HSA |
|---|---|---|---|---|
| p-Benzyl Alcohol | 2.50 | 0.40 | 13.15 | −0.82 |
| Aniline | 2.83 | 0.45 | 14.06 | −0.79 |
| Phenol | 3.35 | 0.52 | 20.69 | −0.59 |
| Benzoic Acid | 3.92 | 0.59 | 34.27 | −0.29 |
| Carabamazepine | 4.15 | 0.62 | 75.00 | 0.46 |
| p-Nitrophenol | 5.45 | 0.74 | 77.65 | 0.52 |
| Estradiol | 7.71 | 0.89 | 94.81 | 1.19 |
| Probenecid | 7.78 | 0.89 | 95.00 | 1.20 |
| Glibenclamide | 30.76 | 1.49 | 99.00 | 1.69 |

4.5.7. Determination of the [¹⁸F]F-Si Bond Stability (Half-Life of ¹⁸F-Defluorination)

### ¹⁸F-Defluorination Under Chemically Simulated In Vivo-Conditions of Blood (pH 7.4, 37° C.)

The test compound was labeled according to the aforementioned procedure (4.4.1 ¹⁸F-Labeling) starting with 200-300 MBq of aqueous [¹⁸F]fluoride solution. The [¹⁸F]F-labeled compound (10 μL per vial, approximately 0.8-1.0 nmol, 2.5-5.0 MBq) was added to 90 μL K₂CO₃ solution (3 mM in ultrapure H₂O, pH 7.4 adjusted with formic acid) per vial. The pH-value of the incubation solution was controlled by a test strip (resolution: 0.5). After incubation for 0, 30, 60, 90 and 120 min at 37° C. in separate reaction vials, samples were analyzed via radio-TLC (silica gel 60 F₂₅₄, mobile phase: MeCN/PBS (v/v=3:2), +10% of NaOAc solution (2 M in H₂O), +1% TFA) differentiating between released [¹⁸F]fluoride, immobilized at the baseline, and labeled test-compound. The experiment was repeated (n=3). The proportions of intact ¹⁸F-labeled peptide at the different time points were used to calculate the half-life of the ¹⁸F-defluorination. For the evaluation, the GraphPad PRISM software (GraphPad Software Inc., La Jolla, United States) was used.

¹⁸F-Defluorination Under ¹⁷⁷Lu-Labeling Conditions for Radiohybrids (pH 5.5, 90° C.)

The test compound was labeled according to the aforementioned procedure (4.4.1 ¹⁸F-Labeling) starting with 200-300 MBq of aqueous [¹⁸F]fluoride solution. The ¹⁸F-labeled compound (10 μL per vial, approximately 0.8-1.0 nmol, 2.5-5.0 MBq) was added to a solution of 80 μL aqueous HCl (40 mM) and 10 μL AcOH/NaOAc buffer (1.0 M in H₂O, pH 5.5, 1.0 M) per vial. The pH-value of the incubation solution was controlled by a test strip (resolution: 0.5). After incubation for 0, 30, 60, 90 and 120 min at 37° C. in separate reaction vials, samples were analyzed via radio-TLC (silica gel 60 F₂₅₄, mobile phase: MeCN/PBS (v/v=3:2), +10% of NaOAc solution (2 M in H₂O), +1% TFA) differentiating between released [¹⁸F]fluoride, immobilized at the baseline, and labeled test-compound. The experiment was repeated (n=3). The proportions of intact ¹⁸F-labeled peptide at the different time points were used to calculate the half-life of the ¹⁸F-defluorination. For the evaluation, the GraphPad PRISM software (GraphPad Software Inc., La Jolla, United States) was used.

¹⁸F- to ^{nat}F Isotopic Exchange in Aqueous Solution (1 mM NaF, pH 6.5, Rt)

The test compound was labeled according to the aforementioned procedure (4.4.1 ¹⁸F-Labeling) starting with 200-300 MBq of aqueous [¹⁸F]fluoride solution. The ¹⁸F-labeled compound (10 μL per vial, approximately 0.8-1.0 nmol, 2.5-5.0 MBq) was added to a solution of 80 μL ultrapure H₂O and 10 μL NaF solution (10 mM in H₂O) per vial. The pH-value of the incubation solution was controlled by a test strip (resolution: 0.5). After incubation for 0, 30, 60, 90 and 120 min at 37° C. in separate reaction vials, samples were analyzed via radio-TLC (silica gel 60 F₂₅₄, mobile phase: MeCN/PBS (v/v=3:2), +10% of NaOAc solution (2 M in H₂O), +1% TFA) differentiating between released [¹⁸F]fluoride, immobilized at the baseline, and labeled test-compound. The experiment was repeated (n=3). The proportions of intact ¹⁸F-labeled peptide at the different time points were used to calculate the half-life of the ¹⁸F-defluorination. For the evaluation, the GraphPad PRISM software (GraphPad Software Inc., La Jolla, United States) was used.

### 4.6. Biodistribution Studies

All animal experiments were conducted in accordance with general animal welfare regulations in Germany (German animal protection act, as amended on 18 May 2018, Art. 141 G v. 29.3.2017 I 626, approval no. 55.2-1-54-2532-71-13) and the institutional guidelines for the care and use of animals. To establish tumor xenografts, LNCaP cells (approximately 1.5×10⁷ cells) were suspended in 200 μL of a 1:1 mixture (v/v) of DMEM F-12 and Matrigel (BD Biosciences, Germany), and inoculated subcutaneously onto the right shoulder of 6-8 weeks old CB17-SCID mice (Charles River, Sulzfeld, Germany). Mice were used for experiments when tumors had grown to a diameter of 2-6 mm (4-20 weeks after inoculation).

Approximately 2-5 MBq (50-120 μmol) of the ¹⁷⁷Lu-labeled ligand or approximately 1-2 MBq (50-120 μmol) of the ¹⁸F-labeled ligand were administered by injection into the tail vein of LNCaP tumor-bearing male CB-17 SCID mice and sacrificed after 1 h or 24 h post injection (p.i.). Selected organs were removed, weighted and measured in a γ-counter. Results are all decay-corrected and given in % injected dose per gram of tissue (% ID/g).

### 5. REFERENCES

1. Wirtz M. Development of biomarkers for molecular imaging and endoradiotherapy of prostate cancer. PhD thesis, Technical University Munich. 2015.
2. Wurzer A, Parzinger M, Konrad M, et al. Preclinical comparison of four [¹⁸F, ^{nat}Ga]rhPSMA-7 isomers: influence of the stereoconfiguration on pharmacokinetics. EJNMMI Research. 2020; 10:149.
3. Niedermoser S, Chin J, Wängler C, et al. In Vivo Evaluation of ¹⁸F-SiFAlin-Modified TATE: A Potential Challenge for ⁶⁸Ga-DOTATATE, the Clinical Gold Standard for Somatostatin Receptor Imaging with PET. J Nucl Med. 2015; 56:1100.
4. Ilhan H, Lindner S, Todica A, et al. Biodistribution and first clinical results of ¹⁸F-SiFAlin-TATE PET: a novel ¹⁸F-labeled somatostatin analog for imaging of neuroendocrine tumors. Eur J Nucl Med Mol Imaging. 2020; 47:870-880.
5. Wurzer A, DiCarlo D, Schmidt A, et al. Radiohybrid ligands: a novel tracer concept exemplified by ¹⁸F- or ⁶⁸Ga-labeled rhPSMA-inhibitors. J Nucl Med. 2019:jnumed.119.234922.
6. Weineisen M, Simecek J, Schottelius M, Schwaiger M, Wester H-J. Synthesis and preclinical evaluation of DOTAGA-conjugated PSMA ligands for functional imaging and endoradiotherapy of prostate cancer. EJNMMI Research. 2014; 4:63.
7. Nakagawa T, Hocart S J, Schumann M, et al. Identification of key amino acids in the gastrin-releasing peptide receptor (GRPR) responsible for high affinity binding of gastrin-releasing peptide (GRP). Biochem Pharmacol. 2005; 69:579-593.
8. Schottelius M, Šimeček J, Hoffmann F, Willibald M, Schwaiger M, Wester H J. Twins in spirit—episode 1: comparative preclinical evaluation of [⁶⁸Ga]DOTATATE and [⁶⁸Ga]HA-DOTATATE. EJNMMI Res. 2015; 5:22.
9. Osl T. Development of cyclic pentapeptide ligands for chemokine receptor targeting, PhD thesis, Technical University Munich. 2017.
10. Vaidyanathan G, Zalutsky M R. Preparation of N-succinimidyl 3-[*I]iodobenzoate: an agent for the indirect radioiodination of proteins. Nat Protoc. 2006; 1:707-713.
11. Sosabowski J K, Mather S J. Conjugation of DOTA-like chelating agents to peptides and radiolabeling with trivalent metallic isotopes. Nat Protoc. 2006; 1:972-976.
12. Valko K, Nunhuck S, Bevan C, Abraham M H, Reynolds D P. Fast gradient HPLC method to determine compounds binding to human serum albumin. Relationships with octanol/water and immobilized artificial membrane lipophilicity. J Pharm Sci. 2003; 92:2236-2248.
13. Yamazaki K, Kanaoka M. Computational prediction of the plasma protein-binding percent of diverse pharmaceutical compounds. J Pharm Sci. 2004; 93:1480-1494.

1. A compound comprising a group selected from a group of formula (Ia), (Ib) and (Ic), or a salt thereof: 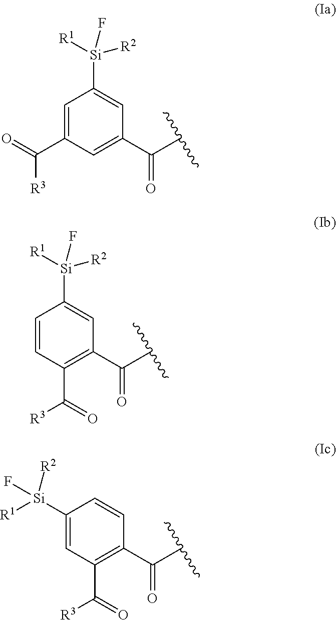
wherein:
R¹ is a linear or branched C3 to C10 alkyl group;
R² is a linear or branched C3 to C10 alkyl group;
R³ is selected from
(i) —OH or —O⁻,
(ii) a sugar moiety or an amino sugar moiety,
(iii) an amino acid moiety or an oligopeptide moiety,
(iv) a PEG moiety;
and from combinations of two or more of (ii), (iii) and (iv);
and the waved line marks a bond which attaches the group to the remainder of the compound.

2. The compound according to claim 1 which is a compound of formula (IIa), (IIb) or (IIc), or a salt of the compound: 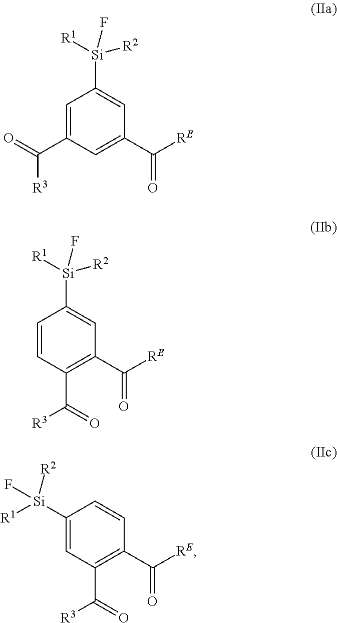
wherein R¹, R², and R³ are defined as in claim 1, and R^{E} is a group which comprises a targeting moiety.

3. The compound or salt according to claim 1, wherein the amino acid moiety as R³ is derived from a hydrophilic amino acid that comprises, in addition to an amino group and a carboxyl group, a further basic or acidic functional group, or wherein the oligopeptide moiety as R³ is derived from an oligopeptide with at least one hydrophilic amino acid that comprises, in addition to an amino group and a carboxyl group, a further basic or acidic functional group.

4. The compound or salt according to claim 3, wherein the hydrophilic amino acid is selected from lysine and glutamic acid.

5. The compound or salt according to claim 1, wherein the oligopeptide moiety as R³ is a linear or branched moiety comprising 2 to 10 amino acid moieties.

6. The compound or salt according to claim 1, wherein the PEG moiety as R³ is a moiety of the formula
—NH—(CH₂—CH₂—O)_{X}—R^{P1},
wherein the nitrogen atom providing an open bond forms an amide bond —NH—C(O)— with the carbon atom to which R³ is attached, X is an integer of 2 to 10, and R^{P1} is selected from —CH₂—COOH and —CH₂—CH₂—COOH.

7. The compound or salt according to claim 1, wherein the sugar moiety or amino sugar moiety as R³ is a residue derived from 6-amino-6-deoxy-D-galactopyranose and corresponding tautomers thereof.

8. The compound or salt according to claim 2, wherein R^{E} comprises a targeting moiety selected from a receptor binding moiety, an enzyme binding substrate or enzyme inhibitor, a peptide, a protein or an antibody fragment or engineered antigen binding construct.

9. The compound or salt according to claim 2, wherein the targeting moiety is a peptidic moiety.

10. The compound or salt according to claim 2, wherein the targeting moiety is a receptor binding moiety which allows the compound of formula (IIa), (IIb) or (IIc) or its salt comprising the targeting moiety to function as a ligand for a receptor selected from a gastrin releasing peptide receptor (GRPR), a C-X-C chemokine receptor type 4 (CXCR4), a somatostatin receptor (SSTR), and a cholecystokinin B receptor (CCK-2R).

11. The compound or salt according to claim 2, wherein R^{E} further comprises a chelating moiety or a chelate moiety formed by the chelating moiety and a chelated radioactive or non-radioactive metal cation.

12. The compound or salt according to claim 11, wherein the chelated metal cation is a radioactive metal cation.

13. The compound or salt according to claim 1, wherein the fluorine attached via a direct covalent bond to the Si atom is [¹⁸F]fluorine.

14-15. (canceled)

16. A method for isotopic exchange of [¹⁹F]fluorine by [¹⁸F]fluorine in a silicon-based fluoride acceptor group, the method comprising exchanging [¹⁹F]fluorine in a group of the formula (Ia), (Ib) or (Ic) as defined in claim 1 with [¹⁸F]fluorine.

17. A method for [¹⁸F]labeling of a targeted radiopharmaceutical, the method comprising using the group of the formula (Ia), (Ib) or (Ic) as defined claim 1 as a silicon-based fluoride acceptor group to thereby label the targeted radiopharmaceutical with [¹⁸F]fluorine.

18. The compound or salt according to claim 5, wherein the oligopeptide moiety as R³ is a linear or branched moiety comprising 2 to 5 amino acid moieties.

19. The compound or salt according to claim 5, wherein the oligopeptide moiety as R³ is a linear or branched moiety comprising 2 or 3 amino acid moieties.
